(19) United States
(12) Patent Application Publication
(10) Pub. No.: US 2018/0312839 A1
(43) Pub. Date: Nov. 1, 2018

(54) METHODS AND COMPOSITIONS FOR INCREASING SMN EXPRESSION

(71) Applicant: TRANSLATE BIO MA, INC., Cambridge, MA (US)

(72) Inventors: Balkrishen Bhat, Carlsbad, CA (US); David Bullough, Encinitas, CA (US)

(73) Assignee: Translate Bio MA, Inc., Lexington, MA (US)

(21) Appl. No.: 15/771,333

(22) PCT Filed: Oct. 26, 2016
(86) PCT No.: PCT/US2016/058828

§ 371 Date: Apr. 26, 2018

Related U.S. Application Data
(60) Provisional application No. 62/369,726, filed on Aug. 1, 2016.
(60) Provisional application No. 62/343,322, filed on May 31, 2016.
(60) Provisional application No. 62/317,385, filed on Apr. 1, 2016.
(60) Provisional application No. 62/246,576, filed on Oct. 26, 2015.

(51) Int. Cl. C12N 15/113; C07K 14/47; C07K 14/475; C12N 15/11

(52) U.S. Cl. CPC C12N 15/113; C07K 14/47; C12N 2830/50; C12N 15/111; C12N 2320/33; C07K 14/475

(57) ABSTRACT

Aspects of the disclosure provide compositions or compounds for activating or enhancing expression of SMN. Further aspects provide compositions and kits, e.g., comprising single stranded oligonucleotides, for activating or enhancing expression of SMN that comprises exon 7. Methods for modulating expression of SMN are also provided.

### CROSS-REFERENCE TO RELATED APPLICATIONS

This application claims the benefit under 35 U.S.C. § 119(e) of U.S. Provisional Application No. U.S. 62/246,576, filed on Oct. 26, 2015, U.S. Provisional Application No. U.S. 62/317,385, filed on Apr. 1, 2016, U.S. Provisional Application No. U.S. 62/343,322, filed on May 31, 2016, and U.S. Provisional Application No. U.S. 62/369,726, filed on Aug. 1, 2016, the contents of each of which are incorporated herein by reference in its entirety.

### FIELD OF THE DISCLOSURE

The disclosure relates to compositions, e.g., oligonucleotide-based compositions, as well as methods of using such compositions, e.g., for treating disease.

### BACKGROUND OF THE DISCLOSURE

Survival of Motor Neuron (SMN) is a protein involved in transcriptional splicing through its involvement in assembly of small nuclear ribonucleoproteins (snRNPs). snRNPs are protein-RNA complexes that bind with pre-mRNA to form a spliceosome, which then splices the pre-mRNA, most often resulting in removal of introns. Three genes encode SMN or a variant of SMN, including SMN1 (survival of motor neuron 1, telomeric), SMN2 (survival of motor neuron 2, centromeric), and SMNP (survival of motor neuron 1, telomeric pseudogene). SMN1 and SMN2 are a result of a gene duplication at 5q13 in humans. A lack of SMN activity results in widespread splicing defects, especially in spinal motor neurons, and degeneration of the spinal cord lower motor neurons.

### SUMMARY OF THE DISCLOSURE

Aspects of the disclosure disclosed herein provide methods and compositions that are useful for upregulating the expression of SMN. In some embodiments, single stranded oligonucleotides are provided that target a PRC2-associated region of a long non-coding RNA that inhibits expression of SMN and thereby causes upregulation of SMN. Also provided herein are methods and related single stranded oligonucleotides that are useful for selectively inducing expression of particular splice variants of SMN. In some embodiments, methods provided herein are useful for controlling the levels in a cell of SMN isoforms encoded by the splice variants. In some cases, the methods are useful for inducing expression of SMN proteins to levels sufficient to treat disease (e.g., SMA).

In some embodiments, single stranded oligonucleotides are provided that target a PRC2-associated region of a long non-coding RNA that inhibits expression of SMN (e.g., human SMN1, human SMN2) and thereby cause upregulation of the gene. For example, according to some aspects of the disclosure methods are provided for increasing expression of full-length SMN protein in a cell for purposes of treating SMA or other motor neuron diseases. Accordingly, aspects of the disclosure provide methods and compositions that are useful for upregulating SMN in cells. In some embodiments, single stranded oligonucleotides are provided that target a PRC2-associated region of a long non-coding RNA that inhibits expression of SMN (e.g., SMN1 and/or SMN2). In some embodiments, these single stranded oligonucleotides activate or enhance expression of SMN by relieving or preventing PRC2-mediated repression of SMN.

In some embodiments, the methods comprise delivering to the cell a first single stranded oligonucleotide complementary with a PRC2-associated region of a long non-coding RNA that inhibits expression of SMN, and a second single stranded oligonucleotide complementary with a splice control sequence of a precursor mRNA of an SMN gene, e.g., a precursor mRNA of SMN, in amounts sufficient to increase expression of a mature mRNA of SMN that comprises (or includes) exon 7 in the cell.

According to some aspects, compositions are provided for increasing expression of SMN protein. In some embodiments, the compositions comprise i) a first oligonucleotide having a nucleotide sequence consisting of 8 to 14 contiguous nucleotides complementary with the nucleotide sequence set forth as: ATCTGTTCCACTATG (SEQ ID NO: 1); and ii) an SMN splice correcting agent (e.g., a splice correcting oligonucleotide) that promotes inclusion of exon 7 of an SMN pre-messenger RNA.

In some embodiments, compounds are provided for increasing expression of SMN protein in a human cell in which the compounds comprise a first oligonucleotide comprising at least 8 contiguous nucleotides complementary with the sequence set forth as: ATCTGTTCCACTATG (SEQ ID NO: 1); and a second oligonucleotide that is complementary with a splice control sequence of SMN pre-messenger RNA and that promotes inclusion of exon 7 of the SMN pre-messenger RNA. In some embodiments, the first and second oligonucleotides are covalently linked. In some embodiments, the first and second oligonucleotides are covalently linked via an oligonucleotide linker. In some embodiments, the oligonucleotide linker comprises a sequence set forth as Wₙ, wherein W is a nucleotide selected from A, T, and U, and n is a integer selected from 2, 3, and 4, representing the number of instances of W. In some embodiments, each instance of W is A. In some embodiments, n is 2 or 3. In some embodiments, the oligonucleotide linker comprises phosphodiester bonds between each instance of W. In some embodiments, the first oligonucleotide has a length in a range of 8 to 14 nucleotides. In some embodiments, the first oligonucleotide has a length in a range of 8 to 10 nucleotides. In some embodiments, the first oligonucleotide comprises at least 8 contiguous nucleotides of the sequence set forth as: AGUGGAACA.

In some embodiments, the second oligonucleotide comprises a region of complementarity complementary with at least 8 contiguous nucleotides of the sequence set forth as: GUAAGUCUGCCAGCAUUAUGAAAG (SEQ ID NO: 2). In certain embodiments, the region of complementarity is complementary with at least 8 contiguous nucleotides of the sequence set forth as: CUGCCAGCAUUAUGAAAG (SEQ ID NO: 3). In certain embodiments, the region of complementarity is complementary with at least 8 contiguous nucleotides of the sequence set forth as: CCAGCAUUAUGAAAG (SEQ ID NO: 4).

According to some aspects of the disclosure, compositions are provided that comprise any of the oligonucleotides disclosed herein, and a carrier. In some embodiments, compositions are provided that comprise any of the oligonucleotides in a buffered solution. In some embodiments, the oligonucleotide is conjugated to the carrier. In some embodiments, the carrier is a peptide. In some embodiments, the carrier is a steroid. According to some aspects of the disclosure, pharmaceutical compositions are provided that comprise any of the oligonucleotides disclosed herein, and a pharmaceutically acceptable carrier.

According to other aspects of the disclosure, kits are provided that comprise a container housing any of the compositions disclosed herein.

According to some aspects of the disclosure, methods of increasing expression of SMN in a cell are provided. In some embodiments, the methods involve delivering any one or more of the single stranded oligonucleotides disclosed herein into the cell. In some embodiments, delivery of the single stranded oligonucleotide into the cell results in expression of SMN that is greater (e.g., at least 50% greater) than expression of SMN in a control cell that does not comprise the single stranded oligonucleotide.

According to some aspects of the disclosure, methods of increasing levels of SMN in a subject are provided. According to some aspects of the disclosure, methods of treating a condition (e.g., ALS, Primary Lateral Sclerosis, Progressive Spinal Muscular Atrophy, Progressive Bulbar Palsy, or Pseudobulbar Palsy) associated with decreased levels of SMN in a subject are provided. In some embodiments, the methods involve administering any one or more of the single stranded oligonucleotides disclosed herein to the subject.

Aspects of the disclosure relate to methods of increasing expression of SMN protein in a cell. In some embodiments, the method comprises delivering to the cell a first single stranded oligonucleotide complementary with at least 8 consecutive nucleotides of a PRC2-associated region of SMN (e.g., SMN2) and a second single stranded oligonucleotide complementary with a splice control sequence of a precursor mRNA of SMN (e.g., SMN2), in amounts sufficient to increase expression of a mature mRNA of SMN that comprises exon 7 in the cell. In some embodiments, the region of complementarity with at least 8 consecutive nucleotides of a PRC2-associated region of SMN (e.g., SMN2) has at least 1, at least 2, at least 3, at least 4, at least 5, at least 6, at least 7, at least 8, or more mismatches with a corresponding region of SMN.

As used herein, the term “splice control sequence” refers to a nucleotide sequence that when present in a precursor mRNA influences splicing of that precursor mRNA in a cell. In some embodiments, a splice control sequence includes one or more binding sites for a molecule that regulates mRNA splicing, such as a hnRNAP protein. In some embodiments, a splice control sequence comprises the sequence CAG or AAAG. In some embodiments, a splice control sequence resides in an exon (e.g., an exon of SMN, such as exon 7 or exon 8). In some embodiments, a splice control sequence traverses an intron-exon junction (e.g., an intron-exon junction of SMN, such as the intron 6/exon 7 junction or the intron 7/exon 8 junction). In some embodiments, a splice control sequence resides in an intron (e.g., an intron of SMN, such as intron 6 or intron 7). In some embodiments, a splice control sequence comprises the sequence: CAGCAUUAUGAAAG (SEQ ID NO: 5) or a portion thereof.

In some embodiments, the first single stranded oligonucleotide and the second single stranded oligonucleotide are delivered to the cell simultaneously. In some embodiments, the cell is in a subject and the step of delivering to the cell comprises administering the first single stranded oligonucleotide and the second single stranded oligonucleotide to the subject as a co-formulation. In some embodiments, the first single stranded oligonucleotide is covalently linked to the second single stranded oligonucleotide through a linker. In some embodiments, the linker comprises an oligonucleotide, a peptide, a low pH-labile bond, or a disulfide bond. In some embodiments, the linker comprises an oligonucleotide, optionally wherein the oligonucleotide comprises 1 to 10 thymidines or uridines. In some embodiments, the linker comprises an oligonucleotide, wherein the oligonucleotide comprises 1 to 10 deoxyadenosines.

In some embodiments, the linker is more susceptible to cleavage in a mammalian extract than the first and second single stranded oligonucleotides. In some embodiments, the first single stranded oligonucleotide is not covalently linked to the second single stranded oligonucleotide. In some embodiments, the first single stranded oligonucleotide and the second single stranded oligonucleotide are delivered to the cell separately.

According to other aspects of the disclosure, kits are provided that comprise a container housing any of the compositions disclosed herein. According to other aspects of the disclosure, kits are provided that comprise a first container housing first single stranded oligonucleotide complementary with at least 8 consecutive nucleotides of a PRC2-associated region of a gene; and a second container housing a second single stranded oligonucleotide complementary to a splice control sequence of a precursor mRNA of the gene. In some embodiments, the splice control sequence resides in an exon of the gene. In some embodiments, the splice control sequence traverses an intron-exon junction of the gene. In some embodiments, the splice control sequence resides in an intron of the gene. In some embodiments, the splice control sequence comprises at least one hnRNAP binding sequence. In some embodiments, hybridization of an oligonucleotide having the sequence of C with the splice control sequence of the precursor mRNA in a cell results in inclusion of a particular exon in a mature mRNA that arises from processing of the precursor mRNA in the cell. In some embodiments, hybridization of an oligonucleotide having the sequence of C with the splice control sequence of the precursor mRNA in a cell results in exclusion of a particular intron or exon in a mature mRNA that arises from processing of the precursor mRNA in the cell. In some embodiments, the gene is SMN. In some embodiments, the splice control sequence resides in intron 6, intron 7, exon 7, exon 8 or at the junction of intron 7 and exon 8 of SMN.

### BRIEF DESCRIPTION OF DRAWINGS

FIG. 1 provides a schematic of SMN1 and SMN2 mRNA processing.

FIG. 2 is a graph of SMN2 mRNA levels in mouse 5025 wild type (WT) cortical neurons following treatment with transcriptional and splice correcting oligonucleotides.

FIGS. 3A to 3B are graphs showing SMN2 mRNA levels (FIG. 3A) and SMN2 protein levels (FIG. 3B) in mouse 5025 wild type (WT) cortical neurons following treatment with splice correctors (e.g., splice correcting oligonucleotides) alone or in combination with transcriptional activator (e.g., transcriptional activating oligonucleotides).

FIGS. 4A-4C shows that the SMN2 locus is a target of PRC2 regulation. FIG. 4A shows ChIP-seq data for EZH2, H3K27me3, and input at the SMN2 locus from GM12878, H1-hESCs, and HepG2 cell lines. The UCSC genome browser data from the Broad Institute shows mapped reads for EZH2, H3K27me3 and input-associated DNA along the SMN2 locus. The plot is a density graph of signal enrichment with a 25-bp overlap at any given site. FIG. 4B shows RT-qPCR for EZH1 and EZH2 in SMA fibroblast line GM09677 after EZH1 and EZH2 knockdown by transfection of their respective targeting gapmer ASO for 2 days and RT-qPCR for SMN-FL mRNA after EZH1 and EZH2 knockdown (n=2, mean+/−SD). FIG. 4C shows ChIP-qPCR data of EHZ2, H3K27me3, and total H3 from EZH1/EZH2 knockdown compared to the lipid transfection control in the SMA fibroblasts.

FIGS. 5A-5G show the identification of a novel long noncoding RNA at the SMN locus, SMN-AS1. FIG. 5A shows the mapping of SMN-AS1 positioned relative to the SMN genes. The asterisk marks the location of the C-to-T transition found in SMN2. AS3 and AS4 are northern blot probes. FIG. 5B shows a northern blot of human SMN-AS1 from human fetal brain and adult lung tissue detected with AS3 and AS4 probes (left). WT and 5025 SMA mouse brains probed with AS3 show the signal for SMN-AS1 in the 5025 mouse harboring 2 copies of the human SMN locus (right). FIG. 5C shows the correlation of expression and SMN copy number: RT-qPCR for SMN-AS1 expression levels (indicated on the left y-axis) and qPCR for the copy number levels (indicated on the right y-axis) as determined for SMA disease fibroblast lines and a carrier line by Zhong et al., 2011. GM09677 SMA fibroblasts treated with a SMN-AS1 gapmer ASO showed decreased SMN-AS1 levels. GM20384 cells lacking SMN2, but retaining SMN1, also expressed SMN-AS1. FIG. 5D shows RT-qPCR of SMN-AS1 and SMN-FL mRNA from 20 human tissue types with the fold change normalized to the expression level in the adrenal gland. FIG. 5E shows strand-specific single molecule RNA-FISH. The maximum intensity merge of widefield z-stack in GM09677 SMA fibroblasts of the nascent SMN pre-mRNA (detected by a set of intronic probes), the mature SMN mRNA, and the SMN-AS1 lncRNA are shown. Pre-mRNA signals are offset (up+left) and mature mRNA signals are offset (down+right) by 2 pixels to enable visualization. FIG. 5F shows anti-SUZ12 nRIP of SMN-AS1 with 2 primer sets (set 1 and set 2), TUG1 RNA, ANRIL RNA, 18S rRNA, GAPDH mRNA, beta-2-microglobulin (B2M), and RPL19 mRNA from SMA fibroblasts with enrichment shown as % input (mean+/−SD; n=3). IgG nRIP served as the negative control for the SUZ12 nRIP. FIG. 5G shows a RNA-EMSA of human PRC2 (EZH2/SUZ12/EED) combined with RepA I-IV, MBP (1-441), SMN-AS1 (PRC2 binding region region) or SMN-AS1 (non-binding region). The binding curves are displayed on the bottom (mean+/−S.D; n=3).

FIG. 6 shows that SMA fibroblasts transfected with Oligo 63 do not displace SMN-AS1 from the SMN locus. The maximum intensity merge of widefield z-stack in GM09677 SMA fibroblasts treated for 2 days detecting the nascent SMN mRNA transcript by probing for SMN intronic sequences (left panel), detecting SMN-AS1 (middle panel), and detecting SMN mRNA exonic sequences (right panel) are shown. The outline of the cell, the nucleus, and the probes show colocalization of SMN-AS1 with the SMN locus.

FIGS. 7A-7J show that PRC2 is associated with SMN-AS1 and that selective dissociation leads to PRC2 loss and chromatin changes at SMN locus. FIG. 7A shows a schematic diagram of the SMN2 locus with ChIP-qPCR primer positions and mixmer ASO positions. FIG. 7B shows RT-qPCR of SMN-FL mRNA after transfection with Oligo 63 and Oligo 52 in SMA fibroblasts for 2 days. FIG. 7C shows anti-SUZ12 nRIP of SMN-AS1, ANRIL, GAPDH mRNA, and 18S rRNA from SMA fibroblasts after lipid or Oligo 63 or Oligo 52 transfection; IgG RIP. (mean+/−S.D; n=3). *P<0.05 (two tailed Student's t-test). FIGS. 7D-7I show ChIP at the SMN2 locus in GM09677 SMA fibroblasts transfected with lipid, or Oligo 63 for (FIG. 7D) EZH2, (FIG. 7E) H3K27me3, (FIG. 7F) RNA Polymerase II phospho-Serine2, (FIG. 7G) H3K36me3, (FIG. 7H) pan-H3, and (FIG. 7I) H3K4me3 (mean+/−S.D; n=2). FIG. 7J shows ChIP for the promoter of HOXC13, a PRC2-regulated gene, for H3, H3K4me3, H3K36me3, RNA Polymerase II, phospho-Serine 2 (RNA PolIIpS2), H3K27me3, and EZH2 after transfection with lipid or Oligo 63 in SMA fibroblasts. (n=2, +/−SEM).

FIG. 8 shows EZH2 nRIP enrichment of SMN-AS1. The nRIP for EZH2 shows a similar pattern of enrichment of SMN-AS1 for what is observed for nRIP for SUZ12. Furthermore, EZH2 is reduced upon treatment with a steric blocking oligo, Oligo 63. GM09677 SMA fibroblasts were transfected with the steric blocking oligo Oligo 63 or an oligo targeting SMN-AS1 but not at the PRC interaction site RN-04252. Percent input for RNAs that interact with EZH2 and their resultant % input values after Oligo 63 or Oligo 52 treatment are shown. SMN-AS1, SMN-FL mRNA, ANRIL, GAPDH mRNA, and RPL19 RNAs were assessed.

FIGS. 9A-9E show upregulation of SMN expression upon Oligo 63 treatment. FIG. 9A shows RT-qPCR of SMN (exon 1-2), 47 SMN, and SMN-FL mRNA in GM09677 SMA fibroblasts (mean+/−S.D; n=5). FIG. 9B shows changes in total SMN protein levels after SMA fibroblasts were transfected with Oligo 63 for 5 days (mean+/−S.D; n=3), measured by ELISA. FIG. 9C shows western blot results for SMN and α-tubulin in SMA fibroblasts after SMA fibroblasts were transfected with Oligo 63 for 5 days (mean+/S.D; n=2). FIG. 9D shows RT-qPCR of SMN-FL mRNA in GM09677 fibroblasts that were transfected with 15 nM Oligo 63 or 15 nM SUZ12 gapmer ASO (mean+/−S.D.; n=2). *p<0.05, **p<0.01 using one-way ANOVA. A hexagonally binned scatterplot of the moderated t statistics of the 11,887 annotated genes tested for differential expression post treatment with Oligo 63 or the SUZ12 kd ASO. Each bin is colored by the number of genes that fall within it, showing the trend of Oligo 63 treated t statistics (and those less significantly differentially expressed genes) generally being reduced compared to their SUZ12 kd ASO counterpart t statistics. The Venn diagram shows the significant results (q<0.10) of the pathway analysis utilizing competitive gene set tests on 1,281 canonical pathways after treatment with each oligo. Overlap required that a pathway was both significantly in the same direction. There is significant overlap between the oligo treatments when tested with a hypergeometric test (p=1.36e⁻¹¹), however approximately 4.5 times more pathway gene sets were significantly changing with SUZ12 KD treatment. FIG. 9E shows images of untreated human SMA patient iPS-derived motor neuron cultures (left) and motor neuron cultures treated with 10 μM Oligo 63 (right) at day 11. Expression changes of SMN-FL mRNA in human SMA iPS-derived motor neuron cultures after gymnotic treatment with Oligo 63 at 20 μM for 3, 7, 9 or 11 days as a fold change from untreated cells at their respective time points (mean+/−S.D; n=2), measured by RT-qPCR (mean+/−S.D; n=2). RT-qPCR of SMN-FL mRNA in human SMA iPS-derived motor neuron cultures at day 7 after treatment with an EZH2 gapmer ASO (mean+/−S.D; n=2).

FIG. 10 shows the characterization of iPSC line and neuronal cultures representative of a SMA Type 1 patient iPSC line. Panels A-C show positive immunostaining for pluripotency markers, and panel D depicts a normal G-Band karyotype of the iPS cells. Upon neuronal induction and differentiation to the motor neuron cultures, they were found to contain: (panel E) few Nestin progenitors (<10%) and Map2 a/b neurons (dendritic marker), (panel F) pan-neurons marker β3-tubulin (>60%) with few astrolglial (GFAP) cells, and (panel G) mostly SMI32-positive motor neurons (˜40%). Scale bar for panels A-C is 75 μm. Scale bar for panels E-G is 200 μm.

FIGS. 11A-11C show that distinct mechanisms of SMN-FL mRNA generation can be complementary. FIG. 11A shows images of 5025 mouse cortical neurons at day 14 of either mock-treated or with Oligo 92 at 10 μM. FIG. 11B shows RT-qPCR of human SMN-FL mRNA relative to mouse gusb mRNA from the 5025 mouse cortical neurons treated with either 1.1, 3.3 or 10 μM Oligo 92 (mean+/−S.D; n=5). FIG. 11C shows RT-qPCR of human SMN-FL mRNA relative to mouse gusb mRNA from the 5025 mouse cortical neurons treated with 0.1, 0.3, 1.1, 3.3 or 10 μM EZH2 gapmer for 14 days (mean+/−SD; n=2).

FIGS. 12A-12J show pathway enrichment in Oligo 63 or SUZ12 kd ASO treated samples. FIG. 12A shows Reactome Double Stranded Break Repair pathway enrichment for Oligo 63. FIG. 12B shows Reactome Double Stranded Break Repair pathway enrichment for SUZ12 kd ASO. FIG. 12C shows Biocarta P53 pathway enrichment for Oligo 63. FIG. 12D shows Biocarta P53 pathway enrichment for SUZ12 kd ASO. FIG. 12E shows Reactome 3′ UTR Mediate Translational Regulation pathway enrichment for Oligo 63. FIG. 12F shows Reactome 3′ UTR Mediate Translational Regulation pathway enrichment for SUZ12 kd ASO. FIG. 12G shows Reactome Cell Cycle Mitotic pathway enrichment for Oligo 63. FIG. 12H shows Reactome Cell Cycle Mitotic pathway enrichment for SUZ12 kd ASO. FIG. 12I shows Reactome G1 S Transition pathway enrichment for Oligo 63. FIG. 12J shows Reactome G1 S Transition pathway enrichment for SUZ12 kd ASO.

### DETAILED DESCRIPTION OF CERTAIN EMBODIMENTS OF THE DISCLOSURE

Spinal muscular atrophy (SMA) is a group of hereditary diseases that causes muscle damage leading to impaired muscle function, difficulty breathing, frequent respiratory infection, and eventually death. SMA is the leading genetic cause of death in infants and children. There are four types of SMA that are classified based on the onset and severity of the disease. SMA type I is the most severe form and is one of the most common causes of infant mortality, with symptoms of muscle weakness and difficulty breathing occurring at birth. SMA type II occurs later, with muscle weakness and other symptoms developing from ages 6 months to 2 years. Symptoms appear in SMA type III during childhood and in SMA type IV, the mildest form, during adulthood. All four types of SMA have been found to be associated with mutations in the Survival of Motor Neuron (SMN) gene family, particularly SMN1.

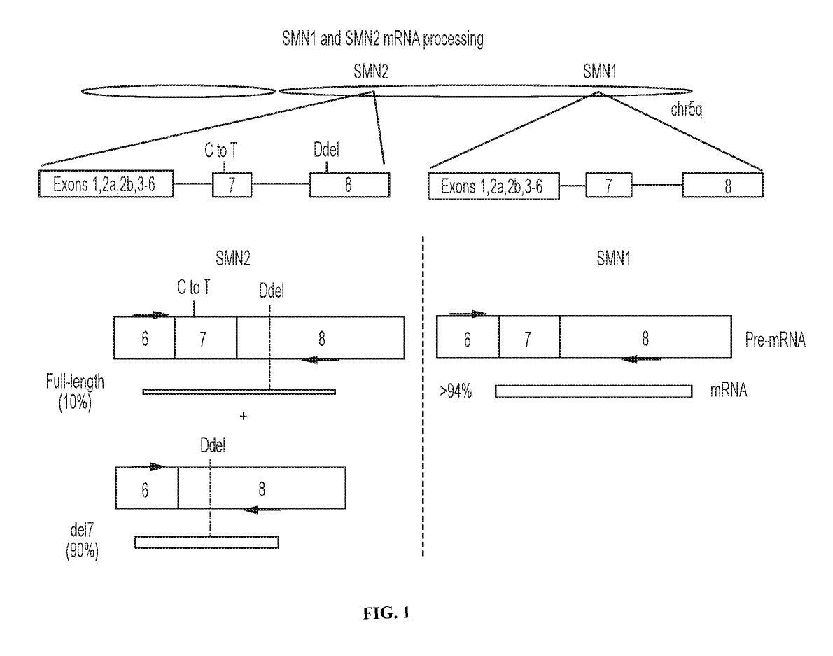

SMN protein plays a critical role in RNA splicing in motor neurons. Loss of function of the SMN1 gene is responsible for SMA. Humans have an extra SMN gene copy, called SMN2. Both SMN genes reside within a segmental duplication on Chromosome 5q13 as inverted repeats. SMN1 and SMN2 are almost identical. In some cases, SMN1 and SMN2 differ by 11 nucleotide substitutions, including seven in intron 6, two in intron 7, one in coding exon 7, and one in non-coding exon 8 (as depicted in FIG. 1.). The substitution in exon 7 involves a translationally silent C to T transition compared with SMN1, that results in alternative splicing because the substitution disrupts recognition of the upstream 3′ splice site, in which exon 7 is frequently skipped during precursor mRNA splicing. This mutation causes the inefficient splicing of SMN2 transcripts.

While most SMN1 transcripts are spliced properly, leading to the translation of a full-length protein, the majority of SMN2 transcripts lack exon 7. Consequently, SMN2 encodes primarily the exon 7-skipped protein isoform (“de17,” SMNΔ7), which is truncated protein which is unstable, mislocalized, partially functional, and rapidly degraded in cells. Therefore, the SMN2 locus leads to the expression of far less SMN protein than the SMN1 gene. SMA patients have mutations in the SMN1 gene and rely solely on the SMN2 gene for SMN protein production. It is apparent that the SMN2 gene does produce some functional SMN protein since patients lacking SMN1 but having increased DNA copy number of the SMN2 gene have a more mild disease phenotype. In addition to SMA, altered SMN expression has been implicated in other motor neuron diseases, such as Amyotrophic Lateral Sclerosis (ALS), Primary Lateral Sclerosis, Progressive Muscular Atrophy, Progressive Bulbar Palsy or Pseudobulbar Palsy.

Methods and related single stranded oligonucleotides that elevate SMN protein levels in cells (e.g., cells of a SMA patient), e.g., by increasing SMN2 transcription and correcting its splicing, are provided herein. Further aspects of the disclosure are described in detailed herein.

### Polycomb Repressive Complex 2 (PRC2)-Interacting RNAs

It has been found that, while only slightly more than 1% of the human genome is transcribed into mRNAs that encode protein, the majority of the genome is transcribed. The product of much of this transcription is long noncoding RNA (lncRNA). There are tens of thousands of distinct lncRNA expressed in the human genome. While the number of protein-encoding genes does not differ significantly from simple organisms to humans, the number and complexity of lncRNA increases dramatically with increased organismal complexity. It has been reported that there are more disease associations with lncRNA than with protein-encoding mRNA. As the name implies, lncRNAs do not encode proteins, but recent data indicate they have many other regulatory roles. lncRNA can regulate the expression of protein-encoding genes by affecting transcription, alternative splicing, and mRNA decay.

One role of lncRNA is to recruit epigenetic regulating complexes that modify chromatin to activate or repress transcription. Polycomb Repressive Complex 2 (PRC2) represses gene expression at many sites across the genome. During its transcription, a lncRNA that contains a PRC2-recognizing sequences is “tethered” to the chromosome at one end through RNA polymerase II. Because of this “tethering” to a specific chromosomal locus, the binding of the lncRNA to PRC2 usually only represses an individual neighboring mRNA. Since each PRC2-associated lncRNA interacts with PRC2 through distinct sequences it is possible to identify these sites of interaction and efficiently design synthetic oligonucleotide antagonists that specifically block the binding of PRC2 to an individual lncRNA region, thus de-repressing the expression of a single mRNA in order to produce increased amounts of the specific protein. Oligonucleotides can induce significant increases in target mRNA and protein levels without affecting neighboring non-target genes.

Aspects of the disclosure provided herein relate to the discovery of a polycomb repressive complex 2 (PRC2)-interacting RNA that inhibits expression of SMN. Polycomb repressive complex 2 (PRC2) is a histone methyltransferase and a known epigenetic regulator involved in silencing of genomic regions through methylation of histone H3. Among other functions, PRC2 interacts with long noncoding RNAs (lncRNAs), such as RepA, Xist, and Tsix, to catalyze trimethylation of histone H3-lysine27. PRC2 contains four subunits, Eed, Suz12, RbAp48, and Ezh2. Aspects of the disclosure relate to the recognition that single stranded oligonucleotides that bind to PRC2-associated regions of RNAs (e.g., lncRNAs) that are expressed from within a genomic region that encompasses or that is in functional proximity to the SMN gene can induce or enhance expression of SMN. In some embodiments, this upregulation is believed to result from inhibition of PRC2 mediated repression of SMN.

As used herein, the term “PRC2-associated region” refers to a region of a nucleic acid that comprises or encodes a sequence of nucleotides that interact directly or indirectly with a component of PRC2. A PRC2-associated region may be present in an RNA (e.g., a long non-coding RNA (lncRNA)) that interacts with PRC2. A PRC2-associated region may be present in a DNA that encodes an RNA that interacts with PRC2. In some cases, the PRC2-associated region is equivalently referred to as a PRC2-interacting region.

In some embodiments, a PRC2-associated region is a region of an RNA that crosslinks to a component of PRC2 in response to in situ ultraviolet irradiation of a cell that expresses the RNA or a region of genomic DNA that encodes that RNA region. In some embodiments, a PRC2-associated region is a region of an RNA that immunoprecipitates with an antibody that targets a component of PRC2 or a region of genomic DNA that encodes that RNA region. In some embodiments, a PRC2-associated region is a region of an RNA that immunoprecipitates with an antibody that binds specifically to SUZ12, EED, EZH2 or RBBP4 (which as noted above are components of PRC2) or a region of genomic DNA that encodes that RNA region.

In some embodiments, a PRC2-associated region is a region of an RNA that is protected from nucleases (e.g., RNases) in an RNA-immunoprecipitation assay that employs an antibody that targets a component of PRC2, or a region of genomic DNA that encodes that protected RNA region. In some embodiments, a PRC2-associated region is a region of an RNA that is protected from nucleases (e.g., RNases) in an RNA-immunoprecipitation assay that employs an antibody that targets SUZ12, EED, EZH2, or RBBP4, or a region of genomic DNA that encodes that protected RNA region.

In some embodiments, a PRC2-associated region is a region of an RNA within which occurs a relatively high frequency of sequence reads in a sequencing reaction of products of an RNA-immunoprecipitation assay that employs an antibody that targets a component of PRC2, or a region of genomic DNA that encodes that RNA region. In some embodiments, a PRC2-associated region is a region of an RNA within which occurs a relatively high frequency of sequence reads in a sequencing reaction of products of an RNA-immunoprecipitation assay that employs an antibody that binds specifically to SUZ12, EED, EZH2, or RBBP4, or a region of genomic DNA that encodes that protected RNA region. In such embodiments, the PRC2-associated region may be referred to as a “peak”.

In some embodiments, single stranded oligonucleotides are provided that specifically bind to, or are complementary to, a PRC2-associated region in a genomic region that encompasses or that is in proximity to the SMN1 or SMN2 gene.

Without being bound by a theory of disclosure, these oligonucleotides are able to interfere with the binding of and function of PRC2, by preventing recruitment of PRC2 to a specific chromosomal locus. For example, a single administration of single stranded oligonucleotides designed to specifically bind a PRC2-associated region lncRNA can stably displace not only the lncRNA, but also the PRC2 that binds to the lncRNA, from binding chromatin. After displacement, the full complement of PRC2 is not recovered for up to 24 hours. Further, lncRNA can recruit PRC2 in a cis fashion, repressing gene expression at or near the specific chromosomal locus from which the lncRNA was transcribed.

Methods of modulating gene expression are provided, in some embodiments, that may be carried out in vitro, ex vivo, or in vivo. It is understood that any reference to uses of compounds throughout the description contemplates use of the compound in preparation of a pharmaceutical composition or medicament for use in the treatment of condition (e.g., Spinal Muscular Atrophy) associated with decreased levels or activity of SMN. Thus, as one nonlimiting example, this aspect of the disclosure includes use of such single stranded oligonucleotides in the preparation of a medicament for use in the treatment of disease, wherein the treatment involves upregulating expression of SMN.

### Single Stranded Oligonucleotides for Modulating Expression of SMN

In one aspect of the disclosure, single stranded oligonucleotides complementary to PRC2-associated regions are provided for modulating expression of SMN in a cell. In some embodiments, expression of SMN is upregulated or increased. In some embodiments, single stranded oligonucleotides complementary to these PRC2-associated regions inhibit the interaction of PRC2 with long RNA transcripts such that gene expression is upregulated or increased. In some embodiments, single stranded oligonucleotides complementary to these PRC2-associated regions inhibit the interaction of PRC2 with long RNA transcripts, resulting in reduced methylation of histone H3 and reduced gene inactivation, such that gene expression is upregulated or increased. In some embodiments, this interaction may be disrupted or inhibited due to a change in the structure of the long non-coding RNA that prevents or reduces binding to PRC2.

It should be appreciated that due the high homology between SMN1 and SMN2, single stranded oligonucleotides that are complementary with a PRC2-associated region of SMN1 are often also complementary with a corresponding PRC2-associated region of SMN2.

In some embodiments, the region of complementarity of the single stranded oligonucleotide is complementary with at least 5 to 15 bases, e.g., 5, 6, 7, 8, 9, 10, 11, 12, 13, 14, or 15 consecutive nucleotides of a PRC2-associated region. In some embodiments, the region of complementarity is complementary with at least 8 (e.g. 8, 9, 10, 11, 12, 13, 14, 15, 16, 17, 18, 19, or 20) consecutive nucleotides of a PRC2-associated region. In some embodiments, a single stranded oligonucleotide may have a nucleotide sequence consisting of 8 to 14 (e.g., 8, 9, 10, 11, 12, 13 or 14) contiguous nucleotides complementary with the nucleotide sequence set forth as: ATCTGTTCCACTATG (SEQ ID NO: 1).

Complementary, as the term is used in the art, refers to the capacity for precise pairing between two nucleotides. For example, if a nucleotide at a certain position of an oligonucleotide is capable of hydrogen bonding with a nucleotide at the same position of a PRC2-associated region, then the single stranded nucleotide and PRC2-associated region are considered to be complementary to each other at that position. The single stranded nucleotide and PRC2-associated region are complementary to each other when a sufficient number of corresponding positions in each molecule are occupied by nucleotides that can hydrogen bond with each other through their bases. Thus, “complementary” is a term which is used to indicate a sufficient degree of complementarity or precise pairing such that stable and specific binding occurs between the single stranded nucleotide and PRC2-associated region. For example, if a base at one position of a single stranded nucleotide is capable of hydrogen bonding with a base at the corresponding position of a PRC2-associated region, then the bases are considered to be complementary to each other at that position. 100% complementarity is not required.

The single stranded oligonucleotide may be at least 80% complementary to (optionally one of at least 85%, 90%, 91%, 92%, 93%, 94%, 95%, 96%, 97%, 98%, 99% or 100% complementary to) the consecutive nucleotides of a PRC2-associated region. In some embodiments, the single stranded oligonucleotide may contain 1, 2, or 3 base mismatches compared to the portion of the consecutive nucleotides of a PRC2-associated region. In some embodiments the single stranded oligonucleotide may have up to 3 mismatches over 15 bases, or up to 2 mismatches over 10 bases.

It is understood in the art that a complementary nucleotide sequence need not be 100% complementary to that of its target to be specifically hybridizable. In some embodiments, a complementary nucleic acid sequence for purposes of the present disclosure is specifically hybridizable when binding of the sequence to the target molecule (e.g., lncRNA) interferes with the normal function of the target (e.g., lncRNA) to cause a loss of activity (e.g., inhibiting PRC2-associated repression with consequent up-regulation of gene expression) and there is a sufficient degree of complementarity to avoid non-specific binding of the sequence to non-target sequences under conditions in which avoidance of non-specific binding is desired, e.g., under physiological conditions in the case of in vivo assays or therapeutic treatment, and in the case of in vitro assays, under conditions in which the assays are performed under suitable conditions of stringency.

In some embodiments, the single stranded oligonucleotide is 7, 8, 9, 10, 11, 12, 13, 14, 15, 16, 17, 18, 19, 20 or more nucleotides in length. In some embodiments, the oligonucleotide is 8 to 20, 8 to 19, 8 to 18, 8 to 17, 8 to 16, 8 to 15, 9 to 20, 9 to 19, 9 to 18, 9 to 17, 9 to 16, 9 to 15, 10 to 20, 10 to 19, 10 to 18, 10 to 17, 10 to 16, or 10 to 15 nucleotides in length. In a preferred embodiment, the oligonucleotide is 8 to 15 nucleotides in length.

In some embodiments, the single stranded oligonucleotide targeting a PCR2-associated region of a long non-coding RNA that inhibits expression of SMN has a sequence set forth as CATAGTGGAACAGAT (SEQ ID NO: 14). The single stranded oligonucleotide can comprise alternating LNA nucleotides and 2′-O-methyl oligonucleotides. For example, the single stranded oligonucleotide can have a sequence set forth as CATAGTG(G)AAC(A)G(A)T (SEQ ID NO: 15), wherein the nucleotides in parenthesis are 2′O methyl (2′MOE) and all other nucleotides are LNAs. In some embodiments, the single stranded oligonucleotide has a sequence set forth as CATAGTG(G)AAC(A)G(A)T (SEQ ID NO: 16), wherein the nucleotides in parenthesis are 2′O methyl (2′MOE) and all other nucleotides are LNAs, while the underlined nucleotides are 5-methylcytosines.

In some embodiments, any one or more thymidine (T) nucleotides (or modified nucleotide thereof) or uridine (U) nucleotides (or a modified nucleotide thereof) in a sequence provided herein, including a sequence provided in the sequence listing, may be replaced with any other nucleotide suitable for base pairing (e.g., via a Watson-Crick base pair) with an adenosine nucleotide. In some embodiments, any one or more thymidine (T) nucleotides (or modified nucleotide thereof) or uridine (U) nucleotides (or a modified nucleotide thereof) in a sequence provided herein, including a sequence provided in the sequence listing, may be suitably replaced with a different pyrimidine nucleotide or vice versa. In some embodiments, any one or more thymidine (T) nucleotides (or modified nucleotide thereof) in a sequence provided herein, including a sequence provided in the sequence listing, may be suitably replaced with a uridine (U) nucleotide (or a modified nucleotide thereof) or vice versa. In some embodiments, GC content of the single stranded oligonucleotide is preferably between about 30-60%. Contiguous runs of three or more Gs or Cs may not be preferable in some embodiments. Accordingly, in some embodiments, the oligonucleotide does not comprise a stretch of three or more guanosine nucleotides.

In some embodiments, the single stranded oligonucleotide specifically binds to, or is complementary to an RNA that is encoded in a genome (e.g., a human genome) as a single contiguous transcript (e.g., a non-spliced RNA).

In some embodiments, single stranded oligonucleotides disclosed herein may increase expression of mRNA corresponding to the gene by at least about 50% (e.g., 150% of normal or 1.5 fold), or by about 2 fold to about 5 fold. In some embodiments, expression may be increased by at least about 15 fold, 20 fold, 30 fold, 40 fold, 50 fold or 100 fold, or any range between any of the foregoing numbers. It has also been found that increased mRNA expression has been shown to correlate to increased protein levels.

### Splice Correcting Agents

Aspects of the disclosure provide methods for targeting SMN precursor mRNA to affect splicing to minimize or prevent exon skipping. In some embodiments, agents (e.g., small molecules, oligonucleotides) are provided herein that modulate SMN2 splicing. Such agents are referred to herein as “splice correcting agents.”

In some embodiments, methods are provided that involve delivering to a cell i) a single stranded oligonucleotide complementary with at least 8 (e.g., 8 to 15) consecutive nucleotides of a PRC2-associated region of an lncRNA expressed from the SMN2 gene locus and ii) a splice correcting agent. In this context, oligonucleotides targeting PRC2-associated regions may be referred to herein as “transcriptional oligonucleotides” because they affect SMN transcription (e.g., by relieving PRC2-mediated repression) as compared with splice correcting agents which effect splicing.

In some embodiments, splice correcting agents may be in the form of oligonucleotides, referred to herein as “splice correcting oligonucleotides,” that modulate SMN2 splicing. Splice correcting oligonucleotides typically comprise a sequence complementary to a splice control sequence (e.g., a intronic splicing silencer sequence) of a precursor mRNA, and are capable of binding to and affecting processing of the precursor mRNA. Splice correcting oligonucleotides may be complementary with a region of an exon, a region of an intron or an intron/exon junction. In some embodiments, the splice control sequence comprises the sequence: GUAAGUCUGCCAGCAUUAUGAAAG (SEQ ID NO: 2) or the sequence CAGCAUUAUGAAAG (SEQ ID NO: 5) or a portion of either one. In some embodiments, the splice correcting oligonucleotide is complementary with or contains a region that is complementary with at least 8 (e.g., 8 to 15) consecutive nucleotides of a splice control sequence, e.g., SEQ ID NO: 2, SEQ ID NO: 5, or a portion thereof.

In some embodiments, the splice control sequence comprises at least one hnRNAP binding sequence. In some embodiments, splice correcting oligonucleotides that target SMN2 function based on the premise that there is a competition between the 3′ splice sites of exons 7 and 8 for pairing with the 5′ splice site of exon 6, so impairing the recognition of the 3′ splice site of exon 8 favors exon 7 inclusion. In some embodiments, splice correcting oligonucleotides are provided that promote SMN2 exon 7 inclusion and full-length SMN protein expression, in which the oligonucleotides are complementary to the intron 7/exon 8 junction. In some embodiments, splice correcting oligonucleotides are composed of a segment complementary to an exon of SMN (e.g., exon 7). In some embodiments, splice correcting oligonucleotides comprise a tail (e.g., a non-complementary tail) consisting of RNA sequences with binding motifs recognized by a serine/arginine-rich (SR) protein. In some embodiments, splice correcting oligonucleotides are complementary (at least partially) with an intronic splicing silencer (ISS). In some embodiments, the ISS is in intron 6 or intron 7 of SMN1 or SMN2. In some embodiments, splice correcting oligonucleotides comprise an antisense moiety complementary to a target exon or intron (e.g., of SMN1 or SMN2) and a minimal RS domain peptide similar to the splicing activation domain of SR proteins. In some embodiments, the splice correcting oligonucleotide is 7, 8, 9, 10, 11, 12, 13, 14, 15, 16, 17, 18, 19, 20, 21, 22, 23, 24, 25, 26, 27, 28, 29, 30, 35, 40, 45, 50, or more nucleotides in length. In some embodiments, the oligonucleotide is 8 to 20, 8 to 19, 8 to 18, 8 to 17, 8 to 16, 8 to 15, 9 to 20, 9 to 19, 9 to 18, 9 to 17, 9 to 16, 9 to 15, 10 to 20, 10 to 19, 10 to 18, 10 to 17, 10 to 16, or 10 to 15 nucleotides in length. In one embodiment, the oligonucleotide is 8 to 15 nucleotides in length.

In some embodiments, the splice correcting oligonucleotide has a sequence set forth as TCACTTTCATAATGC (SEQ ID NO: 17). The splice correcting oligonucleotide can comprise alternating LNA nucleotides and 2′-O-methyl oligonucleotides. For example, the splice correcting oligonucleotide can have a sequence set forth as TCACTTT(C)ATA(A)T(G)C (SEQ ID NO: 18), wherein the nucleotides in parenthesis are 2′O methyl (2′MOE) and all other nucleotides are LNAs. In some embodiments, at least some of the cytosine nucleotides in the splice correcting oligonucleotide are 5-methylcytosines. For example, the splice correcting oligonucleotide can have a sequence set forth as TCACTTT(C)ATA(A)T(G)C (SEQ ID NO: 19), wherein the nucleotides in parenthesis are 2′O methyl (2′MOE) and all other nucleotides are LNAs, while the underlined nucleotides are 5-methylcytosines.

In some embodiments, the splice correcting oligonucleotide has a sequence set forth as ACTTTCATAATGCTGG (SEQ ID NO: 20). The splice correcting oligonucleotide can comprise alternating LNA nucleotides and 2′-O-methyl oligonucleotides. For example, the splice correcting oligonucleotide can have a sequence set forth as ACTTTCAT(A)ATG(C)T(G)G (SEQ ID NO: 21), wherein the nucleotides in parenthesis are 2′O methyl (2′MOE) and all other nucleotides are LNAs. In some embodiments, at least some of the cytosine nucleotides in the splice correcting oligonucleotide are 5-methylcytosines. For example, the splice correcting oligonucleotide can have a sequence set forth as ACTTTCAT(A)ATG(C)T(G)G (SEQ ID NO: 22), wherein the nucleotides in parenthesis are 2′O methyl (2′MOE) and all other nucleotides are LNAs, while the underlined nucleotides are 5-methylcytosines.

In some embodiments, the splice correcting oligonucleotide has a sequence set forth as GCTGGCAG. In some embodiments, the splice correcting oligonucleotide comprises or consists of 2′O-methyl oligonucleotides. In some embodiments, the splice correcting oligonucleotide comprises or consists of LNA nucleotides. In some embodiments, the cytosines in the splice correcting oligonucleotide are 5-methylcytosines.

In some embodiments, the splice correcting oligonucleotide has a sequence as disclosed in the U.S. Pat. Nos. 7,033,752; 7,838,657; 8,110,560; 8,361,977; 8,586,559; 8,946,183; and 8,980,853; a sequence as disclosed in the United States Patent Application Nos. US 2014/0357558; and US2012/0190728; or a sequence as disclosed in the International Patent Publication Nos.: WO 2012/178146 and WO 2010/148249, each of which is herein incorporated by reference.

In some embodiments, the splice correcting oligonucleotide has a sequence as disclosed in Singh et al., 2006 Mol Cell Biol. 26(4):1333-46; Singh et al., 2009 RNA Biol. 6(3):341-50; or Hua et al., 2007 PLoS Biol. 5(4):e73.

In some embodiments, splice correcting agents may be in the form of small molecules, referred to herein as “splice correcting small molecules,” that modulate SMN2 splicing.

### Linkers

Any of the oligonucleotides disclosed herein may be linked to one or more other oligonucleotides or small molecules (e.g., small molecules that function as splice correcting agents) disclosed herein by a linker, e.g., a cleavable linker. Accordingly, in some embodiments, compounds are provided that comprise an oligonucleotide complementary with a PRC2-associated region of a gene that is linked via a linker to a splice correcting agent (e.g., an oligonucleotide complementary to a splice control sequence of a precursor mRNA of the gene). In some embodiments, compounds are provided that have the general formula A-B-C, in which A is an oligonucleotide complementary with a PRC2-associated region of a gene, B is a linker, and C is a splice correcting agent (e.g., a single stranded oligonucleotide complementary to a splice control sequence of a precursor mRNA of the gene). In some embodiments, linker B comprises an oligonucleotide, peptide, low pH labile bond, or disulfide bond. In some embodiments, the compound comprises oligonucleotide A and oligonucleotide C and is orientated as 5′-A-B-C-3′. In some embodiments, the compound comprises oligonucleotide A and oligonucleotide C and is orientated as 3′-A-B-C-5′. In some embodiments, where B is an oligonucleotide, the 3′ end of A is linked to the 5′ end of B, and the 3′ end of B is linked to 5′ end of C. In some embodiments, where B is an oligonucleotide, the 5′ end of A is linked to the 3′ end of B, and the 5′ end of B is linked to 3′ end of C. In some embodiments, where B is an oligonucleotide, the 5′ end of A is linked to the 5′ end of B, and/or the 3′ end of B is linked to the 3′ end of C. In some embodiments, where B is an oligonucleotide, the 3′ end of A is linked to the 3′ end of B, and/or the 5′ end of B is linked to the 5′ end of C.

The term “linker” generally refers to a chemical moiety that is capable of covalently linking two or more oligonucleotides. In some embodiments, at least one bond comprised or contained within the linker is capable of being cleaved (e.g., in a biological context, such as in a mammalian extract, such as an endosomal extract), such that at least two oligonucleotides are no longer covalently linked to one another after bond cleavage. It will be appreciated that, in some embodiments, a provided linker may include a region that is non-cleavable, as long as the linker also comprises at least one bond that is cleavable.

In some embodiments, the linker is an oligonucleotide linker that comprises a sequence set forth as Wₙ, wherein W is a nucleotide selected from A, T, and U, and n is a integer selected from 2, 3, and 4, representing the number of instances of W.

In some embodiments, the linker comprises a polypeptide that is more susceptible to cleavage by an endopeptidase in the mammalian extract than the oligonucleotides. The endopeptidase may be a trypsin, chymotrypsin, elastase, thermolysin, pepsin, or endopeptidase V8. The endopeptidase may be a cathepsin B, cathepsin D, cathepsin L, cathepsin C, papain, cathepsin S, or endosomal acidic insulinase. For example, the linker may comprise a peptide having an amino acid sequence selected from: ALAL (SEQ ID NO: 8), APISFFELG (SEQ ID NO: 9), FL, GFN, R/KXX, GRWHTVGLRWE (SEQ ID NO: 10), YL, GF, and FF, in which X is any amino acid.

In some embodiments, the linker comprises the formula —(CH₂)ₙS—S(CH₂)ₘ—, wherein n and m are independently integers from 0 to 10.

In some embodiments, the linker may comprise an oligonucleotide that is more susceptible to cleavage by an endonuclease in the mammalian extract than the oligonucleotides. The linker may have a nucleotide sequence comprising from 1 to 10 (e.g., 1, 2, 3, 4, 5, 6, 7, 8, 9 or 10) pyrimidines, such as thymidines or uridines, linked through phosphodiester internucleotide linkages. The linker may have a nucleotide sequence comprising deoxyribonucleotides linked through phosphodiester internucleotide linkages. The linker may have a nucleotide sequence comprising from 1 to 10 thymidines or uridines linked through phosphodiester internucleotide linkages. The linker may have a nucleotide sequence comprising from 1 to 10 e.g., 1, 2, 3, 4, 5, 6, 7, 8, 9 or 10) pyrimidines, such as thymidines or uridines, linked through phosphorothioate internucleotide linkages.

In some embodiments, at least one linker is 2-fold, 3-fold, 4-fold, 5-fold, 10-fold or more sensitive to enzymatic cleavage in the presence of a mammalian extract than at least two oligonucleotides. It should be appreciated that different linkers can be designed to be cleaved at different rates and/or by different enzymes in compounds comprising two or more linkers. Similarly different linkers can be designed to be sensitive to cleavage in different tissues, cells or subcellular compartments in compounds comprising two or more linkers. This can advantageously permit compounds to have oligonucleotides that are released from compounds at different rates, by different enzymes, or in different tissues, cells or subcellular compartments thereby controlling release of the monomeric oligonucleotides to a desired in vivo location or at a desired time following administration.

In certain embodiments, linkers are stable (e.g., more stable than the oligonucleotides they link together) in plasma, blood, or serum which are richer in exonucleases, and less stable in the intracellular environments which are relatively rich in endonucleases. In some embodiments, a linker is considered “non-cleavable” if the linker's half-life is at least 24, or 28, 32, 36, 48, 72, 96 hours, or longer under the conditions described here, such as in liver homogenates. Conversely, in some embodiments, a linker is considered “cleavable” if the half-life of the linker is at most 10, or 8, 6, 5 hours, or shorter.

In some embodiments, the linker is a nuclease-cleavable oligonucleotide linker. In some embodiments, the nuclease-cleavable linker contains one or more phosphodiester bonds in the oligonucleotide backbone. For example, the linker may contain a single phosphodiester bridge or 2, 3, 4, 5, 6, 7, or more phosphodiester linkages, for example as a string of 1-10 deoxynucleotides, e.g., dT, or ribonucleotides, e.g., rU, in the case of RNA linkers. In the case of dT or other DNA nucleotides dN (e.g., dA) in the linker, in certain embodiments, the cleavable linker contains one or more phosphodiester linkages. In other embodiments, in the case of rU or other RNA nucleotides rN, the cleavable linker may consist of phosphorothioate linkages only. In contrast to phosphorothioate-linked deoxynucleotides, which in some embodiments are cleaved relatively slowly by nucleases (thus termed “noncleavable”), phosphorothioate-linked rU undergoes relatively rapid cleavage by ribonucleases and therefore is considered cleavable herein in some embodiments. It is also possible to combine dN and rN into the linker region, which are connected by phosphodiester or phosphorothioate linkages. In other embodiments, the linker can also contain chemically modified nucleotides, which are still cleavable by nucleases, such as, e.g., 2′-O-modified analogs. In particular, 2′-O-methyl or 2′-fluoro nucleotides can be combined with each other or with dN or rN nucleotides. Generally, in the case of nucleotide linkers, the linker is a part of the compound that is usually not complementary to a target, although it could be. This is because the linker is generally cleaved prior to action of the oligonucleotides on the target, and therefore, the linker identity with respect to a target is inconsequential. Accordingly, in some embodiments, a linker is an (oligo)nucleotide linker that is not complementary to any of the targets against which the oligonucleotides are designed.

In some embodiments, the cleavable linker is an oligonucleotide linker that contains a continuous stretch of deliberately introduced Rp phosphorothioate stereoisomers (e.g., 4, 5, 6, 7, or longer stretches). The Rp stereoisoform, unlike Sp isoform, is known to be susceptible to nuclease cleavage (Krieg et al., 2003, Oligonucleotides, 13:491-499). Such a linker would not include a racemic mix of PS linkaged oligonucleotides since the mixed linkages are relatively stable and are not likely to contain long stretches of the Rp stereoisomers, and therefore, considered “non-cleavable” herein. Thus, in some embodiments, a linker comprises a stretch of 4, 5, 6, 7, or more phosphorothioated nucleotides, wherein the stretch does not contain a substantial amount or any of the Sp stereoisoform. The amount could be considered substantial if it exceeds 10% on a per-mole basis.

In some embodiments, the linker is a non-nucleotide linker, for example, a single phosphodiester bridge. Another example of such cleavable linkers is a chemical group comprising a disulfide bond, for example, —(CH₂)—S—S(CH₂)ₘ—, wherein n and m are integers from 0 to 10. In illustrative embodiments, n=m=6. Additional examples of non-nucleotide linkers are described below.

The linker can be designed so as to undergo a chemical or enzymatic cleavage reaction. Chemical reactions involve, for example, cleavage in acidic environments (e.g., endosomes), reductive cleavage (e.g., cytosolic cleavage) or oxidative cleavage (e.g., in liver microsomes). The cleavage reaction can also be initiated by a rearrangement reaction. Enzymatic reactions can include reactions mediated by nucleases, peptidases, proteases, phosphatases, oxidases, reductases, etc. For example, a linker can be pH-sensitive, cathepsin-sensitive, or predominantly cleaved in endosomes and/or cytosol.

In some embodiments, the linker comprises a peptide. In certain embodiments, the linker comprises a peptide which includes a sequence that is cleavable by an endopeptidase. In addition to the cleavable peptide sequence, the linker may comprise additional amino acid residues and/or non-peptide chemical moieties, such as an alkyl chain. In certain embodiments, the linker comprises Ala-Leu-Ala-Leu (SEQ ID NO: 8), which is a substrate for cathepsin B. See, for example, the maleimidocaproyl-Arg-Arg-Ala-Leu-Ala-Leu (SEQ ID NO: 11) linkers described in Schmid et al, Bioconjugate Chem 2007, 18, 702-716. In certain embodiments, a cathepsin B-cleavable linker is cleaved in tumor cells. In certain embodiments, the linker comprises Ala-Pro-Ile-Ser-Phe-Phe-Glu-Leu-Gly (SEQ ID NO: 9), which is a substrate for cathepsins D, L, and B (see, for example, Fischer et al, Chembiochem 2006, 7, 1428-1434). In certain embodiments, a cathepsin-cleavable linker is cleaved in HeLA cells. In some embodiments, the linker comprises Phe-Lys, which is a substrate for cathepsin B. For example, in certain embodiments, the linker comprises Phe-Lys-p-aminobenzoic acid (PABA). See, e.g., the maleimidocaproyl-Phe-Lys-PABA linker described in Walker et al., Bioorg. Med. Chem. Lett. 2002, 12, 217-219. In certain embodiments, the linker comprises Gly-Phe-2-naphthylamide, which is a substrate for cathepsin C (see, for example, Berg et al. Biochem. J. 1994, 300, 229-235). In certain embodiments, a cathepsin C-cleavable linker is cleaved in hepatocytes. In some embodiments, the linker comprises a cathepsin S cleavage site. For example, in some embodiments, the linker comprises Gly-Arg-Trp-His-Thr-Val-Gly-Leu-Arg-Trp-Glu (SEQ ID NO: 10), Gly-Arg-Trp-Pro-Pro-Met-Gly-Leu-Pro-Trp-Glu (SEQ ID NO: 12), or Gly-Arg-Trp-His-Pro-Met-Gly-Ala-Pro-Trp-Glu (SEQ ID NO: 13, for example, as described in Lutzner et al., J. Biol. Chem. 2008, 283, 36185-36194. In certain embodiments, a cathepsin S-cleavable linker is cleaved in antigen presenting cells. In some embodiments, the linker comprises a papain cleavage site. Papain typically cleaves a peptide having the sequence—R/K-X-X (see Chapman et al., Annu. Rev. Physiol 1997, 59, 63-88). In certain embodiments, a papain-cleavable linker is cleaved in endosomes. In some embodiments, the linker comprises an endosomal acidic insulinase cleavage site. For example, in some embodiments, the linker comprises Tyr-Leu, Gly-Phe, or Phe-Phe (see, e.g., Authier et al, FEBS Lett. 1996, 389, 55-60). In certain embodiments, an endosomal acidic insulinase-cleavable linker is cleaved in hepatic cells.

In some embodiments, the linker is pH sensitive. In certain embodiments, the linker comprises a low pH-labile bond. As used herein, a low-pH labile bond is a bond that is selectively broken under acidic conditions (pH<7). Such bonds may also be termed endosomally labile bonds, because cell endosomes and lysosomes have a pH less than 7. For example, in certain embodiments, the linker comprises an amine, an imine, an ester, a benzoic imine, an amino ester, a diortho ester, a polyphosphoester, a polyphosphazene, an acetal, a vinyl ether, a hydrazone, an azidomethyl-methylmaleic anhydride, a thiopropionate, a masked endosomolytic agent, or a citraconyl group.

In certain embodiments, the linker comprises a low pH-labile bond selected from the following: ketals that are labile in acidic environments (e.g., pH less than 7, greater than about 4) to form a diol and a ketone; acetals that are labile in acidic environments (e.g., pH less than 7, greater than about 4) to form a diol and an aldehyde; imines or iminiums that are labile in acidic environments (e.g., pH less than 7, greater than about 4) to form an amine and an aldehyde or a ketone; silicon-oxygen-carbon linkages that are labile under acidic condition; silicon-nitrogen (silazane) linkages; silicon-carbon linkages (e.g., arylsilanes, vinylsilanes, and allylsilanes); maleamates (amide bonds synthesized from maleic anhydride derivatives and amines); ortho esters; hydrazones; activated carboxylic acid derivatives (e.g., esters, amides) designed to undergo acid catalyzed hydrolysis); or vinyl ethers.

In some embodiments, the linker comprises a masked endosomolytic agent. Endosomolytic polymers are polymers that, in response to a change in pH, are able to cause disruption or lysis of an endosome or provide for escape of a normally membrane-impermeable compound, such as a polynucleotide or protein, from a cellular internal membrane-enclosed vesicle, such as an endosome or lysosome. A subset of endosomolytic compounds is fusogenic compounds, including fusogenic peptides. Fusogenic peptides can facilitate endosomal release of agents such as oligomeric compounds to the cytoplasm. See, for example, US Patent Application Publication Nos. 20040198687, 20080281041, 20080152661, and 20090023890, which are incorporated herein by reference.

The linker can also be designed to undergo an organ/tissue-specific cleavage. For example, for certain targets, which are expressed in multiple tissues, only the knock-down in liver may be desirable, as knock-down in other organs may lead to undesired side effects. Thus, linkers susceptible to liver-specific enzymes, such as pyrrolase (TPO) and glucose-6-phosphatase (G-6-Pase), can be engineered, so as to limit the antisense effect to the liver mainly. Alternatively, linkers not susceptible to liver enzymes but susceptible to kidney-specific enzymes, such as gamma-glutamyltranspeptidase, can be engineered, so that the antisense effect is limited to the kidneys mainly. Analogously, intestine-specific peptidases cleaving Phe-Ala and Leu-Ala could be considered for orally administered multimeric oligonucleotides. Similarly, by placing an enzyme recognition site into the linker, which is recognized by an enzyme over-expressed in tumors, such as plasmin (e.g., PHEA-D-Val-Leu-Lys recognition site), tumor-specific knock-down should be feasible. By selecting the right enzyme recognition site in the linker, specific cleavage and knock-down should be achievable in many organs. In addition, the linker can also contain a targeting signal, such as N-acetyl galactosamine for liver targeting, or folate, vitamin A or RGD-peptide in the case of tumor or activated macrophage targeting. Accordingly, in some embodiments, the cleavable linker is organ- or tissue-specific, for example, liver-specific, kidney-specific, intestine-specific, etc.

The oligonucleotides can be linked through any part of the individual oligonucleotide, e.g., via the phosphate, the sugar (e.g., ribose, deoxyribose), or the nucleobase. In certain embodiments, when linking two oligonucleotides together, the linker can be attached e.g., to the 5′-end of the first oligonucleotide and the 3′-end of the second nucleotide, to the 5′-end of the first oligonucleotide and the 5′end of the second nucleotide, to the 3′-end of the first oligonucleotide and the 3′-end of the second nucleotide. In other embodiments, when linking two oligonucleotides together, the linker can attach internal residues of each oligonucleotides, e.g., via a modified nucleobase. One of ordinary skill in the art will understand that many such permutations are available for multimers. Further examples of appropriate linkers as well as methods for producing compounds having such linkers are disclosed in International Patent Application Number, PCT/US2012/055535, entitled MULTIMERIC OLIGONUCLEOTIDE COMPOUNDS, publication number WO2013040429 A1 and International Patent Application Number PCT/US2013/59772, entitled MULTIMERIC OLIGONUCLEOTIDE COMPOUNDS, publication number WO2014/043544, the contents of each of which relating to linkers and related chemistries are incorporated herein by reference in their entireties.

### Compounds for Modulating Expression of SMN

In one aspect of the disclosure, compounds that increase SMN2 transcription and correct its splicing are provided herein. In some embodiments, such compound comprises a single stranded oligonucleotide complementary to a PRC2-associated region described herein and a splice correcting agent described herein, wherein the single stranded oligonucleotide and the splice correcting agent are linked by a linker described herein. In some embodiments, the single stranded oligonucleotide and the splice correcting agent are linked by a covalent linker.

In some embodiments, the compound comprises a single stranded oligonucleotide complementary to a PRC2-associated region and a splice correcting oligonucleotide described herein, wherein the single stranded oligonucleotide and the splice correcting oligonucleotide are covalently linked, e.g., by an oligonucleotide linker (e.g., a DNA linker). Non-limiting examples of such compound include, but are not limited to CATAGTG(G)AAC(A)G(A)ToAoAo(G)(C)(U)(G)(G)(C)(A)(G) (SEQ ID NO: 23); CATAGTG(G)AAC(A)G(A)ToAoAoAo (G)(C)(U)(G)(G)(C)(A)(G) (SEQ ID NO: 24); and CATAGTG(G)AAC(A)G(A)ToAoAoAoGCTGGCAG (SEQ ID NO: 25). Nucleotides in parenthesis are 2′O methyl (2′MOE) and all other nucleotides are LNAs. Underlined nucleotides are 5-methylcytosines. The nucleotides that are in bold correspond to the nucleotides of a linker between a single stranded oligonucleotide complementary to a PCR2-associated region and a splice correcting oligonucleotide. The symbol “o” corresponds to a phosphodiester bond between two nucleotides in a linker or a phosphodiester bond linking one end of a linker to a single stranded oligonucleotide complementary to a PRC2-associated region or a splice correcting oligonucleotide. Without wishing to be limited, other oligonucleotide linkers can be used in place of “AA” or “AAA” as disclosed in SEQ ID Nos. 23 to 25.

In some embodiments, the compound comprises a single stranded oligonucleotide complementary to a PRC2-associated region and a splice correcting oligonucleotide having a sequence as disclosed in the U.S. Pat. Nos. 7,033,752; 7,838,657; 8,110,560; 8,361,977; 8,586,559; 8,946,183; and 8,980,853; a sequence as disclosed in the United States Patent Application Nos. US 2014/0357558; and US2012/0190728; or a sequence as disclosed in the International Patent Publication Nos.: WO 2012/178146 and WO 2010/148249, each of which is herein incorporated by reference.

### Nucleotide Modifications

In some embodiments, the oligonucleotide may comprise at least one ribonucleotide, at least one deoxyribonucleotide, and/or at least one bridged nucleotide. In some embodiments, the oligonucleotide may comprise a bridged nucleotide, such as a locked nucleic acid (LNA) nucleotide, a constrained ethyl (cEt) nucleotide, or an ethylene bridged nucleic acid (ENA) nucleotide. Examples of such nucleotides are disclosed herein and known in the art. In some embodiments, the oligonucleotide comprises a nucleotide analog disclosed in one of the following United States Patent or Patent Application Publications: U.S. Pat. No. 7,399,845, U.S. Pat. No. 7,741,457, U.S. Pat. No. 8,022,193, U.S. Pat. No. 7,569,686, U.S. Pat. No. 7,335,765, U.S. Pat. No. 7,314,923, U.S. Pat. No. 7,335,765, and U.S. Pat. No. 7,816,333, US 20110009471, the entire contents of each of which are incorporated herein by reference for all purposes. The oligonucleotide may have one or more 2′ O-methyl nucleotides. The oligonucleotide may consist entirely of 2′ O-methyl nucleotides.

Often the single stranded oligonucleotide has one or more nucleotide analogues. For example, the single stranded oligonucleotide may have at least one nucleotide analogue that results in an increase in Tₘ of the oligonucleotide in a range of 1° C., 2° C., 3° C., 4° C., or 5° C. compared with an oligonucleotide that does not have the at least one nucleotide analogue. The single stranded oligonucleotide may have a plurality of nucleotide analogues that results in a total increase in Tₘ of the oligonucleotide in a range of 2° C., 3° C., 4° C., 5° C., 6° C., 7° C., 8° C., 9° C., 10° C., 15° C., 20° C., 25° C., 30° C., 35° C., 40° C., 45° C., or more compared with an oligonucleotide that does not have the nucleotide analogue.

The oligonucleotide may be of up to 50 nucleotides in length in which 2 to 10, 2 to 15, 2 to 16, 2 to 17, 2 to 18, 2 to 19, 2 to 20, 2 to 25, 2 to 30, 2 to 40, 2 to 45, or more nucleotides of the oligonucleotide are nucleotide analogues. The oligonucleotide may be of 8 to 30 nucleotides in length in which 2 to 10, 2 to 15, 2 to 16, 2 to 17, 2 to 18, 2 to 19, 2 to 20, 2 to 25, 2 to 30 nucleotides of the oligonucleotide are nucleotide analogues.

The oligonucleotide may be of 8 to 15 nucleotides in length in which 2 to 4, 2 to 5, 2 to 6, 2 to 7, 2 to 8, 2 to 9, 2 to 10, 2 to 11, 2 to 12, 2 to 13, 2 to 14 nucleotides of the oligonucleotide are nucleotide analogues. Optionally, the oligonucleotides may have every nucleotide except 1, 2, 3, 4, 5, 6, 7, 8, 9, or 10 nucleotides modified.

The oligonucleotide may consist entirely of bridged nucleotides (e.g., LNA nucleotides, cEt nucleotides, ENA nucleotides). The oligonucleotide may comprise alternating deoxyribonucleotides and 2′-fluoro-deoxyribonucleotides. The oligonucleotide may comprise alternating deoxyribonucleotides and 2′-O-methyl nucleotides. The oligonucleotide may comprise alternating deoxyribonucleotides and ENA nucleotide analogues. The oligonucleotide may comprise alternating deoxyribonucleotides and LNA nucleotides. The oligonucleotide may comprise alternating LNA nucleotides and 2′-O-methyl nucleotides. The oligonucleotide may have a 5′ nucleotide that is a bridged nucleotide (e.g., a LNA nucleotide, cEt nucleotide, ENA nucleotide). The oligonucleotide may have a 5′ nucleotide that is a deoxyribonucleotide.

The oligonucleotide may comprise deoxyribonucleotides flanked by at least one bridged nucleotide (e.g., a LNA nucleotide, cEt nucleotide, ENA nucleotide) on each of the 5′ and 3′ ends of the deoxyribonucleotides. The oligonucleotide may comprise deoxyribonucleotides flanked by 1, 2, 3, 4, 5, 6, 7, 8 or more bridged nucleotides (e.g., LNA nucleotides, cEt nucleotides, ENA nucleotides) on each of the 5′ and 3′ ends of the deoxyribonucleotides. The 3′ position of the oligonucleotide may have a 3′ hydroxyl group. The 3′ position of the oligonucleotide may have a 3′ thiophosphate.

The oligonucleotide may be conjugated with a label. For example, the oligonucleotide may be conjugated with a biotin moiety, cholesterol, Vitamin A, folate, sigma receptor ligands, aptamers, peptides, such as CPP, hydrophobic molecules, such as lipids, ASGPR or dynamic polyconjugates and variants thereof at its 5′ or 3′ end.

Preferably the single stranded oligonucleotide comprises one or more modifications comprising: a modified sugar moiety, and/or a modified internucleoside linkage, and/or a modified nucleotide and/or combinations thereof. It is not necessary for all positions in a given oligonucleotide to be uniformly modified, and in fact more than one of the modifications described herein may be incorporated in a single oligonucleotide or even at within a single nucleoside within an oligonucleotide.

In some embodiments, the single stranded oligonucleotides are chimeric oligonucleotides that contain two or more chemically distinct regions, each made up of at least one nucleotide. These oligonucleotides typically contain at least one region of modified nucleotides that confers one or more beneficial properties (such as, for example, increased nuclease resistance, increased uptake into cells, increased binding affinity for the target) and a region that is a substrate for enzymes capable of cleaving RNA:DNA or RNA:RNA hybrids. Chimeric single stranded oligonucleotides of the disclosure may be formed as composite structures of two or more oligonucleotides, modified oligonucleotides, oligonucleosides and/or oligonucleotide mimetics as described above. Such compounds have also been referred to in the art as hybrids or gapmers. Representative United States patents that teach the preparation of such hybrid structures comprise, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922, each of which is herein incorporated by reference.

In some embodiments, the single stranded oligonucleotide comprises at least one nucleotide modified at the 2′ position of the sugar, most preferably a 2′-O-alkyl, 2′-O-alkyl-O-alkyl or 2′-fluoro-modified nucleotide. In other preferred embodiments, RNA modifications include 2′-fluoro, 2′-amino and 2′ O-methyl modifications on the ribose of pyrimidines, abasic residues or an inverted base at the 3′ end of the RNA. Such modifications are routinely incorporated into oligonucleotides and these oligonucleotides have been shown to have a higher Tm (e.g., higher target binding affinity) than 2′-deoxyoligonucleotides against a given target.

A number of nucleotide and nucleoside modifications have been shown to make the oligonucleotide into which they are incorporated more resistant to nuclease digestion than the native oligodeoxynucleotide; these modified oligos survive intact for a longer time than unmodified oligonucleotides. Specific examples of modified oligonucleotides include those comprising modified backbones, for example, phosphorothioates, phosphotriesters, methyl phosphonates, short chain alkyl or cycloalkyl intersugar linkages or short chain heteroatomic or heterocyclic intersugar linkages. Most preferred are oligonucleotides with phosphorothioate backbones and those with heteroatom backbones, particularly CH₂—NH—O—CH₂, CH, ˜N(CH₃)˜O˜CH₂ (known as a methylene(methylimino) or MMI backbone, CH₂—O—N(CH₃)—CH₂, CH₂—N(CH₃)—N(CH₃)—CH₂ and O—N(CH₃)—CH₂—CH₂ backbones, wherein the native phosphodiester backbone is represented as O—P—O—CH); amide backbones (see De Mesmaeker et al. Ace. Chem. Res. 1995, 28:366-374); morpholino backbone structures (see Summerton and Weller, U.S. Pat. No. 5,034,506); peptide nucleic acid (PNA) backbone (wherein the phosphodiester backbone of the oligonucleotide is replaced with a polyamide backbone, the nucleotides being bound directly or indirectly to the aza nitrogen atoms of the polyamide backbone, see Nielsen et al., Science 1991, 254, 1497). Phosphorus-containing linkages include, but are not limited to, phosphorothioates, chiral phosphorothioates, phosphorodithioates, phosphotriesters, aminoalkylphosphotriesters, methyl and other alkyl phosphonates comprising 3′alkylene phosphonates and chiral phosphonates, phosphinates, phosphoramidates comprising 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′ linked analogs of these, and those having inverted polarity wherein the adjacent pairs of nucleoside units are linked 3′-5′ to 5′-3′ or 2′-5′ to 5′-2′; see U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455, 233; 5,466,677; 5,476,925; 5,519,126; 5,536,821; 5,541,306; 5,550,111; 5,563, 253; 5,571,799; 5,587,361; and 5,625,050.

Morpholino-based oligomeric compounds are described in Dwaine A. Braasch and David R. Corey, Biochemistry, 2002, 41(14), 4503-4510); Genesis, volume 30, issue 3, 2001; Heasman, J., Dev. Biol., 2002, 243, 209-214; Nasevicius et al., Nat. Genet., 2000, 26, 216-220; Lacerra et al., Proc. Natl. Acad. Sci., 2000, 97, 9591-9596; and U.S. Pat. No. 5,034,506, issued Jul. 23, 1991. In some embodiments, the morpholino-based oligomeric compound is a phosphorodiamidate morpholino oligomer (PMO) (e.g., as described in Iverson, Curr. Opin. Mol. Ther., 3:235-238, 2001; and Wang et al., J. Gene Med., 12:354-364, 2010; the disclosures of which are incorporated herein by reference in their entireties).

Cyclohexenyl nucleic acid oligonucleotide mimetics are described in Wang et al., J. Am. Chem. Soc., 2000, 122, 8595-8602.

Modified oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These comprise those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; alkene containing backbones; sulfamate backbones; methyleneimino and methylenehydrazino backbones; sulfonate and sulfonamide backbones; amide backbones; and others having mixed N, O, S and CH2 component parts; see U.S. Pat. Nos. 5,034,506; 5,166,315; 5,185,444; 5,214,134; 5,216,141; 5,235,033; 5,264, 562; 5, 264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; and 5,677,439, each of which is herein incorporated by reference.

Modified oligonucleotides are also known that include oligonucleotides that are based on or constructed from arabinonucleotide or modified arabinonucleotide residues. Arabinonucleosides are stereoisomers of ribonucleosides, differing only in the configuration at the 2′-position of the sugar ring. In some embodiments, a 2′-arabino modification is 2′-F arabino. In some embodiments, the modified oligonucleotide is 2′-fluoro-D-arabinonucleic acid (FANA) (as described in, for example, Lon et al., Biochem., 41:3457-3467, 2002 and Min et al., Bioorg. Med. Chem. Lett., 12:2651-2654, 2002; the disclosures of which are incorporated herein by reference in their entireties). Similar modifications can also be made at other positions on the sugar, particularly the 3′ position of the sugar on a 3′ terminal nucleoside or in 2′-5′ linked oligonucleotides and the 5′ position of 5′ terminal nucleotide.

PCT Publication No. WO 99/67378 discloses arabinonucleic acids (ANA) oligomers and their analogues for improved sequence specific inhibition of gene expression via association to complementary messenger RNA.

Other preferred modifications include ethylene-bridged nucleic acids (ENAs) (e.g., International Patent Publication No. WO 2005/042777, Morita et al., Nucleic Acid Res., Suppl 1:241-242, 2001; Surono et al., Hum. Gene Ther., 15:749-757, 2004; Koizumi, Curr. Opin. Mol. Ther., 8:144-149, 2006 and Horie et al., Nucleic Acids Symp. Ser (Oxf), 49:171-172, 2005; the disclosures of which are incorporated herein by reference in their entireties). Preferred ENAs include, but are not limited to, 2′-0,4′-C-ethylene-bridged nucleic acids.

Examples of LNAs are described in WO/2008/043753 and include compounds of the following general formula.

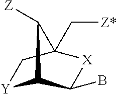

where X and Y are independently selected among the groups —O—,

—S—, —N(H)—, N(R)—, —CH₂— or —CH— (if part of a double bond),

—CH₂—O—, —CH₂—S—, —CH₂—N(H)—, —CH₂—N(R)—, —CH₂—CH₂— or —CH₂—CH— (if part of a double bond),

—CH═CH—, where R is selected from hydrogen and C₁₋₄-alkyl; Z and Z* are independently selected among an internucleoside linkage, a terminal group or a protecting group; B constitutes a natural or non-natural nucleotide base moiety; and the asymmetric groups may be found in either orientation.

Preferably, the LNA used in the oligonucleotides described herein comprises at least one LNA unit according any of the formulas

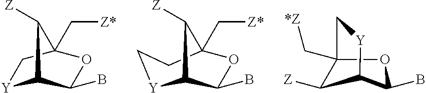

wherein Y is —O—, —S—, —NH—, or N(R^{H}); Z and Z* are independently selected among an internucleoside linkage, a terminal group or a protecting group; B constitutes a natural or non-natural nucleotide base moiety, and RH is selected from hydrogen and C₁₋₄-alkyl.

In some embodiments, the Locked Nucleic Acid (LNA) used in the oligonucleotides described herein comprises at least one Locked Nucleic Acid (LNA) unit according any of the formulas shown in Scheme 2 of PCT/DK2006/000512.

In some embodiments, the LNA used in the oligomer of the disclosure comprises internucleoside linkages selected from -0-P(O)₂—O—, —O—P(O,S)—O—, -0-P(S)₂—O—, —S—P(O)₂—O—, —S—P(O,S)—O—, —S—P(S)₂—O—, —O—P(O)₂—S—, —O—P(O,S)—S—, —S—P(O)₂—S—, —O—PO(R^{H})—O—, O—PO(OCH₃)—O—, —O—PO(NR^{H})—O—, —O—PO(OCH₂CH₂S—R)—O—, —O—PO(BH₃)—O—, —O—PO(NHR^{H})—O—, —O—P(O)₂—NR^{H}—, —NR^{H}—P(O)₂—O—, —NR^{H}—CO—O—, where R^{H} is selected from hydrogen and C₁₋₄-alkyl.

Specifically preferred LNA units are shown in scheme 2:

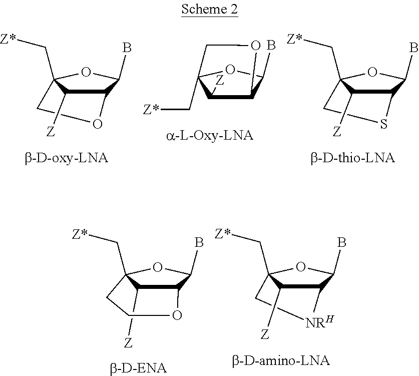

The term “thio-LNA” comprises a locked nucleotide in which at least one of X or Y in the general formula above is selected from S or —CH₂—S—. Thio-LNA can be in both beta-D and alpha-L-configuration.

The term “amino-LNA” comprises a locked nucleotide in which at least one of X or Y in the general formula above is selected from —N(H)—, N(R)—, CH₂—N(H)—, and —CH₂—N(R)— where R is selected from hydrogen and C₁₋₄-alkyl. Amino-LNA can be in both beta-D and alpha-L-configuration.

The term “oxy-LNA” comprises a locked nucleotide in which at least one of X or Y in the general formula above represents —O— or —CH₂—O—. Oxy-LNA can be in both beta-D and alpha-L-configuration.

The term “ena-LNA” comprises a locked nucleotide in which Y in the general formula above is —CH₂—O— (where the oxygen atom of —CH₂—O— is attached to the 2′-position relative to the base B).

LNAs are described in additional detail herein.

One or more substituted sugar moieties can also be included, e.g., one of the following at the 2′ position: OH, SH, SCH₃, F, OCN, OCH₃OCH₃, OCH₃O(CH₂)n CH₃, O(CH₂)n NH₂ or O(CH₂)n CH₃ where n is from 1 to about 10; C1 to C10 lower alkyl, alkoxyalkoxy, substituted lower alkyl, alkaryl or aralkyl; Cl; Br; CN; CF₃; OCF₃; O-, S-, or N-alkyl; O—, S—, or N-alkenyl; SOCH₃; SO₂CH₃; ONO₂; NO₂; N₃; NH2; heterocycloalkyl; heterocycloalkaryl; aminoalkylamino; polyalkylamino; substituted silyl; an RNA cleaving group; a reporter group; an intercalator; a group for improving the pharmacokinetic properties of an oligonucleotide; or a group for improving the pharmacodynamic properties of an oligonucleotide and other substituents having similar properties. A preferred modification includes 2′-methoxyethoxy [2′-O—CH₂CH₂OCH₃, also known as 2′-O-(2-methoxyethyl)] (Martin et al, Helv. Chim. Acta, 1995, 78, 486). Other preferred modifications include 2′-methoxy (2′-O—CH₃), 2′-propoxy (2′-OCH₂CH₂CH₃) and 2′-fluoro (2′-F). Similar modifications may also be made at other positions on the oligonucleotide, particularly the 3′ position of the sugar on the 3′ terminal nucleotide and the 5′ position of 5′ terminal nucleotide. Oligonucleotides may also have sugar mimetics such as cyclobutyls in place of the pentofuranosyl group.

Single stranded oligonucleotides can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl)adenine, 6-aminopurine, 2-aminopurine, 2-chloro-6-aminopurine and 2,6-diaminopurine or other diaminopurines. See, e.g., Kornberg, “DNA Replication,” W. H. Freeman & Co., San Francisco, 1980, pp 75-77; and Gebeyehu, G., et al. Nucl. Acids Res., 15:4513 (1987)). A “universal” base known in the art, e.g., inosine, can also be included. 5-Me-C substitutions have been shown to increase nucleic acid duplex stability by 0.6-1.2° C. (Sanghvi, in Crooke, and Lebleu, eds., Antisense Research and Applications, CRC Press, Boca Raton, 1993, pp. 276-278) and may be used as base substitutions.

It is not necessary for all positions in a given oligonucleotide to be uniformly modified, and in fact more than one of the modifications described herein may be incorporated in a single oligonucleotide or even at within a single nucleoside within an oligonucleotide.

In some embodiments, both a sugar and an internucleoside linkage, e.g., the backbone, of the nucleotide units are replaced with novel groups. The base units are maintained for hybridization with an appropriate nucleic acid target compound. One such oligomeric compound, an oligonucleotide mimetic that has been shown to have excellent hybridization properties, is referred to as a peptide nucleic acid (PNA). In PNA compounds, the sugar-backbone of an oligonucleotide is replaced with an amide containing backbone, for example, an aminoethylglycine backbone. The nucleobases are retained and are bound directly or indirectly to aza nitrogen atoms of the amide portion of the backbone. Representative United States patents that teach the preparation of PNA compounds include, but are not limited to, U.S. Pat. Nos. 5,539,082; 5,714,331; and 5,719,262, each of which is herein incorporated by reference. Further teaching of PNA compounds can be found in Nielsen et al, Science, 1991, 254, 1497-1500.

Single stranded oligonucleotides can also include one or more nucleobase (often referred to in the art simply as “base”) modifications or substitutions. As used herein, “unmodified” or “natural” nucleobases comprise the purine bases adenine (A) and guanine (G), and the pyrimidine bases thymine (T), cytosine (C) and uracil (U). Modified nucleobases comprise other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudo-uracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylquanine and 7-methyladenine, 8-azaguanine and 8-azaadenine, 7-deazaguanine and 7-deazaadenine and 3-deazaguanine and 3-deazaadenine.

Further, nucleobases comprise those disclosed in U.S. Pat. No. 3,687,808, those disclosed in “The Concise Encyclopedia of Polymer Science And Engineering”, pages 858-859, Kroschwitz, ed. John Wiley & Sons, 1990; those disclosed by Englisch et al., Angewandle Chemie, International Edition, 1991, 30, page 613, and those disclosed by Sanghvi, Chapter 15, Antisense Research and Applications,” pages 289-302, Crooke, and Lebleu, eds., CRC Press, 1993. Certain of these nucleobases are particularly useful for increasing the binding affinity of the oligomeric compounds of the disclosure. These include 5-substituted pyrimidines, 6-azapyrimidines and N-2, N-6 and 0-6 substituted purines, comprising 2-aminopropyladenine, 5-propynyluracil and 5-propynylcytosine. 5-methylcytosine substitutions have been shown to increase nucleic acid duplex stability by 0.6-1.2<0>C (Sanghvi, et al., eds, “Antisense Research and Applications,” CRC Press, Boca Raton, 1993, pp. 276-278) and are presently preferred base substitutions, even more particularly when combined with 2′-O-methoxyethyl sugar modifications. Modified nucleobases are described in U.S. Pat. No. 3,687,808, as well as U.S. Pat. Nos. 4,845,205; 5,130,302; 5,134,066; 5,175, 273; 5, 367,066; 5,432,272; 5,457,187; 5,459,255; 5,484,908; 5,502,177; 5,525,711; 5,552,540; 5,587,469; 5,596,091; 5,614,617; 5,750,692, and 5,681,941, each of which is herein incorporated by reference.

In some embodiments, the single stranded oligonucleotides are chemically linked to one or more moieties or conjugates that enhance the activity, cellular distribution, or cellular uptake of the oligonucleotide. For example, one or more single stranded oligonucleotides, of the same or different types, can be conjugated to each other; or single stranded oligonucleotides can be conjugated to targeting moieties with enhanced specificity for a cell type or tissue type. Such moieties include, but are not limited to, lipid moieties such as a cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S- tritylthiol (Manoharan et al, Ann. N. Y. Acad. Sci., 1992, 660, 306-309; Manoharan et al., Bioorg. Med. Chem. Let., 1993, 3, 2765-2770), a thiocholesterol (Oberhauser et al., Nucl. Acids Res., 1992, 20, 533-538), an aliphatic chain, e.g., dodecandiol or undecyl residues (Kabanov et al., FEBS Lett., 1990, 259, 327-330; Svinarchuk et al., Biochimie, 1993, 75, 49-54), a phospholipid, e.g., di-hexadecyl-rac-glycerol or triethylammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Mancharan et al., Nucleosides & Nucleotides, 1995, 14, 969-973), or adamantane acetic acid (Manoharan et al., Tetrahedron Lett., 1995, 36, 3651-3654), a palmityl moiety (Mishra et al., Biochim. Biophys. Acta, 1995, 1264, 229-237), or an octadecylamine or hexylamino-carbonyl-t oxycholesterol moiety (Crooke et al., J. Pharmacol. Exp. Ther., 1996, 277, 923-937). See also U.S. Pat. Nos. 4,828,979; 4,948,882; 5,218,105; 5,525,465; 5,541,313; 5,545,730; 5,552, 538; 5,578,717, 5,580,731; 5,580,731; 5,591,584; 5,109,124; 5,118,802; 5,138,045; 5,414,077; 5,486, 603; 5,512,439; 5,578,718; 5,608,046; 4,587,044; 4,605,735; 4,667,025; 4,762, 779; 4,789,737; 4,824,941; 4,835,263; 4,876,335; 4,904,582; 4,958,013; 5,082, 830; 5,112,963; 5,214,136; 5,082,830; 5,112,963; 5,214,136; 5, 245,022; 5,254,469; 5,258,506; 5,262,536; 5,272,250; 5,292,873; 5,317,098; 5,371,241, 5,391, 723; 5,416,203, 5,451,463; 5,510,475; 5,512,667; 5,514,785; 5, 565,552; 5,567,810; 5,574,142; 5,585,481; 5,587,371; 5,595,726; 5,597,696; 5,599,923; 5,599, 928, and 5,688,941, each of which is herein incorporated by reference.

These moieties or conjugates can include conjugate groups covalently bound to functional groups such as primary or secondary hydroxyl groups. Conjugate groups of the disclosure include intercalators, reporter molecules, polyamines, polyamides, polyethylene glycols, polyethers, groups that enhance the pharmacodynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this disclosure, include groups that improve uptake, enhance resistance to degradation, and/or strengthen sequence-specific hybridization with the target nucleic acid. Groups that enhance the pharmacokinetic properties, in the context of this disclosure, include groups that improve uptake, distribution, metabolism, or excretion of the compounds of the present disclosure. Representative conjugate groups are disclosed in International Patent Application No. PCT/US92/09196, filed Oct. 23, 1992, and U.S. Pat. No. 6,287,860, which are incorporated herein by reference. Conjugate moieties include, but are not limited to, lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethylammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxy cholesterol moiety. See, e.g., U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928, and 5,688,941.

In some embodiments, single stranded oligonucleotide modification includes modification of the 5′ or 3′ end of the oligonucleotide. In some embodiments, the 3′ end of the oligonucleotide comprises a hydroxyl group or a thiophosphate. It should be appreciated that additional molecules (e.g., a biotin moiety or a fluorophor) can be conjugated to the 5′ or 3′ end of the single stranded oligonucleotide. In some embodiments, the single stranded oligonucleotide comprises a biotin moiety conjugated to the 5′ nucleotide.

In some embodiments, the single stranded oligonucleotide comprises locked nucleic acids (LNA), ENA modified nucleotides, 2′-O-methyl nucleotides, or 2′-fluoro-deoxyribonucleotides. In some embodiments, the single stranded oligonucleotide comprises alternating deoxyribonucleotides and 2′-fluoro-deoxyribonucleotides. In some embodiments, the single stranded oligonucleotide comprises alternating deoxyribonucleotides and 2′-O-methyl nucleotides. In some embodiments, the single stranded oligonucleotide comprises alternating deoxyribonucleotides and ENA modified nucleotides. In some embodiments, the single stranded oligonucleotide comprises alternating deoxyribonucleotides and locked nucleic acid nucleotides. In some embodiments, the single stranded oligonucleotide comprises alternating locked nucleic acid nucleotides and 2′-O-methyl nucleotides.

In some embodiments, the 5′ nucleotide of the oligonucleotide is a deoxyribonucleotide. In some embodiments, the 5′ nucleotide of the oligonucleotide is a locked nucleic acid nucleotide. In some embodiments, the nucleotides of the oligonucleotide comprise deoxyribonucleotides flanked by at least one locked nucleic acid nucleotide on each of the 5′ and 3′ ends of the deoxyribonucleotides. In some embodiments, the nucleotide at the 3′ position of the oligonucleotide has a 3′ hydroxyl group or a 3′ thiophosphate.

In some embodiments, the single stranded oligonucleotide comprises phosphorothioate internucleotide linkages. In some embodiments, the single stranded oligonucleotide comprises phosphorothioate internucleotide linkages between at least two nucleotides. In some embodiments, the single stranded oligonucleotide comprises phosphorothioate internucleotide linkages between all nucleotides.

In some embodiments, oligonucleotides include only one type of internucleoside linkage (e.g., oligonucleotides may be fully phosphorothioated). However, in some embodiments, oligonucleotides include a mix of different internucleoside linkages (e.g., a mix of phosphorothioate and phosphodiester linkages). For example, in some embodiments, oligonucleotides may include 50% phosphorothioate linkages and 50% phosphodiester linkages. In some embodiments, oligonucleotides provided herein may have a central stretch of 2, 3, 4, 5, 6, 7, or more nucleotide residues linked by a first linkage type, and flanking nucleotide residues that are linked by a second linkage type. In some embodiments, oligonucleotides provided herein may have a central stretch of 2, 3, 4, 5, 6, 7, or more nucleotide residues linked by phosphodiester linkages, and flanking nucleotide residues that are linked by phosphorothioates. In some embodiments, flanking nucleotide residues are independently 2, 3, 4, 5, 6, 7 or more nucleotide residues in length.

It should be appreciated that the single stranded oligonucleotide can have any combination of modifications as described herein.

The oligonucleotide may comprise a nucleotide sequence having one or more of the following modification patterns.

(a) (X)Xxxxxx, (X)xXxxxx, (X)xxXxxx, (X)xxxXxx, (X)xxxxXx and (X)xxxxxX,

(b) (X)XXxxxx, (X)XxXxxx, (X)XxxXxx, (X)XxxxXx, (X)XxxxxX, (X)xXXxxx, (X)xXxXxx, (X)xXxxXx, (X)xXxxxX, (X)xxXXxx, (X)xxXxXx, (X)xxXxxX, (X)xxxXXx, (X)xxxXxX and (X)xxxxXX,

(c) (X)XXXxxx, (X)xXXXxx, (X)xxXXXx, (X)xxxXXX, (X)XXxXxx, (X)XXxxXx, (X)XXxxxX, (X)xXXxXx, (X)xXXxxX, (X)xxXXxX, (X)XxXXxx, (X)XxxXXx (X)XxxxXX, (X)xXxXXx, (X)xXxxXX, (X)xxXxXX, (X)xXxXxX and (X)XxXxXx,

(d) (X)xxXXX, (X)xXxXXX, (X)xXXxXX, (X)xXXXxX, (X)xXXXXx, (X)XxxXXXX, (X)XxXxXX, (X)XxXXxX, (X)XxXXx, (X)XXxxXX, (X)XXxXxX, (X)XXxXXx, (X)XXXxxX, (X)XXXxXx, and (X)XXXXxx,

(e) (X)xXXXXX, (X)XxXXXX, (X)XXxXXX, (X)XXXxXX, (X)XXXXxX and (X)XXXXXx, and

(f) XXXXXX, XxXXXXX, XXxXXXX, XXXxXXX, XXXXxXX, XXXXXxX and XXXXXXx, in which “X” denotes a nucleotide analogue, (X) denotes an optional nucleotide analogue, and “x” denotes a DNA or RNA nucleotide unit. Each of the above listed patterns may appear one or more times within an oligonucleotide, alone or in combination with any of the other disclosed modification patterns.

Oligonucleotides described herein may be modified, e.g., comprise a modified sugar moiety, a modified internucleoside linkage, a modified nucleotide and/or combinations thereof. In addition, the oligonucleotides can exhibit one or more of the following properties: do not induce substantial cleavage or degradation of the target RNA; do not cause substantially complete cleavage or degradation of the target RNA; do not activate the RNase H pathway; do not activate RISC; do not recruit any Argonaute family protein; are not cleaved by Dicer; do not mediate alternative splicing; are not immune stimulatory; are nuclease resistant; have improved cell uptake compared to unmodified oligonucleotides; are not toxic to cells or mammals; may have improved endosomal exit; do interfere with interaction of lncRNA with PRC2, preferably the Ezh2 subunit but optionally the Suz12, Eed, RbAp46/48 subunits or accessory factors such as Jarid2; do decrease histone H3 lysine27 methylation and/or do upregulate gene expression.

### Methods for Modulating Gene Expression

In some embodiments, methods are provided for increasing expression of SMN protein in a cell. The methods, in some embodiments, involve delivering to the cell a first single stranded oligonucleotide complementary with a PRC2-associated region of SMN and a second single stranded oligonucleotide complementary with a splice control sequence of a precursor mRNA of SMN, in amounts sufficient to increase expression of a mature mRNA of SMN that comprises (or includes) exon 7 in the cell. The first and second single stranded oligonucleotides may be delivered together or separately. The first and second single stranded oligonucleotides may be linked together, or unlinked.

In some embodiments, methods are provided for treating spinal muscular atrophy or other condition (e.g., ALS) in a subject. The methods, in some embodiments, involve administering to a subject a first single stranded oligonucleotide complementary with a PRC2-associated region and a second single stranded oligonucleotide complementary with a splice control sequence of a precursor mRNA of SMN, in amounts sufficient to increase expression of full length SMN protein in the subject to levels sufficient to improve one or more conditions associated with SMA. The first and second single stranded oligonucleotides may be administered together or separately. The first and second single stranded oligonucleotides may be linked together, or unlinked, e.g., separate. The first single stranded oligonucleotide may be administered within 1 hour, 2 hours, 3 hours, 4 hours, 8 hours, 12 hours, 24 hours, 48 hours, or more of administration of the second single stranded oligonucleotide. The first single stranded oligonucleotide may be administered before or after the second single stranded oligonucleotide. The oligonucleotides may be administered once or on multiple occasions depending on the needs of the subject and/or judgment of the treating physician. In some cases, the oligonucleotides may be administered in cycles. The administration cycles may vary; for example, the administration cycle may be 2^{nd} oligonucleotide (oligo)—1^{st} oligo—2^{nd} oligo—1^{st} oligo and so on; or 1^{st} oligo—2^{nd} oligo—1^{st} oligo—2^{nd} oligo, and so on; or 1^{st} oligo—2^{nd} oligo—2^{nd} oligo—1^{st} oligo—1^{st} oligo—2^{nd} oligo—2^{nd} oligo—1^{st} oligo, and so on. The skilled artisan will be capable of selecting administration cycles and intervals between each administration that are appropriate for treating a particular subject.

In certain aspects, the disclosure relates to methods for modulating gene expression in a cell (e.g., a cell for which SMN levels are reduced) for research purposes (e.g., to study the function of the gene in the cell). In another aspect, the disclosure relates to methods for modulating gene expression in a cell (e.g., a cell for which SMN levels are reduced) for gene or epigenetic therapy. The cells can be in vitro, ex vivo, or in vivo (e.g., in a subject who has a disease resulting from reduced expression or activity of SMN). In some embodiments, methods for modulating gene expression in a cell comprise delivering a single stranded oligonucleotide as described herein. In some embodiments, delivery of the single stranded oligonucleotide to the cell results in a level of expression of gene that is at least 5%, 10%, 20%, 30%, 40%, 50%, 60%, 70%, 80%, 90%, 100%, 200%, or more greater than a level of expression of gene in a control cell to which the single stranded oligonucleotide has not been delivered. In certain embodiments, delivery of the single stranded oligonucleotide to the cell results in a level of expression of gene that is at least 50% greater than a level of expression of gene in a control cell to which the single stranded oligonucleotide has not been delivered.

In other aspects of the disclosure, methods comprise administering to a subject (e.g., a human) a composition comprising a single stranded oligonucleotide as described herein to increase protein levels in the subject. In some embodiments, the increase in protein levels is at least 5%, 10%, 20%, 30%, 40%, 50%, 60%, 70%, 80%, 90%, 100%, 200%, or more, higher than the amount of a protein in the subject before administering.

As another example, to increase expression of SMN in a cell, the methods include introducing into the cell a single stranded oligonucleotide that is sufficiently complementary to a PRC2-associated region (e.g., of a long non-coding RNA) that maps to a genomic position encompassing or in proximity to the SMN gene.

In other aspects of the disclosure provides methods of treating a condition (e.g., Spinal Muscular Atrophy) associated with decreased levels of expression of SMN in a subject, the method comprising administering a single stranded oligonucleotide as described herein.

A subject can include a non-human mammal, e.g., mouse, rat, guinea pig, rabbit, cat, dog, goat, cow, or horse. In preferred embodiments, a subject is a human. Single stranded oligonucleotides have been employed as therapeutic moieties in the treatment of disease states in animals, including humans. Single stranded oligonucleotides can be useful therapeutic modalities that can be configured to be useful in treatment regimes for the treatment of cells, tissues and animals, especially humans.

For therapeutics, an animal, preferably a human, suspected of having Spinal muscular atrophy is treated by administering single stranded oligonucleotide in accordance with this disclosure. For example, in one non-limiting embodiment, the methods comprise the step of administering to the animal in need of treatment, a therapeutically effective amount of a single stranded oligonucleotide as described herein.

### Formulation, Delivery, And Dosing

The oligonucleotides described herein can be formulated for administration to a subject for treating a condition (e.g., Spinal muscular atrophy) associated with decreased levels of SMN protein. It should be understood that the formulations, compositions and methods can be practiced with any of the oligonucleotides disclosed herein. In some embodiments, formulations are provided that comprise a first single stranded oligonucleotide complementary with a PRC2-associated region of a gene and a second single stranded oligonucleotide complementary to a splice control sequence of a precursor mRNA of the gene. In some embodiments, formulations are provided that comprise a first single stranded oligonucleotide complementary with a PRC2-associated region of a gene that is linked via a linker with a second single stranded oligonucleotide complementary to a splice control sequence of a precursor mRNA of the gene. Thus, it should be appreciated that in some embodiments, a first single stranded oligonucleotide complementary with a PRC2-associated region of a gene is linked with a second single stranded oligonucleotide complementary to a splice control sequence of a precursor mRNA of the gene, and in other embodiments, the single stranded oligonucleotides are not linked. Single stranded oligonucleotides that are not linked may be administered to a subject or delivered to a cell simultaneously (e.g., within the same composition) or separately.

The formulations may conveniently be presented in unit dosage form and may be prepared by any methods well known in the art of pharmacy. The amount of active ingredient (e.g., an oligonucleotide or compound of the disclosure) which can be combined with a carrier material to produce a single dosage form will vary depending upon the host being treated and the particular mode of administration, e.g., intradermal or inhalation. The amount of active ingredient which can be combined with a carrier material to produce a single dosage form will generally be that amount of the compound which produces a therapeutic effect, e.g., tumor regression.

Pharmaceutical formulations of this disclosure can be prepared according to any method known to the art for the manufacture of pharmaceuticals. Such formulations can contain sweetening agents, flavoring agents, coloring agents, and preserving agents. A formulation can be admixtured with nontoxic pharmaceutically acceptable excipients which are suitable for manufacture. Formulations may comprise one or more diluents, emulsifiers, preservatives, buffers, excipients, etc. and may be provided in such forms as liquids, powders, emulsions, lyophilized powders, sprays, creams, lotions, controlled release formulations, tablets, pills, gels, on patches, in implants, etc.

A formulated single stranded oligonucleotide composition can assume a variety of states. In some examples, the composition is at least partially crystalline, uniformly crystalline, and/or anhydrous (e.g., less than 80, 50, 30, 20, or 10% water). In another example, the single stranded oligonucleotide is in an aqueous phase, e.g., in a solution that includes water. The aqueous phase or the crystalline compositions can, e.g., be incorporated into a delivery vehicle, e.g., a liposome (particularly for the aqueous phase) or a particle (e.g., a microparticle as can be appropriate for a crystalline composition). Generally, the single stranded oligonucleotide composition is formulated in a manner that is compatible with the intended method of administration.

In some embodiments, the composition is prepared by at least one of the following methods: spray drying, lyophilization, vacuum drying, evaporation, fluid bed drying, or a combination of these techniques; or sonication with a lipid, freeze-drying, condensation, and other self-assembly.

A single stranded oligonucleotide preparation can be formulated or administered (together or separately) in combination with another agent, e.g., another therapeutic agent or an agent that stabilizes a single stranded oligonucleotide, e.g., a protein that complexes with single stranded oligonucleotide. Still other agents include chelators, e.g., EDTA (e.g., to remove divalent cations such as Mg²⁺), salts, RNase inhibitors (e.g., a broad specificity RNase inhibitor such as RNAsin) and so forth. In some embodiments, the other agent used in combination with the single stranded oligonucleotide is an agent that also regulates SMN expression. In some embodiments, the other agent is a growth hormone, a histone deacetylase inhibitor, a hydroxycarbamide (hydroxyurea), a natural polyphenol compound (e.g., resveratrol, curcumin), prolactin, or salbutamol. Examples of histone deacetylase inhibitors that may be used include aliphatic compounds (e.g., butyrates (e.g., sodium butyrate and sodium phenylbutyrate) and valproic acid), benzamides (e.g., M344), and hydroxamic acids (e.g., CBHA, SBHA, Entinostat (MS-275)) Panobinostat (LBH-589), Trichostatin A, Vorinostat (SAHA)),

In one embodiment, the single stranded oligonucleotide preparation includes another single stranded oligonucleotide, e.g., a second single stranded oligonucleotide that modulates expression and/or mRNA processing of a second gene or a second single stranded oligonucleotide that modulates expression of the first gene. Still other preparation can include at least 3, 5, ten, twenty, fifty, or a hundred or more different single stranded oligonucleotide species. Such single stranded oligonucleotides can mediate gene expression with respect to a similar number of different genes. In one embodiment, the single stranded oligonucleotide preparation includes at least a second therapeutic agent (e.g., an agent other than an oligonucleotide).

### Route of Delivery

A composition that includes a single stranded oligonucleotide can be delivered to a subject by a variety of routes. Exemplary routes include: intrathecal, intracerebral, intramuscular, intravenous, intradermal, topical, rectal, parenteral, anal, intravaginal, intranasal, pulmonary, ocular, etc. The term “therapeutically effective amount” is the amount of oligonucleotide present in the composition that is needed to provide the desired level of SMN expression in the subject to be treated to give the anticipated physiological response. The term “physiologically effective amount” is that amount delivered to a subject to give the desired palliative or curative effect. The term “pharmaceutically acceptable carrier” means that the carrier can be administered to a subject with no significant adverse toxicological effects to the subject.

The single stranded oligonucleotide molecules of the disclosure can be incorporated into pharmaceutical compositions suitable for administration. Such compositions typically include one or more species of single stranded oligonucleotide and a pharmaceutically acceptable carrier. As used herein the language “pharmaceutically acceptable carrier” is intended to include any and all solvents, dispersion media, coatings, antibacterial and antifungal agents, isotonic and absorption delaying agents, and the like, compatible with pharmaceutical administration. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

The pharmaceutical compositions of the present disclosure may be administered in a number of ways depending upon whether local or systemic treatment is desired and upon the area to be treated. Administration may be topical (including ophthalmic, vaginal, rectal, intranasal, transdermal), oral, or parenteral. In one embodiment, administration is parenteral, e.g., intramuscular, intravenous (e.g., as a bolus or as a diffusible infusion), intradermal, intraperitoneal, intrathecal, intraventricular, intracranial, subcutaneous, transmucosal, buccal, sublingual, endoscopic, rectal, oral, vaginal, topical, pulmonary, intranasal, urethral, or ocular. Administration can be provided by the subject or by another person, e.g., a health care provider.

The route and site of administration may be chosen to enhance targeting. For example, to target muscle cells, intramuscular injection into the muscles of interest would be a logical choice.

Topical administration refers to the delivery to a subject by contacting the formulation directly to a surface of the subject. The most common form of topical delivery is to the skin, but a composition disclosed herein can also be directly applied to other surfaces of the body, e.g., to the eye, a mucous membrane, to surfaces of a body cavity or to an internal surface. Formulations for topical administration may include transdermal patches, ointments, lotions, creams, gels, drops, suppositories, sprays, liquids, and powders. Conventional pharmaceutical carriers, aqueous, powder or oily bases, thickeners, and the like may be necessary or desirable.

Compositions for oral administration include powders or granules, suspensions or solutions in water, syrups, slurries, emulsions, elixirs or non-aqueous media, tablets, capsules, lozenges, or troches. In the case of tablets, carriers that can be used include lactose, sodium citrate and salts of phosphoric acid. Various disintegrants such as starch, and lubricating agents such as magnesium stearate, sodium lauryl sulfate and talc, are commonly used in tablets. For oral administration in capsule form, useful diluents are lactose and high molecular weight polyethylene glycols. When aqueous suspensions are required for oral use, the nucleic acid compositions can be combined with emulsifying and suspending agents. If desired, certain sweetening and/or flavoring agents can be added.

Parenteral administration includes intravenous drip, subcutaneous, intraperitoneal or intramuscular injection, intrathecal or intraventricular administration. In some embodiments, parental administration involves administration directly to the site of disease (e.g., injection into a tumor).

Formulations for parenteral administration may include sterile aqueous solutions which may also contain buffers, diluents, and other suitable additives. Intraventricular injection may be facilitated by an intraventricular catheter, for example, attached to a reservoir. For intravenous use, the total concentration of solutes should be controlled to render the preparation isotonic.

Pulmonary delivery compositions can be delivered by inhalation by the patient of a dispersion so that the composition, preferably single stranded oligonucleotides, within the dispersion can reach the lung where it can be readily absorbed through the alveolar region directly into blood circulation. Pulmonary delivery can be effective both for systemic delivery and for localized delivery to treat diseases of the lungs.

The types of pharmaceutical excipients that are useful as carriers include stabilizers such as human serum albumin (HSA), bulking agents such as carbohydrates, amino acids and polypeptides; pH adjusters or buffers; salts such as sodium chloride; and the like. These carriers may be in a crystalline or amorphous form or may be a mixture of the two.

Suitable pH adjusters or buffers include organic salts prepared from organic acids and bases, such as sodium citrate, sodium ascorbate, and the like; sodium citrate is preferred. Pulmonary administration of a micellar single stranded oligonucleotide formulation may be achieved through metered dose spray devices with propellants such as tetrafluoroethane, heptafluoroethane, dimethylfluoropropane, tetrafluoropropane, butane, isobutane, dimethyl ether, and other non-CFC and CFC propellants.

Exemplary delivery devices include devices which are introduced into the vasculature, e.g., devices inserted into the lumen of a vascular tissue, or which devices themselves form a part of the vasculature, including stents, catheters, heart valves, and other vascular devices. These devices, e.g., catheters or stents, can be placed in the vasculature of the lung, heart, or leg.

Other devices include non-vascular devices, e.g., devices implanted in the peritoneum, or in organ or glandular tissue, e.g., artificial organs. The device can release a therapeutic substance in addition to a single stranded oligonucleotide, e.g., a device can release insulin.

In some embodiments, unit doses or measured doses of a composition that includes single stranded oligonucleotides are dispensed by an implanted device. The device can include a sensor that monitors a parameter within a subject. For example, the device can include pump, e.g., and, optionally, associated electronics.

Tissue, e.g., cells or organs can be treated with a single stranded oligonucleotide, ex vivo and then administered or implanted in a subject. The tissue can be autologous, allogeneic, or xenogeneic tissue. For example, tissue can be treated to reduce graft v. host disease. In other embodiments, the tissue is allogeneic and the tissue is treated to treat a disorder characterized by unwanted gene expression in that tissue. For example, tissue, e.g., hematopoietic cells, e.g., bone marrow hematopoietic cells, can be treated to inhibit unwanted cell proliferation. Introduction of treated tissue, whether autologous or transplant, can be combined with other therapies. In some implementations, the single stranded oligonucleotide treated cells are insulated from other cells, e.g., by a semi-permeable porous barrier that prevents the cells from leaving the implant, but enables molecules from the body to reach the cells and molecules produced by the cells to enter the body. In some embodiments, the porous barrier is formed from alginate.

### Dosage

In some aspects, the disclosure features methods of administering single stranded oligonucleotides (e.g., as a compound or as a component of a composition) to a subject (e.g., a human subject). For example, transcriptional oligonucleotides can be effective in vivo when combined with either oligonucleotides or small molecules that promote correct splicing of SMN2 transcripts. A variety of doses, routes of administration, and dosing regiments can be employed.

In order to access the central nervous system (CNS), the two agents may be administered in mouse models of SMA by either intracerebroventricular (ICV) or intrathecal (IT) injection. In human clinical use, IT injection is a useful route of administration into the CNS. The IT injection can be a bolus injection or longer term infusion. In mouse models of SMA, systemic exposure to the SMN2 upregulating agents has beneficial effects due to involvement of SMN protein in peripheral tissues. For systemic exposure administration of oligonucleotides may be achieved by subcutaneous (SC) injection, although intravenous (IV) and intraperitoneal (IP) routes also may be used. To achieve both CNS and peripheral tissue exposure in human patients, both IT and SC injections may be used. Due to the long half-life of oligonucleotides in the brain and spinal cord, IT injections may be in a range of once every 3 months to once every 6 months; however, in some embodiments, multiple injections at closer intervals may be used at the start of treatment as a “loading” regimen. A variety of dose schedules may be used for SC injection, with once monthly injection being an example regimen.

In some embodiments, the methods involve administering an agent (e.g., a single stranded oligonucleotide) in a unit dose to a subject. In one embodiment, the unit dose is between about 10 mg and 25 mg per kg of bodyweight. In one embodiment, the unit dose is between about 1 mg and 100 mg per kg of bodyweight. In one embodiment, the unit dose is between about 0.1 mg and 500 mg per kg of bodyweight. In some embodiments, the unit dose is more than 0.001, 0.005, 0.01, 0.05, 0.1, 0.5, 1, 2, 5, 10, 25, 50, or 100 mg per kg of bodyweight.

The defined amount can be an amount effective to treat or prevent a disease or disorder, e.g., a disease or disorder associated with the SMN. The unit dose, for example, can be administered by injection (e.g., intravenous or intramuscular), an inhaled dose, or a topical application.

In some embodiments, the unit dose is administered daily. In some embodiments, less frequently than once a day, e.g., less than every 2, 4, 8 or 30 days. In another embodiment, the unit dose is not administered with a frequency (e.g., not a regular frequency). For example, the unit dose may be administered a single time. In some embodiments, the unit dose is administered more than once a day, e.g., once an hour, two hours, four hours, eight hours, twelve hours, etc.

In one embodiment, a subject is administered an initial dose and one or more maintenance doses of a single stranded oligonucleotide. The maintenance dose or doses are generally lower than the initial dose, e.g., one-half less of the initial dose. A maintenance regimen can include treating the subject with a dose or doses ranging from 0.0001 to 100 mg/kg of body weight per day, e.g., 100, 10, 1, 0.1, 0.01, 0.001, or 0.0001 mg per kg of bodyweight per day. The maintenance doses may be administered no more than once every 1, 5, 10, or 30 days. Further, the treatment regimen may last for a period of time which will vary depending upon the nature of the particular disease, its severity and the overall condition of the patient. In some embodiments, the dosage may be delivered no more than once per day, e.g., no more than once per 24, 36, 48, or more hours, e.g., no more than once for every 5 or 8 days. Following treatment, the patient can be monitored for changes in his condition and for alleviation of the symptoms of the disease state. The dosage of the oligonucleotide may either be increased in the event the patient does not respond significantly to current dosage levels, or the dose may be decreased if an alleviation of the symptoms of the disease state is observed, if the disease state has been ablated, or if undesired side-effects are observed.

The effective dose can be administered in a single dose or in two or more doses, as desired or considered appropriate under the specific circumstances.

The transcriptional and splice correcting agents may be administered together, e.g., simultaneously. Alternatively, the dosing of the two different agents could be staggered such that one may be administered prior to the other. In some embodiments, splice correcting agents tend to act more rapidly than the transcriptional oligonucleotides. In some embodiments, splice correcting agents promote the splicing of the SMN2 mRNA in a co-transcriptional manner. Since some SMN2 RNA will be synthesized in cells prior to treatment, splice correcting agents can rapidly act to promote the inclusion of exon 7 during the spicing process. In contrast, it is believed that the transcriptional oligonucleotides must induce the remodeling of the SMN2 gene chromatin in order to elevate SMN2 transcription. This process appears to consist of blocking the application of the repressive chromatin mark that is mediated by PRC2. Histone demethylases may be required to remove the H3K27me3 repressive histone modification that is already present on chromatin. These processes lead to the upregulation of transcription of the SMN2 gene. Since this process is slower than splice-correction, one alternative approach to dosing both agents simultaneously is to dose with the transcriptional oligonucleotide first to increase the amounts of SMN2 RNA followed by dosing with the splice correcting agent to then correct the splicing of the upregulated SMN2 RNA.

If simultaneous administration is desired, the two agents either could be mixed together or actually covalently linked in one chemical composition. For instance, two oligonucleotides could be linked in a Multi-Target Oligonucleotide (MTO). In this composition, the two separate oligonucleotide sequences are joined together in one oligonucleotide and are separated by a cleavable linker. This linker could be a nucleotide or non-nucleotide linker. In one embodiment, the two oligonucleotide sequences are separated by 2, 3 or 4 DNA nucleotides, typically poly dA or dT. The SMN2 upregulating sequences are heavily modified for increased stability. The MTO is stable in blood and tissues. Once taken up into cells, the linker in the MTO is cleaved within endosomes in the cells, thus releasing the two separate SMN2 upregulating oligonucleotides to act via their distinct mechanisms of action and target sites.

Accordingly, in some embodiments, a pharmaceutical composition includes a plurality of single stranded oligonucleotide species. In some embodiments, the pharmaceutical composition comprises a first single stranded oligonucleotide complementary with a PRC2-associated region of a gene (e.g., SMN), and a second single stranded oligonucleotide complementary to a splice control sequence of a precursor mRNA of a gene (e.g., SMN). In some embodiments, the pharmaceutical composition includes a compound comprising the general formula A-B-C, in which A is a single stranded oligonucleotide complementary with a PRC2-associated region of a gene, B is a linker, and C is a single stranded oligonucleotide complementary to a splice control sequence of a precursor mRNA of the gene.

Following successful treatment, it may be desirable to have the patient undergo maintenance therapy to prevent the recurrence of the disease state, wherein the compound of the disclosure is administered in maintenance doses, ranging from 0.0001 mg to 100 mg per kg of body weight.

The concentration of the single stranded oligonucleotide composition is an amount sufficient to be effective in treating or preventing a disorder or to regulate a physiological condition in humans. The concentration or amount of single stranded oligonucleotide administered will depend on the parameters determined for the agent and the method of administration, e.g., intramuscular administration.

Certain factors may influence the dosage required to effectively treat a subject, including but not limited to the severity of the disease or disorder, previous treatments, the general health and/or age of the subject, and other diseases present. Moreover, treatment of a subject with a therapeutically effective amount of a single stranded oligonucleotide can include a single treatment or, preferably, can include a series of treatments. It will also be appreciated that the effective dosage of a single stranded oligonucleotide used for treatment may increase or decrease over the course of a particular treatment. For example, the subject can be monitored after administering a single stranded oligonucleotide composition. Based on information from the monitoring, an additional amount of the single stranded oligonucleotide composition can be administered.

Dosing is dependent on severity and responsiveness of the disease condition to be treated, with the course of treatment lasting from several days to several months, or until a cure is effected or a diminution of disease state is achieved. Optimal dosing schedules can be calculated from measurements of SMN expression levels in the body of the patient. Persons of ordinary skill can easily determine optimum dosages, dosing methodologies and repetition rates. Optimum dosages may vary depending on the relative potency of individual compounds, and can generally be estimated based on EC50s found to be effective in in vitro and in vivo animal models. In some embodiments, the animal models include transgenic animals that express a human SMN. In some embodiments, the composition for testing includes a single stranded oligonucleotide that is complementary, at least in an internal region, to a sequence that is conserved between SMN in the animal model and SMN in a human.

### Kits

In certain aspects of the disclosure, kits are provided, comprising a container housing a composition comprising a single stranded oligonucleotide. In some embodiments, the kits comprise a container housing a single stranded oligonucleotide complementary with a PRC2-associated region of a gene; and a second container housing a single stranded oligonucleotide complementary to a splice control sequence of a precursor mRNA of the gene. In some embodiments, the kits comprise a container housing a single stranded oligonucleotide complementary with of a PRC2-associated region and a splice correcting agent (e.g., a single stranded oligonucleotide complementary to a splice control sequence of a precursor mRNA of SMN). In some embodiments, the composition is a pharmaceutical composition comprising a single stranded oligonucleotide and a pharmaceutically acceptable carrier. In some embodiments, the individual components of the pharmaceutical composition may be provided in one container. Alternatively, it may be desirable to provide the components of the pharmaceutical composition separately in two or more containers, e.g., one container for single stranded oligonucleotides, and at least another for a carrier compound. The kit may be packaged in a number of different configurations such as one or more containers in a single box. The different components can be combined, e.g., according to instructions provided with the kit. The components can be combined according to a method described herein, e.g., to prepare and administer a pharmaceutical composition. The kit can also include a delivery device.

The present disclosure is further illustrated by the following Examples, which in no way should be construed as further limiting. The entire contents of all of the references (including literature references, issued patents, published patent applications, and co-pending patent applications) cited throughout this application are hereby expressly incorporated by reference.

### EXAMPLES

### Example 1. Therapeutic Agents that Promote the Proper Splicing of SMN2 Transcripts by Increasing Exon 7 Inclusion

A PRC2-binding lncRNA that is antisense to the SMN2 gene (“SMN-AS”) and binds PRC2 has been identified. Data indicate that SMN-AS recruits PRC2 to the SMN2 gene and represses SMN2 transcription by an epigenetic mechanism. This repressive mechanism is the trimethylation of histone 3 at lysine 27 (the H3K27me3 repressive mark). The specific oligonucleotides sterically block the association of SMN-AS with PRC2, thus inhibiting the application of the H3K27me3 repressive chromatin mark. Chromatin immunoprecipitation (ChIP) experiments have been performed which demonstrate that oligonucleotides targeting this SMN-AS decreases PRC2 association with the SMN gene, decreases the presence of H3K27me3 along the SMN gene, and activates SMN2 transcription as seen by an increase in the H3K36me3 transcriptional activating mark and an increase in the presence of transcribing RNA polymerase II along the length of the SMN gene. This leads to a significant increase in SMN2 mRNA and protein in human SMA patient cells.

Oligonucleotides have been identified that increase the amount of full-length SMN2 mRNA without increasing the de17 form of SMN2 mRNA. In some cases, the de17 form actually decreases, although the decrease is not as significant as that induced by splice-correcting oligonucleotides or small molecules that affect splicing. The basis for the effect of these oligonucleotides on SMN2 splicing may be due to either (a) the increased SMN protein inducing its own splice correction (since SMN protein plays a role in splicing, there may be a “feed-forward” mechanism in which the transcriptional oligonucleotides increase SMN protein level which then serves to improve SMN2 splicing efficiency), or (b) the SMN antisense transcript and PRC2 also could affect SMN2 splicing as well as regulating transcription.

Mechanism of action studies show that oligonucleotides targeting PRC2-associated region of SMN-AS act by increasing SMN2 transcription. Such oligonucleotides are referred to in this example as transcriptional oligonucleotides because they affect SMN transcription as compared with splicing. In contrast, splice-correcting oligonucleotides and small molecules act by promoting the inclusion of exon 7 during splicing of SMN2 RNA, without necessarily affecting transcription.

In some embodiments, two approaches (transcriptional and splice correcting) have been combined to produce an even greater increase in SMN levels or to increase SMN while dosing with lower amounts of these agents. In some embodiments, the combinations have synergistic effects. In some embodiments, the transcriptional oligonucleotides increase the amount of SMN2 RNA and this activity creates more SMN2 RNA substrate whose splicing can be corrected by splice-correcting oligonucleotides and/or small molecules that affect splicing. In some embodiments, since these two approaches act at different points in SMN2 expression, they are not redundant.

Studies conducted in SMA patient cells in vitro showed that the activity of transcriptional oligonucleotides and the splice correcting oligonucleotide when mixed together is greater than either oligonucleotide alone. Human SMA fibroblasts were transfected with various concentrations of oligonucleotides using lipofectamine transfection. Full-length SMN2 mRNA was measured by quantitative RT-PCR assay while SMN protein levels were determined by ELISA. SMN RNA and protein levels in oligonucleotide treated cells were compared with untreated (lipofectamine only) controls. Multiple different transcriptional oligonucleotides directed against SMN-AS were tested and were effective to varying degrees. However, the effects of one oligo, namely Oligo 92, were superior to the other active oligos. Oligo 92 has a sequence set forth as CATAGTG(G)AAC(A)G(A)T (SEQ ID NO: 6). Nucleotides in parenthesis are 2′O methyl (TOME) and all other nucleotides are LNAs.

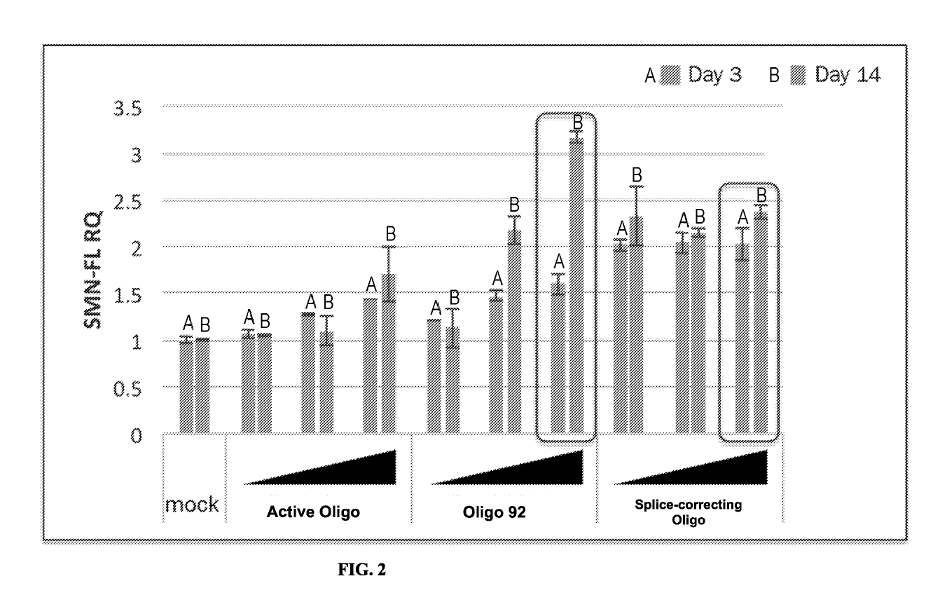

As shown in FIG. 2, both the transcriptional oligonucleotides and the splice correcting oligonucleotide showed upregulation of SMN2 mRNA and SMN protein. The splice-correcting oligonucleotide has a sequence set forth as TCACTTTCATAATGCTGG (SEQ ID NO: 7) with each nucleotide being a 2′-O-methoxyethyl (2′MOE) nucleotide.

The transcriptional oligonucleotides and the splice correcting oligonucleotide were combined in two different ways. Either the concentration of the transcriptional oligonucleotides was fixed at one concentration and a splice-correcting oligonucleotide was tested at a range of different concentrations or the transcriptional oligonucleotide concentration was varied and the concentration of the splice-correcting oligonucleotideₓ was fixed. In the case of multiple oligonucleotides (the combination of the two oligonucleotides) was superior to either oligonucleotide when tested alone. In some embodiments, transcriptional oligonucleotides that display strong transcriptional activation but induce less exon 7 inclusion may show more beneficial activity when combined with splice correcting oligonucleotides and small molecules that affect splicing.

### Example 2. Disrupting Interaction Between Long Non-Coding RNA (lncRNA) and PRC2 with Transcriptional Activating Oligonucleotides Enhances the Activity of SMN2 Splice Correcting Mechanism in Neurons

Primary cortical neurons were isolated, for example, from forebrains of E14 embryos obtained from pregnant mice 5025 wildtype (WT). The cortical neurons were then resuspended in culture media and seeded in 24 well plates. Cultures were treated with splice correctors (e.g., splice correcting oligonucleotides (SCO)) alone or in combination with transcriptional activators (e.g., transcriptional activating oligonucleotides) for about 14 days without any change in culture media. RNA or protein samples were then collected from individual wells and analyzed for SMN2 expression. Transcriptional activators are agents targeting PRC2-associated regions to inhibit the interaction of PRC2 with long RNA transcripts such that gene expression is upregulated or increased. Examples of such transcriptional activators that can be used to treat the neurons include but are not limited to transcriptional activating oligonucleotides as described herein. Splice correctors are agents that modulate SMN2 splicing to promote inclusion of exon 7 of the SMN2 pre-messenger RNA. Examples of such splice correctors that can be used to treat the neurons include but are not limited to splice correcting oligonucleotides as described herein.

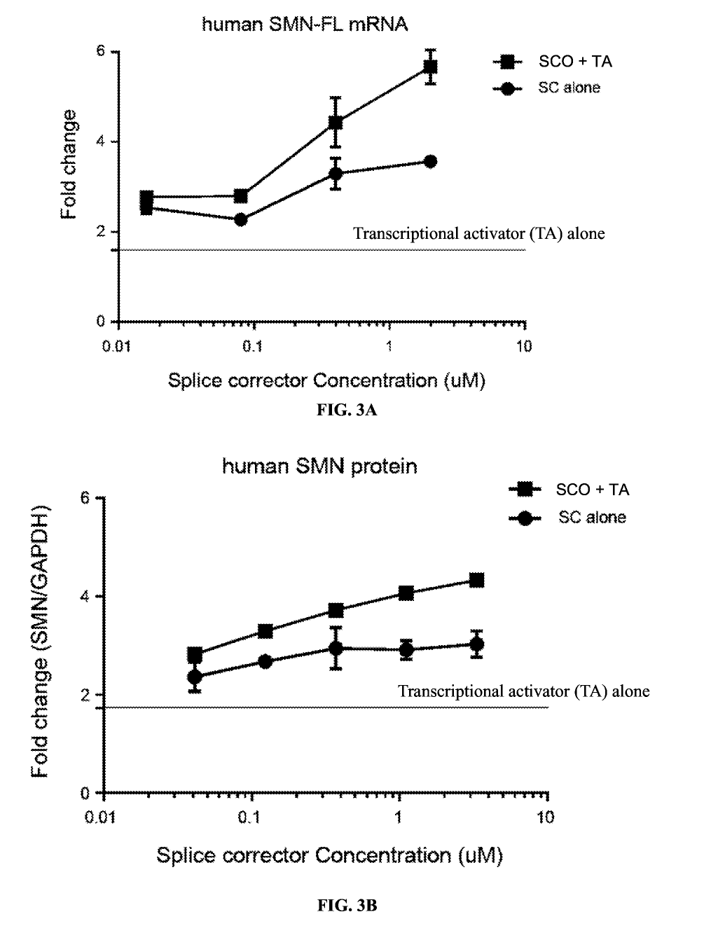

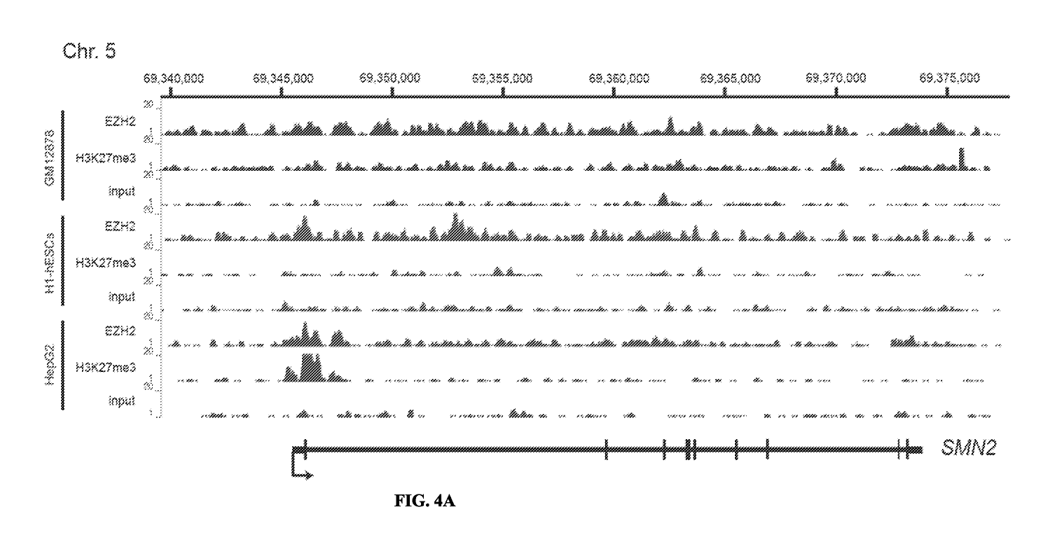

As shown in FIGS. 3A and 3B, the combination of the transcriptional activator (e.g., transcriptional activating oligonucleotides) and splice corrector (e.g., splice correcting oligonucleotides) showed enhanced upregulation of exon7-containing SMN2 mRNA and protein, as compared to treatment with splice correctors alone. Even at maximal upregulation of SMN2 expression observed at higher concentrations of the splice correctors, lncRNA:PRC2 disruption with transcriptional activators further increases SMN2 expression in neurons.

### Example 3. Gene Activation of SMN by Selective Disruption of lncRNA Recruitment of PRC2 for the Treatment of Spinal Muscular Atrophy

### Summary

Spinal muscular atrophy (SMA) is a neurodegenerative disease characterized by progressive motor neuron loss and caused by mutations in SMN1 (Survival of Motor Neuron 1). Currently, there is no disease-modifying therapy and the disease severity inversely correlates with the copy number of SMN2, a duplicated gene that is nearly identical to SMN1. The present disclosure delineates a novel mechanism of transcriptional regulation in the SMN2 locus. A previously uncharacterized long noncoding RNA, SMN-AS1, represses SMN2 expression by recruiting Polycomb Repressive Complex 2 (PRC2) to its locus. Using sterically blocking oligonucleotides to disrupt the interaction between SMN-AS1 and PRC2, the recruitment of PRC2 is inhibited while SMN2 expression is increased in primary neuronal cultures. Proof-of-concept evidence that SMA may be treatable by applying a novel gene-upregulation technology is demonstrated herein. Additionally, these data suggest that such approach can selectively upregulate genes that are epigenetically repressed by lncRNA and PRC2.

### Introduction

Polycomb Repressive Complex 2 (PRC2) is a histone methyltransferase complex that plays essential roles in development and disease (Di Croce and Helin, 2013; Simon and Kingston, 2013; Kadoch et al., 2016). Mammalian PRC2 is composed of four obligatory subunits, EED, SUZ12, RbAp48, and EZH1 or EZH2. EZH1 and EZH2 are the histone methyltransferases that confer the trimethylation of lysine 27 of histone H3 (H3K27me3) and PRC2-mediated H3K27me3 is associated with the maintenance of gene repression (Simon and Kingston, 2013). The formation of an EZH1- or EZH2-containing PRC2 complex depends on chromosomal location and cell type (Margueron et al., 2008). On its own, the core PRC2 complex does not contain any sequence-specific DNA binding activity. However, it interacts with other DNA-binding subunits in a substoichiometric manner and is recruited to specific Polycomb Response Elements (PREs) (Vizan et al., 2015). Despite only a few mammalian PREs identified to date (Sing et al., 2009; Woo et al., 2010, 2013; Basu et al., 2014), emerging data suggests that PRC2 interacts with a large number of RNA transcripts (Zhao et al., 2010; Davidovich et al., 2015) and a subset of which may aid PRC2 recruitment to specific genomic locations (reviewed by Davidovich and Cech, 2015) to repress the expression of neighboring genes. Combining a functional genomic approach and in-depth mining of epigenetic databases led to the identification of PRC2-regulated genes including SMN2, a disease-modifying gene for SMA.

Spinal Muscular Atrophy (SMA) is the leading genetic cause of infant mortality, caused by deletions or mutation of SMN1 which gene product is critical for mRNA processing (Rossoll et al., 2002). SMN1 is uniquely duplicated in the human genome and yields SMN2, which is nearly identical in sequence. However, a C-to-T point mutation in exon 7 of SMN2 results in preferential skipping of exon 7 during pre-mRNA splicing and production of a truncated and unstable protein. A small fraction (10-20%) of pre-mRNA transcribed from SMN2 is spliced correctly to include exon 7 and produces a full-length SMN (SMN-FL, inclusive of exon 7) that is identical to the SMN1 gene product (Monani, 2005; Vitte et al., 2007).

Spinal motor neurons are highly sensitive to SMN1 deficiency and their premature death causes motor function deficit in SMA patients (Monani, 2005; Burghes and Beattie, 2009). The SMN2-derived SMN-FL mRNA can extend spinal motor neuron survival yet insufficient level of SMN-FL mRNA eventually leads to cell death. Clinically, SMA patients who have increased SMN2 genomic copy number have a less severe disease phenotype (Lefebvre et al., 1997; Feldkotter et al., 2002). Therefore, it was thought that increasing SMN2 transcription could phenocopy the beneficiary effect of SMN2 gene amplification and compensate for SMN1 deficiency. In addition, SMN1 heterozygotes are asymptomatic while affected homozygotes have 10-20% of normal SMN levels, so it was predicted that a modest SMN2 upregulation would provide significant therapeutic benefit. Here, it is established that PRC2 interacts with a newly identified long noncoding RNA (lncRNA) transcribed within the SMN2 locus and regulates SMN2 expression through PRC2-associated epigenetic modulation. Furthermore, the selective upregulation of SMN2 expression by sterically blocking the lncRNA-mediated recruitment of PRC2 to the SMN2 locus is demonstrated.

### Results

### PRC2 Modulates SMN2 Expression

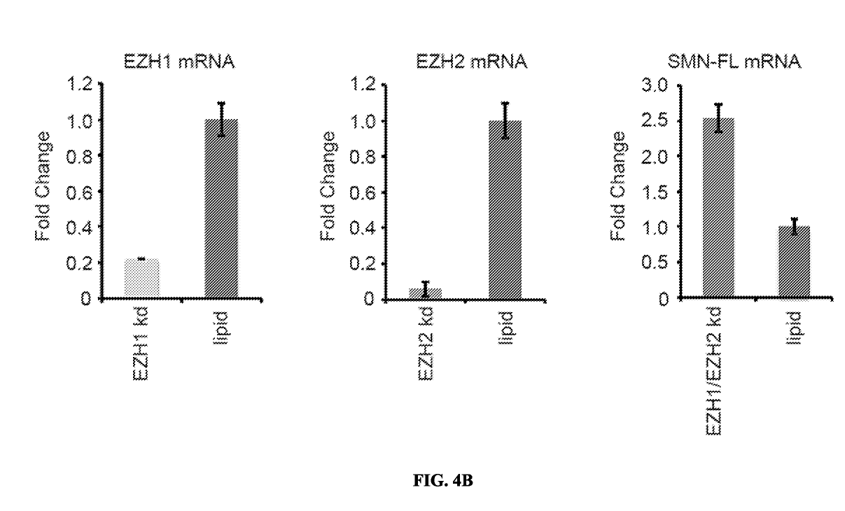

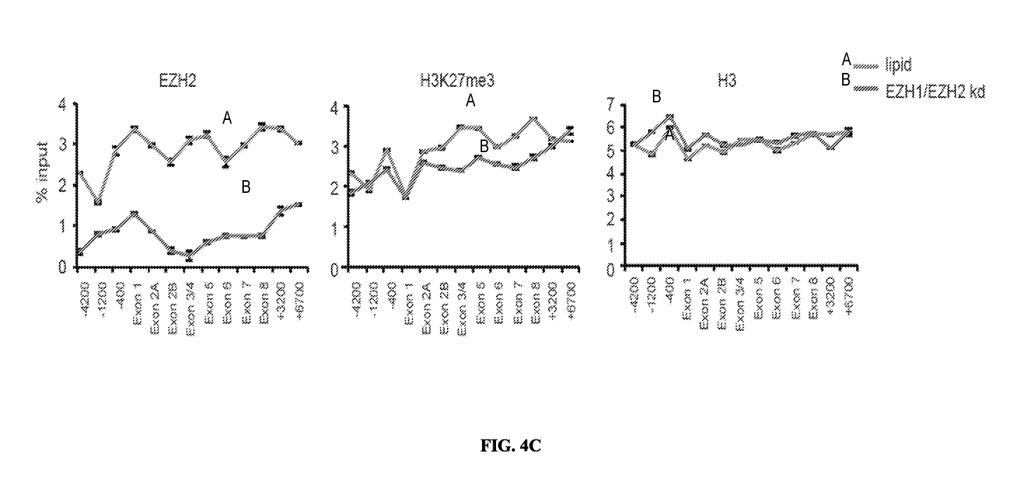

In depth and focused analysis of publically available chromatin immunoprecipitation (ChIP) sequencing data suggests that PRC2 is associated with SMN2 in multiple cell types (FIG. 4A). Enriched EZH2 interaction and its associated repressive chromatin marks,

H3K27me3, suggest that PRC2 activity is targeted to the gene. To determine whether disruption of PRC2 activity could lead to increases in SMN2 expression, EZH1 and EZH2 mRNAs were knocked down in SMA fibroblasts using antisense oligonucleotide (ASO) designed for RNaseH-mediated degradation. Two days post-transfection, there were significant decreases in EZH1 and EZH2 mRNA levels. Knockdown of both EZH1 and EZH2 in the SMA fibroblasts was associated with an increase in SMN-FL mRNA (FIG. 4B). The SMN1 and SMN2 loci (from here on collectively termed “SMN locus”) were further analyzed for chromatin changes upon EZH1/EZH2 knockdown through ChIP. Because SMN1 and SMN2 have >99% sequence identity (27,890 of 27,924 basepair match), it is not possible to distinguish between the two genes using this technique. The decreased association of EZH2 as well as decreased H3K27me3 levels at the locus were observed, without any changes in total H3 (FIG. 4C). This suggests that PRC2 directly regulates the expression of SMN.

### Identification of SMN-AS1 at the SMN Locus

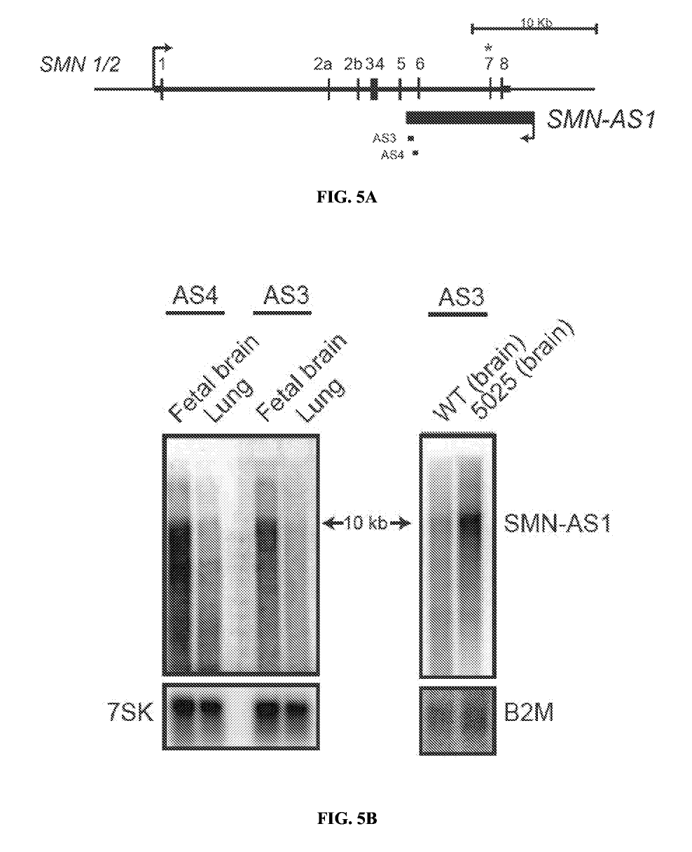

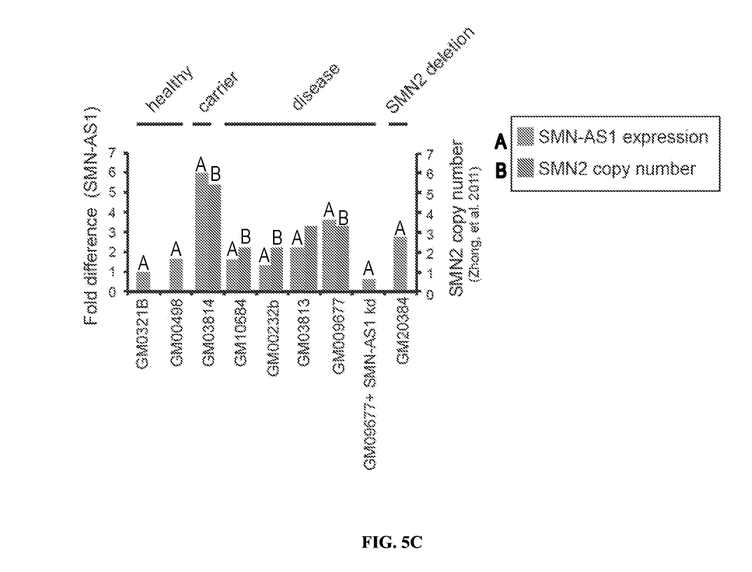

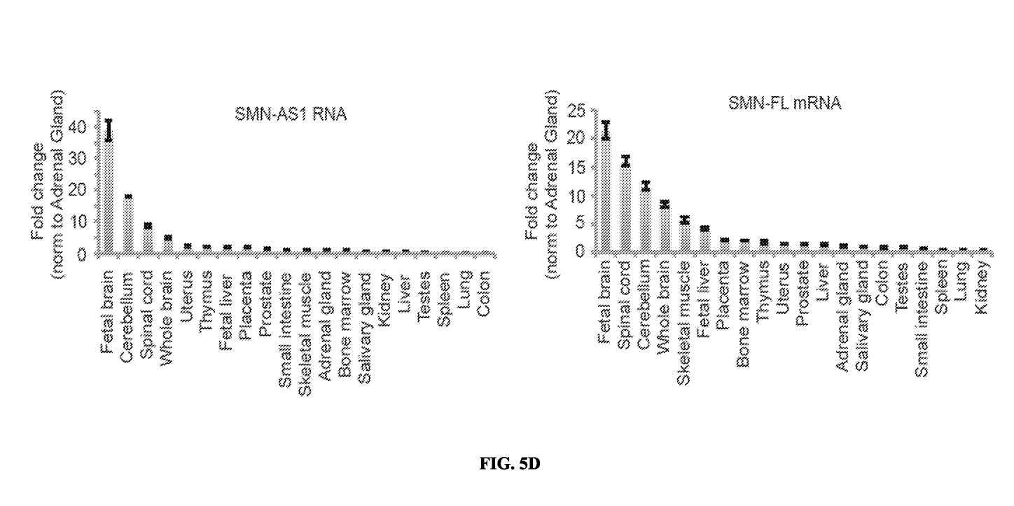

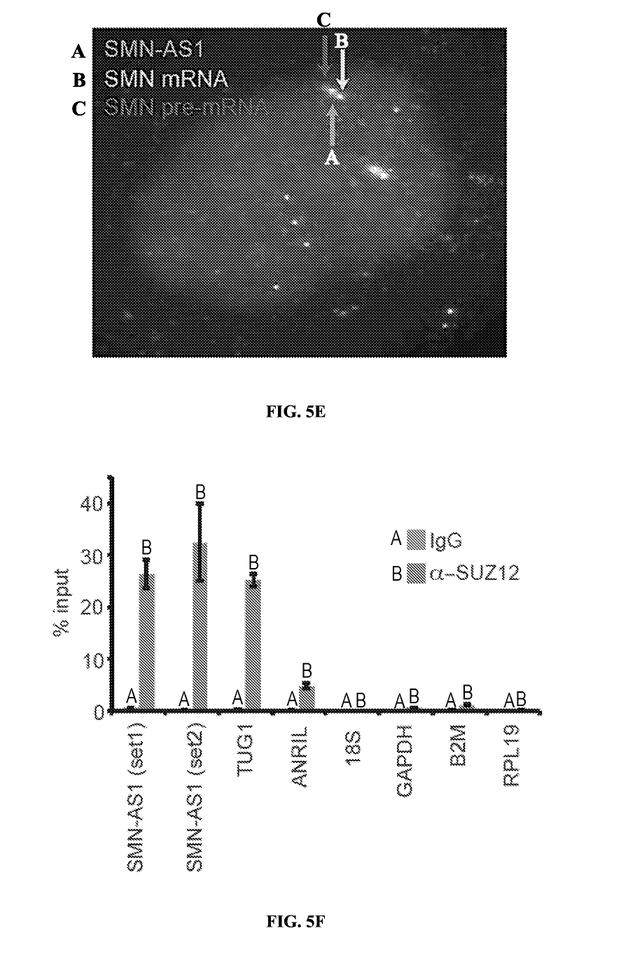

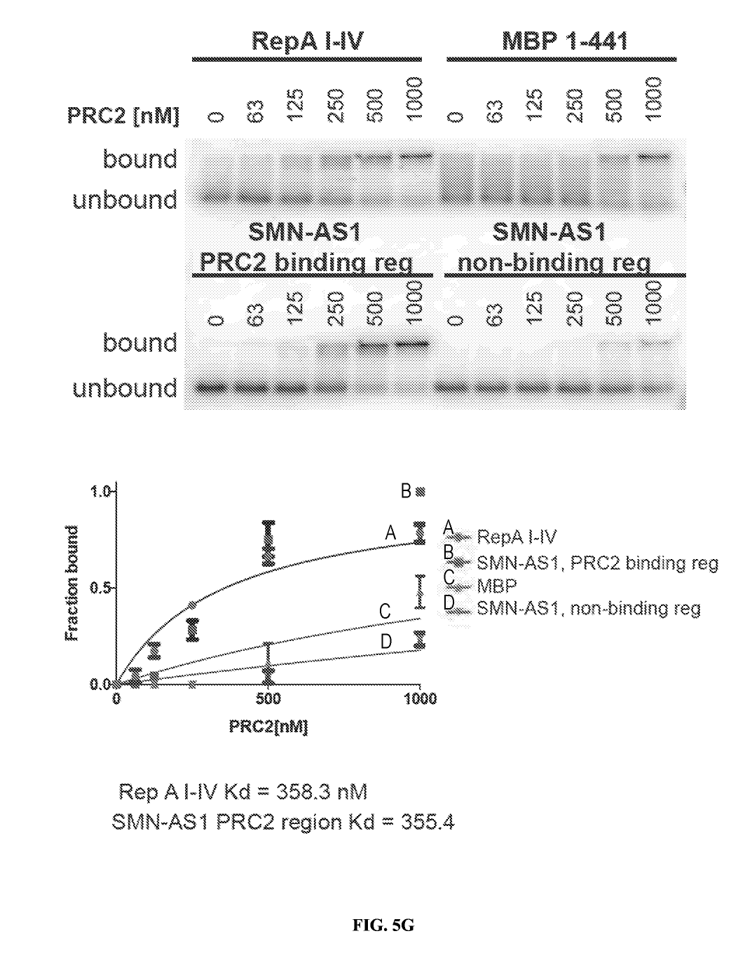

Detailed analysis of RNA immunoprecipitation (RIP)-seq datasets revealed a previously undescribed PRC2 interacting antisense RNA within the mouse Smn locus (Zhao et al., 2010). Whether the antisense transcript exists in human and may have a role in PRC2-mediated SMN repression was investigated. Next generation RNA-sequencing revealed that a lncRNA, SMN-AS1, is transcribed from the SMN loci (FIGS. 5A, 5C). Due to the high sequence identity between the SMN1 and SMN2 loci, the lncRNA, SMN-AS1 was expected to be transcribed from both loci. Furthermore, its expression in both SMN1- or SMN2-mutated cell lines was observed (FIG. 5C). Northern blot analysis of human fetal brain and adult lung tissues revealed that SMN-AS1 is up to 10 kb long, is heterogeneous in size, and has differential expression between the two tissue types (FIG. 5B). To confirm the specificity of the SMN-AS probe, a humanized SMA mouse model carrying two copies of the human SMN2 genomic locus (5025 strain) was used (Le et al., 2005). Comparing the brain tissues from wild type and 5025 mice, a similar set of transcripts in the SMN2-harboring transgenic mice and in the human fetal brain were observed (FIG. 5B). By reverse transcription quantitative PCR (RT-qPCR), SMN-AS1 was detected in patient cell lines and the level of expression correlated with SMN2 copy number (FIG. 5C). In addition, it was found that SMN2 mRNA and SMN-AS1 expression is highly correlated with CNS tissues (FIG. 5D). Finally, strand-specific single-molecule RNA-fluorescent in situ hybridization (RNA-FISH) detected the SMN-AS1 at the SMN locus (FIG. 5E). Together, these data demonstrate the presence of an antisense transcript within the SMN locus.

SMN-AS1 binds PRC2

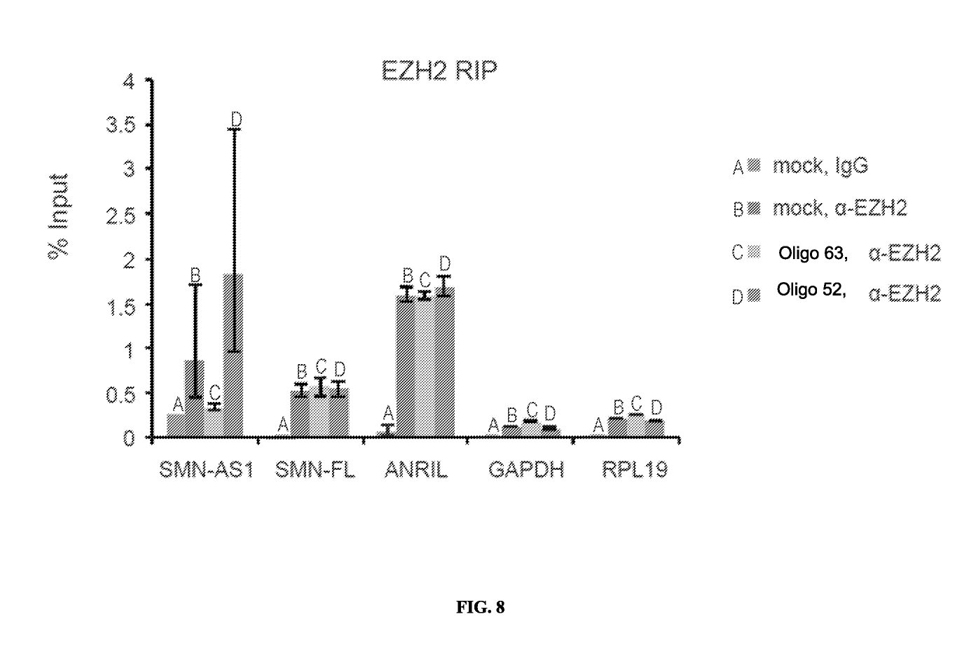

To investigate the role of SMN-AS1 in the PRC2-mediated epigenetic regulation of the SMN2 gene, native RIP (nRIP) was performed using an antibody against the PRC2 subunit, SUZ12, followed by RT-qPCR with 2 distinct probe sets directed to different regions of SMN-AS1. RIP-qPCR showed that SMN-AS1 is strongly associated with PRC2 in SMA fibroblasts (FIG. 5F). The association was stronger than, or comparable to, that of well-established PRC2 interacting lncRNAs including TUG1 (Zhang et al., 2014) and ANRIL (Kotake et al., 2011). Additionally, PRC2 did not associate with the abundantly expressed negative controls such as GAPDH and RPL19. Similar results were observed with the nRIP for EZH2 (FIG. 8) further supporting the association of SMN-AS1 with PRC2. Because nRIP identifies both direct and indirect interactions, RNA electrophoretic mobility shift assays (RNA EMSA) were performed to specifically detect direct interactions. Using a 441-nucleotide (nt) RNA containing the PRC2 interacting region of SMN-AS1 (SMN-AS1, PRC2 binding region) as identified by RIP-seq (Zhao et al., 2010), it was observed that purified recombinant human PRC2 (EED/SUZ12/EZH2) specifically changed the migration of this region of SMN-AS1 (FIG. 5G). Binding was concentration-dependent and was as robust as that of the 434-nt RepA RNA, a conserved domain of Xist RNA that is a well-documented PRC2-interacting lncRNA (Zhao et al., 2010; Cifuentes-Rojas et al., 2014). Dissociation constants (Kd) of both transcripts were estimated to be 350-360 nM. As specificity controls, a low level of background binding to a non-PRC2 interacting 441-nt region of the SMN-AS1 transcript (SMN-AS1, non-binding region) and to another non-specific mRNA of similar length, maltose-binding protein (MBP) from E. coli (Cifuentes-Rojas et al., 2014) was observed. These data demonstrate that SMN-AS1 lncRNA interacts directly and specifically with PRC2.

### Blocking PRC2:SMN-AS1 Interaction Upregulates SMN2 and Produces Epigenetic Changes

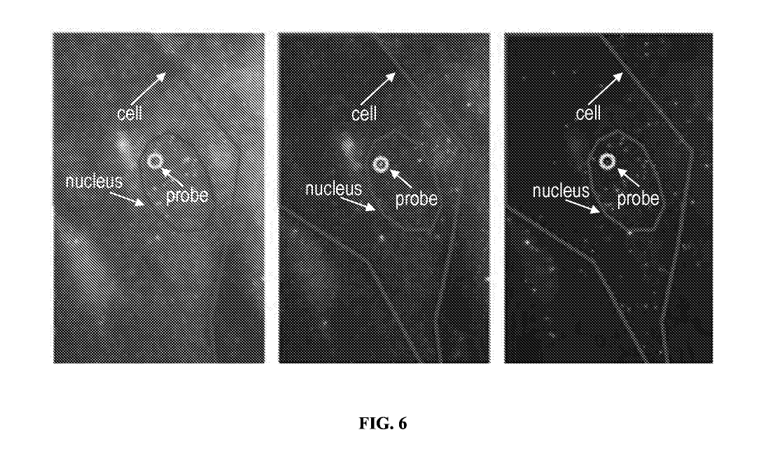

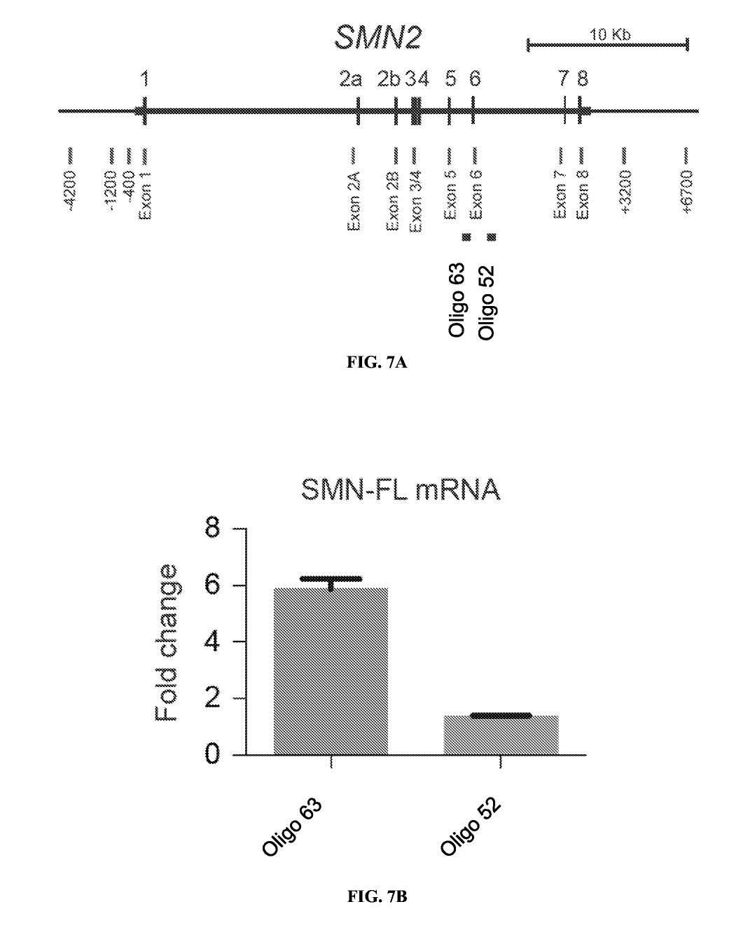

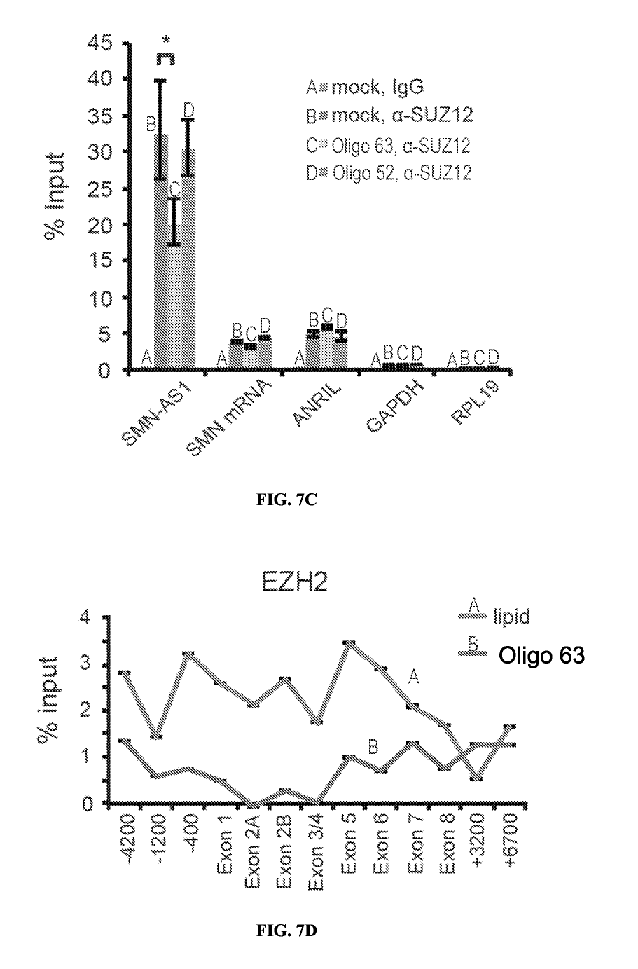

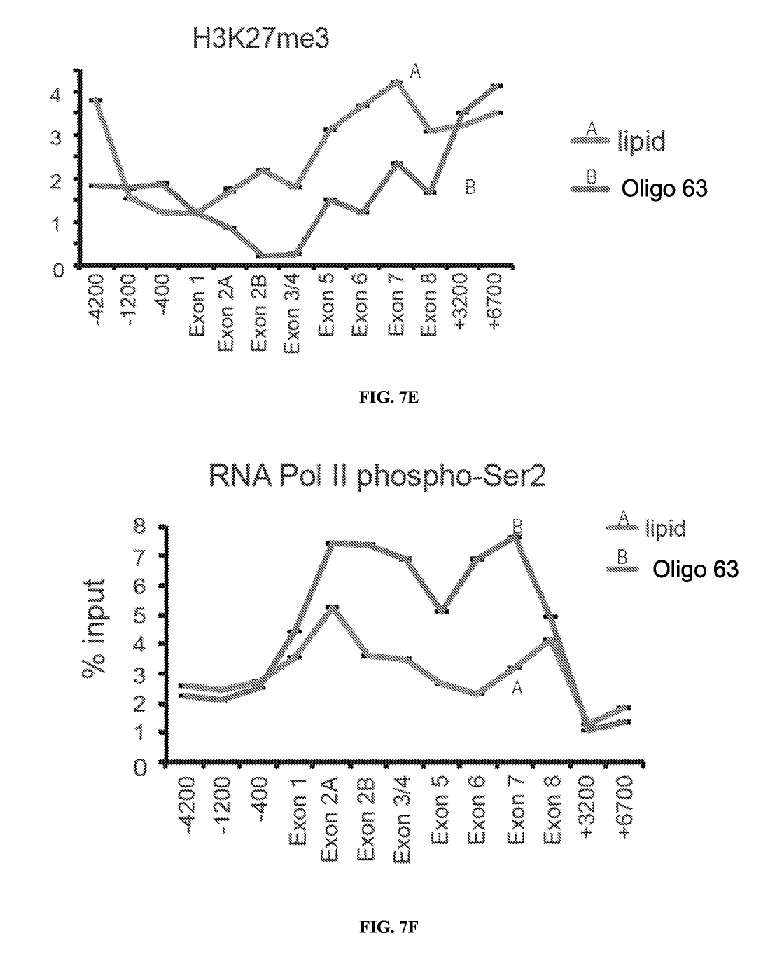

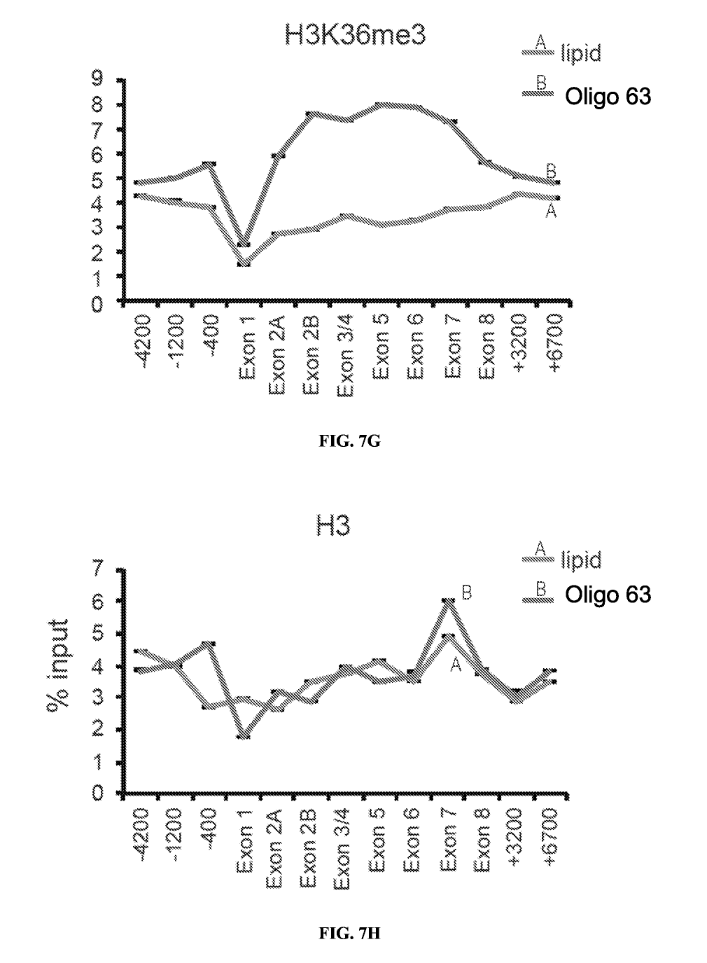

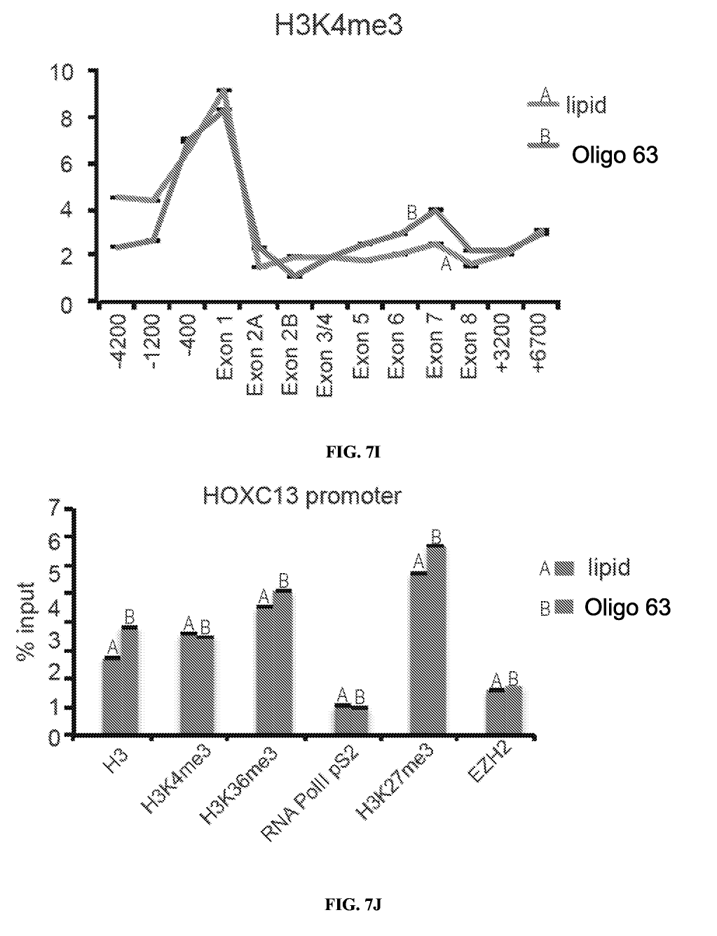

To investigate the effect of disrupting PRC2:SMN-AS1 interaction, ASOs targeting the PRC2-binding site of the lncRNA were designed. ASOs hybridize to target RNA sequences via Watson-Crick complementarity pairing. Depending on the arrangement of DNA- and LNA-modified nucleotides, such interaction can lead to either RNaseH-mediated degradation of target RNAs or hindrance of the interaction between target RNAs and their binding partners. For RNaseH-mediated degradation, a “gapmer” formatted ASO composed of a central DNA segment greater than 6 nucleotides (i.e. gap) flanked by 2 to 4 locked nucleic acid (LNA)-modified nucleotides is required. These gapmer ASOs were used to knockdown EZH1 and EZH2 in earlier experiments (FIG. 4C). In contrast to the gapmer arrangement, a “mixmer”-formatted ASO lacks the central DNA segment and does not support the RNaseH-mediated degradation mechanism. Instead, the binding of a mixmer ASO prevents the interaction between target RNA and its RNA or protein binding partners (Kauppinen et al., 2005). Mixmer ASOs consisting of LNA interspersed with 2′-O-methyl nucleotides (2′-OMe) for high-affinity binding to SMN-AS1 were generated. Screening multiple mixmer ASOs led to a focus on one efficacious mixmer ASO, Oligo 63 (FIG. 7A). Transfecting Oligo 63 into SMA fibroblasts significantly increased SMN-FL expression where as transfecting with another mixmer ASO, Oligo 52, which was not predicted to sterically block PRC2 recruitment, did not change SMN-FL expression (FIG. 7B). Consistently, nRIP showed that Oligo 63, but not Oligo 52, disrupted the binding of PRC2 to SMN-AS1, as shown by RIP-qPCR (FIG. 7C). Furthermore, no effect of Oligo 63 or Oligo 52 was observed on ANRIL, GAPDH, or RPL19 control RNAs. These results were also observed when the nRIP was performed using an antibody against EZH2 (FIG. 8). As expected, single molecule RNA-FISH for the localization of SMN-AS1 after transfection with Oligo 63 showed no change in both the abundance and the localization of SMN-AS1 in 93% of cells examined (39 of 42 nuclei) (FIG. 6). Together, these results demonstrate that selective inhibition of PRC2:SMN-AS1 interaction by a mixmer ASO leads to increase SMN2 expression.

To provide molecular insight on how the active mixmer induced SMN expression, the chromatin changes at the SMN locus in response to the disruption of PRC2:SMN-AS1 interaction was characterized using CUP analysis. When treated with Oligo 63, a loss of EZH2 association as well as decreased H3K27me3 levels, the histone mark associated with PRC2 activity with the SMN gene body, were observed (FIGS. 7D-7E). Thus, the mixmer ASO could indeed block the recruitment and activity of PRC2 at the SMN2 locus. Concomitantly, there was an increase in binding of RNA Pol II-phosphoSer2 (RNA Polymerase II, phosphorylated at serine 2) and elevated levels of H3K36me3, both of which indicate transcriptional elongation (FIGS. 7F-7G). By contrast, pan-H3 levels were similar amongst all samples (FIG. 7H). Moreover, H3K4me3, a mark of transcription initiation, did not change at the promoter (FIG. 7I), suggesting that the regulation is occurring at the transcriptional elongation level. No changes in PRC2 association were observed at another well-established Polycomb target locus, HOXC13, upon treatment (FIG. 7J). It was concluded that PRC2 recruitment and activity at the SMN locus can be selectively inhibited by sterically blocking the specific PRC2:SMN-AS1 interaction.

### Blocking PRC2 Recruitment Results in SMN2 Upregulation in Fibroblasts

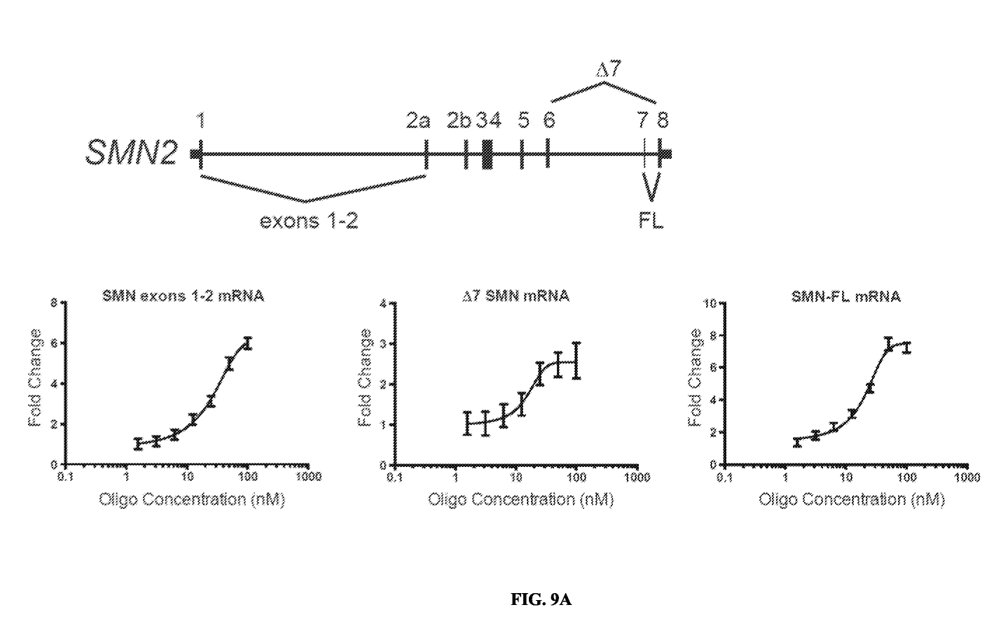

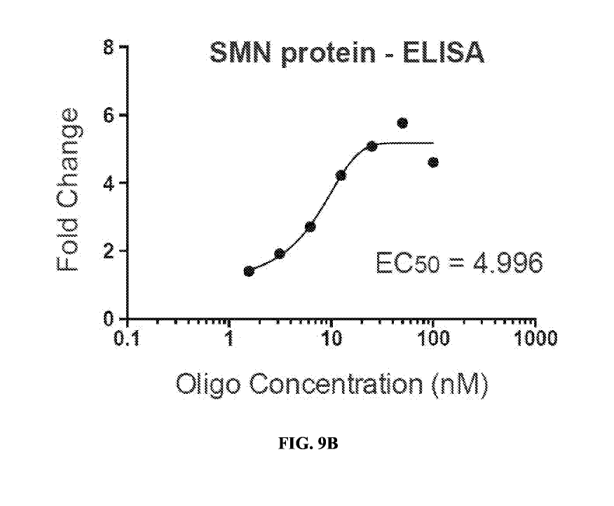

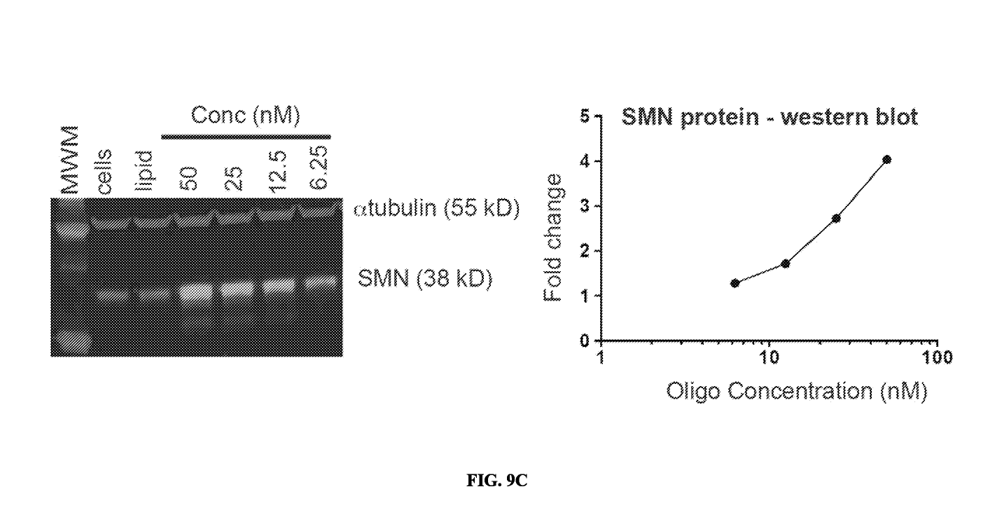

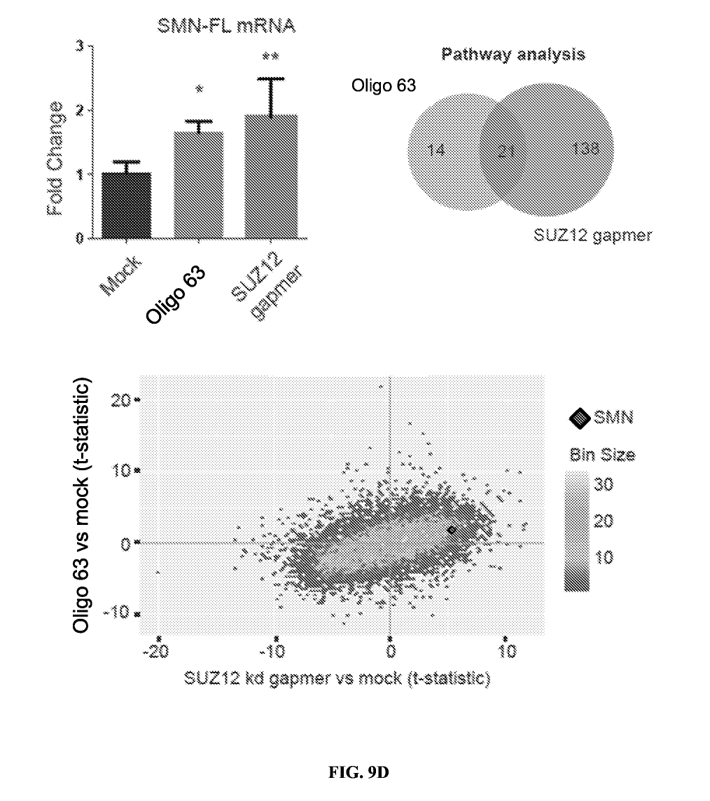

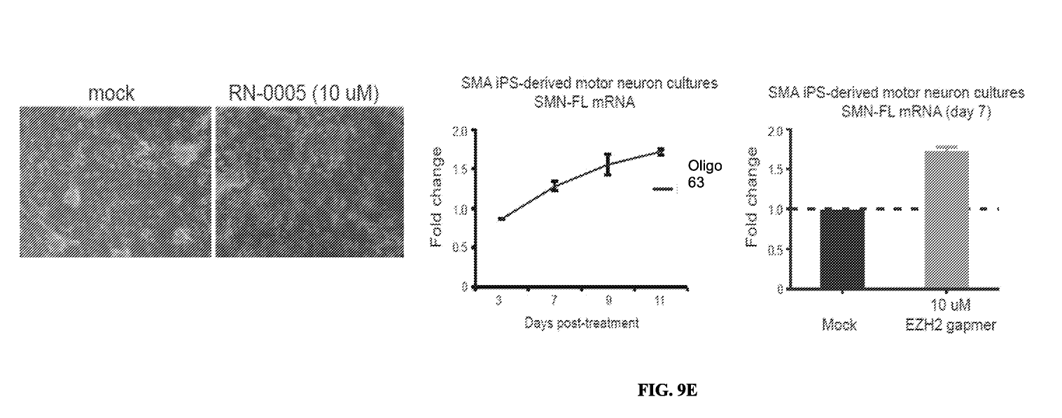

SMN2 mRNA upregulation resulting from the disruption of the PRC2:SMN-AS1 interaction and the subsequent epigenetic changes at the SMN locus were further characterized. The SMA fibroblast line GM09677, which carries two copies of the SMN2 gene and is homozygous for SMN1 exons 7 and 8 deletion, was used. Consistent with the transcriptional activation mechanism, RT-qPCR analyses with a few primer sets detect a concentration-dependent increase of various SMN mRNA transcripts, including all SMN isoforms (exon 1-2) as well as isoforms including or excluding exon 7, SMN-FL, and SMNΔ7, respectively (FIG. 9A). In agreement with this, overall SMN protein levels also increased, as shown by ELISA after 5 days of treatment (FIG. 9B). Western blotting revealed that this increase could be attributed to the 38-kDa SMN protein (FIG. 9C). ELISA and Western analyses both indicated up to 4-fold protein upregulation following treatment in SMA fibroblasts. Taken together, blocking the interaction of PRC2 with its recruiting lncRNA resulted in upregulation of both SMN mRNA and protein.

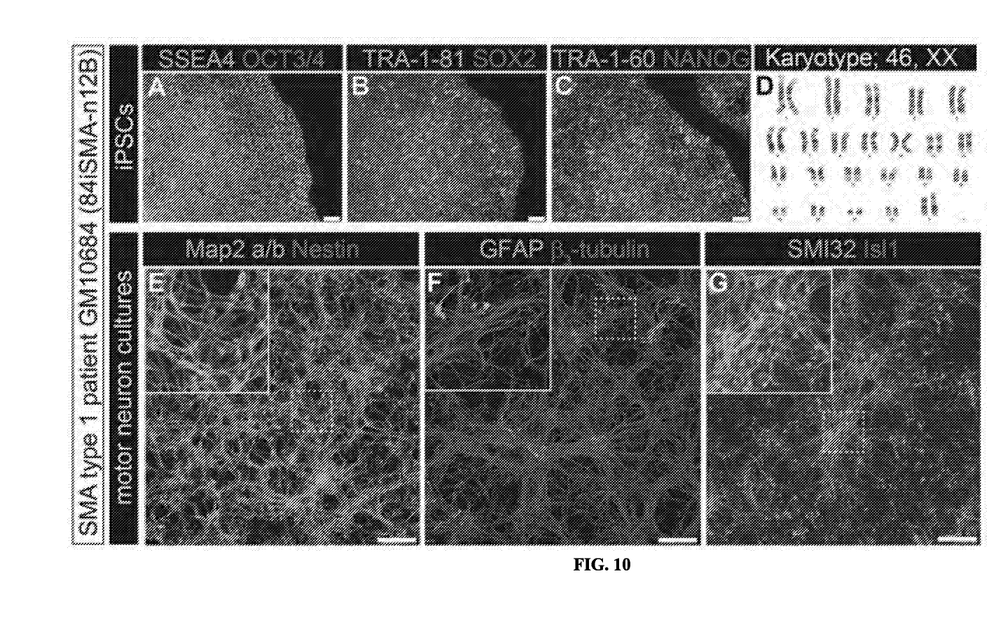

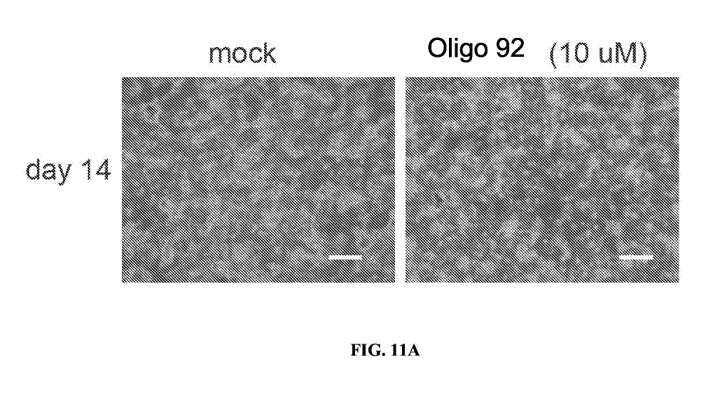

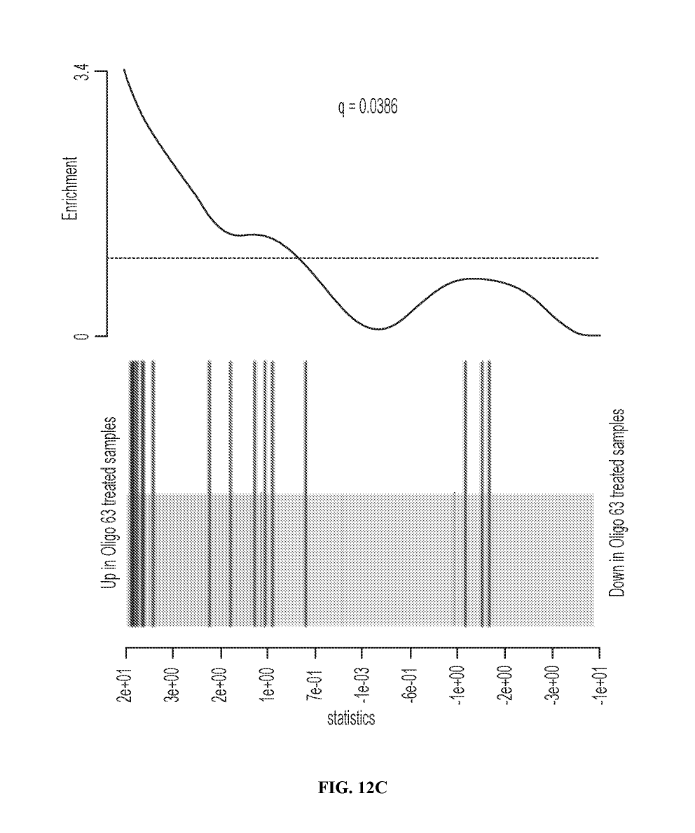

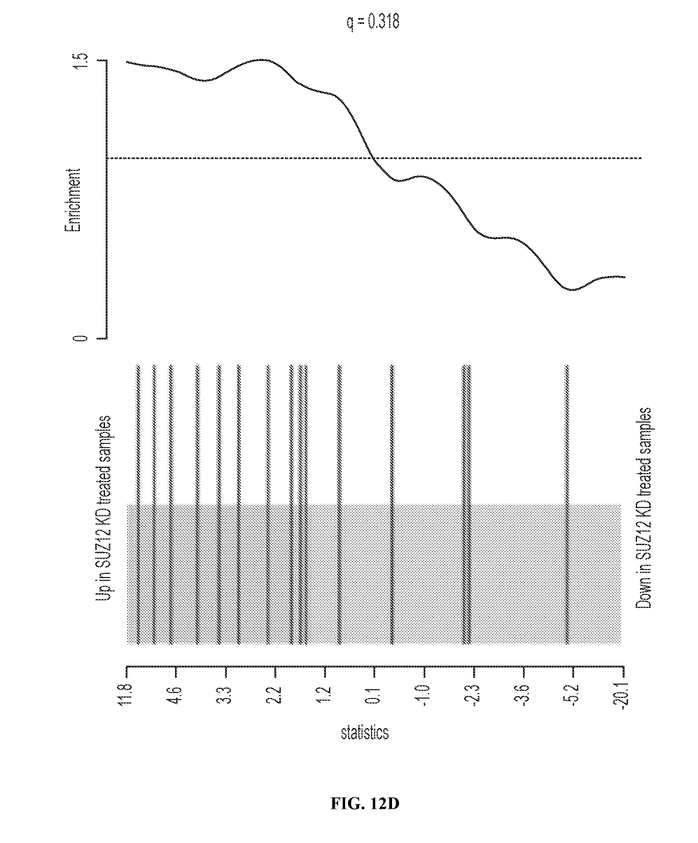

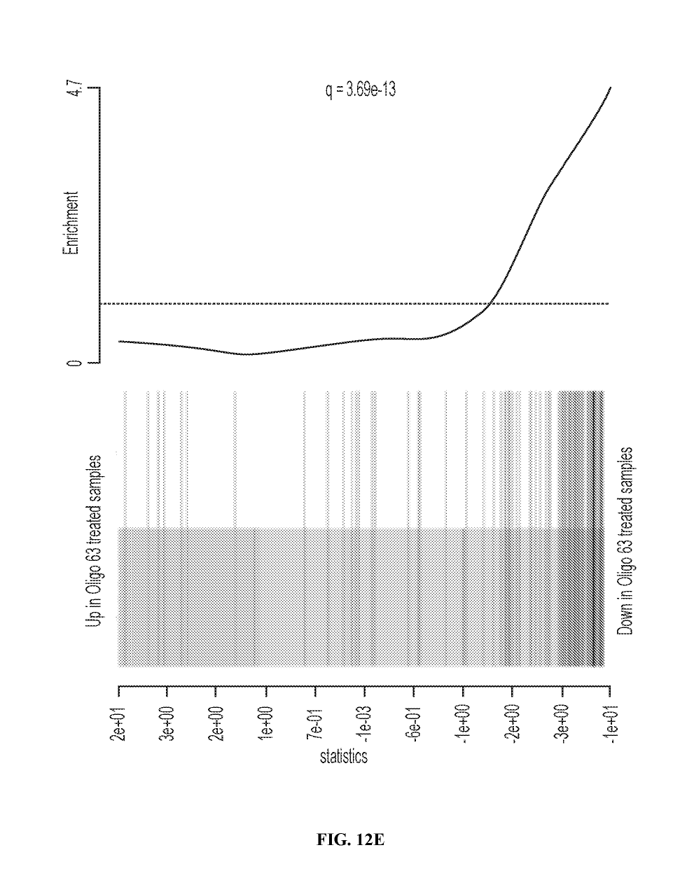

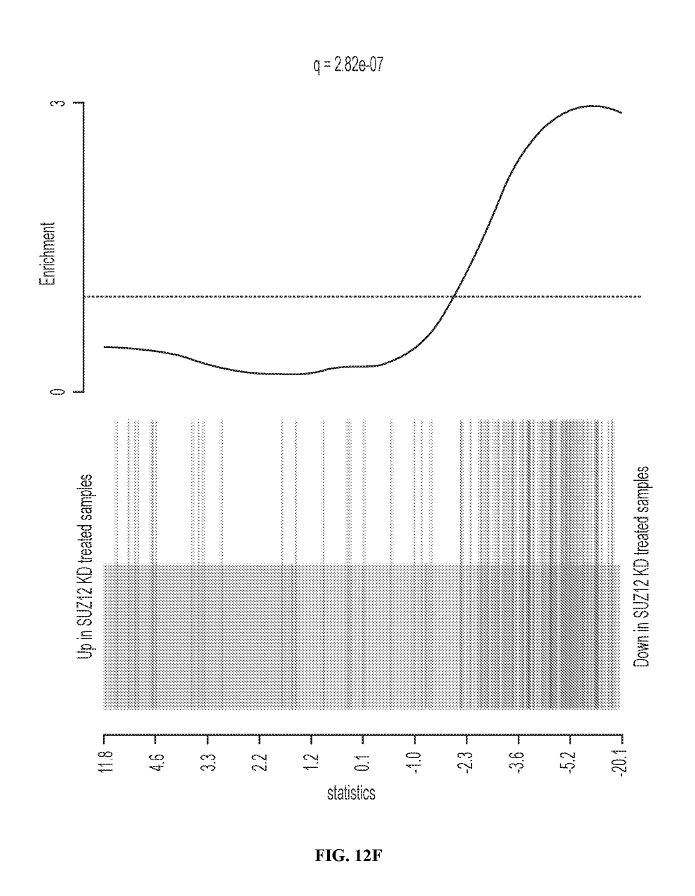

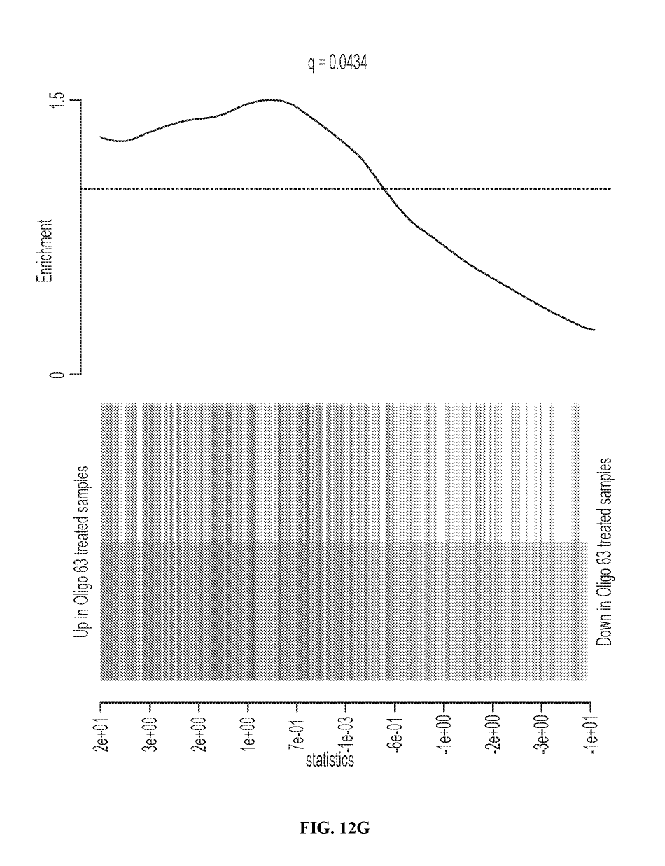

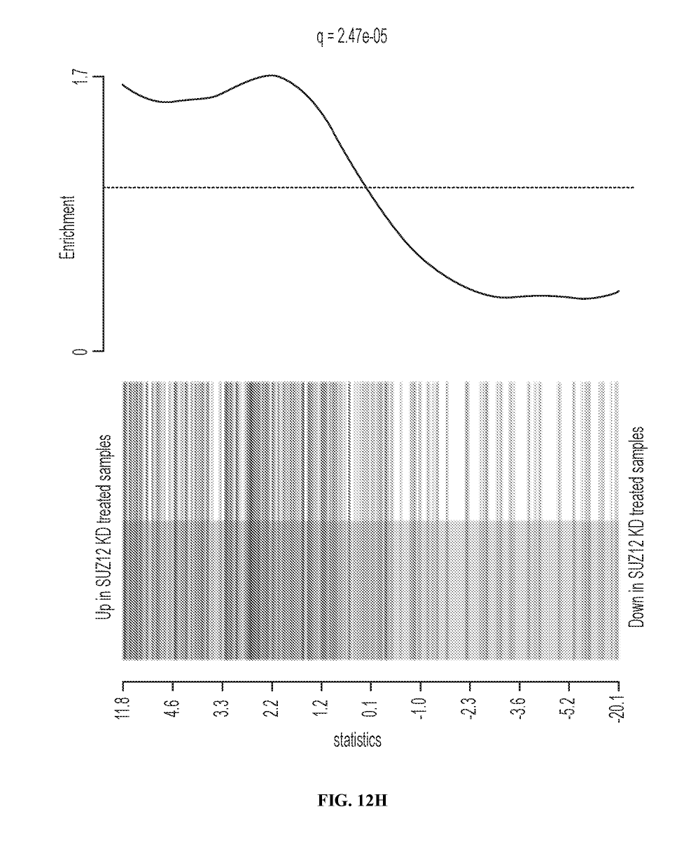

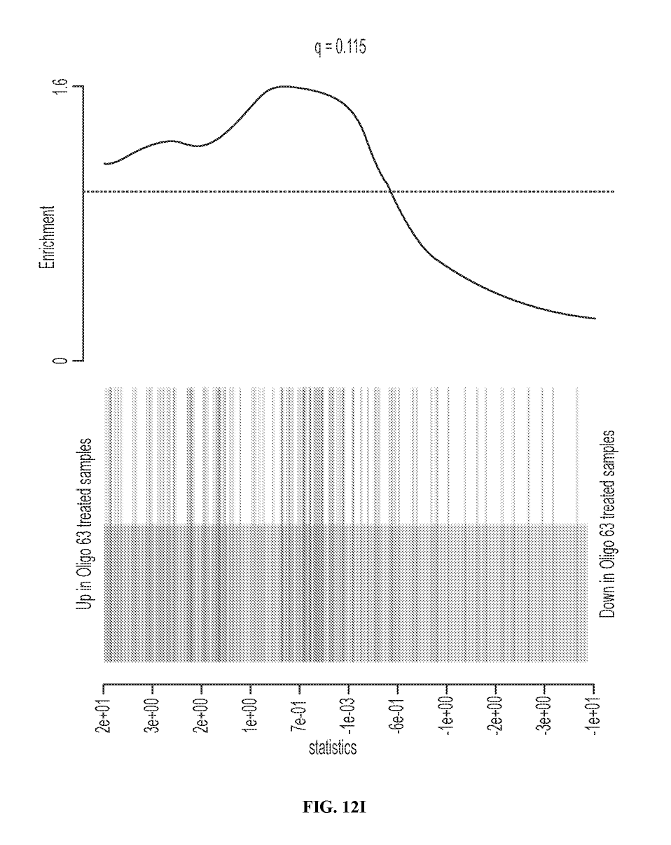

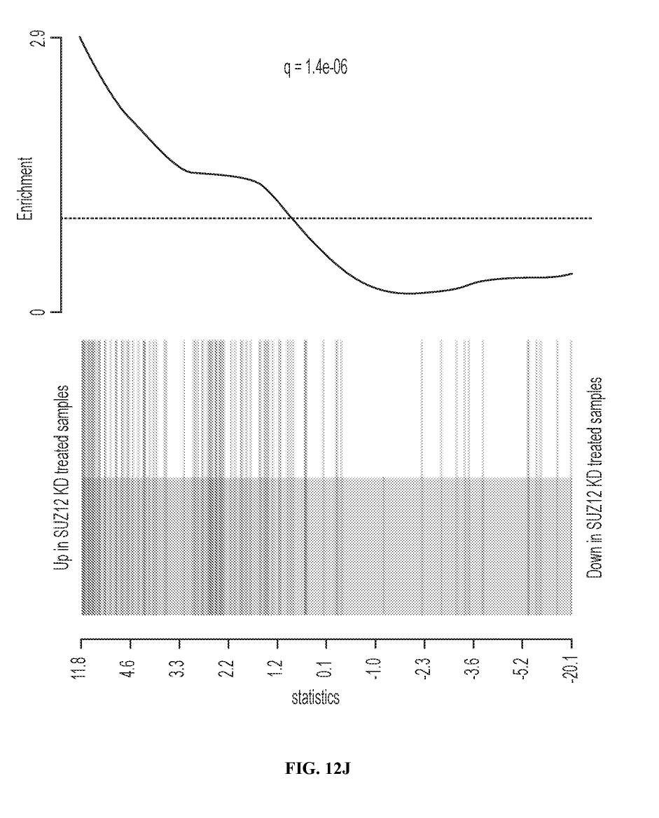

To determine how targeting the disruption of PRC2:SMN-AS1 interactions might affect PRC2 targets globally, RNA-sequencing was performed after transfection of the mixmer oligo, Oligo 63, or a gapmer ASO targeting SUZ12, a subunit of the PRC2 complex in SMA fibroblasts. Treatment with either the mixmer oligo or the SUZ12 gapmer ASO for 2 and 3 days resulted in significant increases in SMN mRNA levels compared to transfection control samples by RT-qPCR (FIG. 10B). Globally, there were approximately four-fold more gene expression changes with the SUZ12 gapmer ASO treatment than with Oligo 63 treatment that had at least a 1.5 fold change (q<0.05) as depicted by a scatterplot of the moderated t-statistics of the gene expression changes that occurred with oligo treatments. Focusing more locally by examining the nearest neighboring genes changing significantly in response to Oligo 63 treatment, the closest differentially expressed genes upstream (ADAMTS6) and downstream (BDP1) of SMN2 are 4.6 Mb and 1.4 Mb away, respectively. The nearest significant neighbor genes that changed after SUZ12 kd were TAF9, 0.8 Mb upstream, and BDP1, 1.4 Mb downstream, of SMN2. Pathway gene set analyses identified significant pathways (q<0.1) with each oligo treatment. While there was overlap between the oligo treatments, many more pathways changed separately with SUZ12 knockdown (FIG. 10B and FIG. 12A-J).

### Blocking PRC2 Recruitment Results in SMN2 Upregulation in Neuronal Cultures

While SMN expression is ubiquitous, its expression is highest in the central nervous system (FIG. 5D) (Boda et al., 2004), particularly in spinal motor neurons where the disease is manifested (Battaglia et al., 1997; Monani, 2005; Burghes and Beattie, 2009). To assess the activity of Oligo 63 in disease-relevant cell types, SMN expression in two neuronal cell types was examined. First, induced pluripotent stem cells (iPSC) derived from SMA patient fibroblasts were generated and differentiated into SMI32+ motor neurons (FIG. 10). After treating with an activating ASO, SMN-FL mRNA increased 1.8-fold relative to untreated motor neurons (FIG. 9E). As expected, EZH2 knockdown also led to similar increase in SMN-FL mRNA. The delayed increase in human SMN-FL mRNA levels in neurons relative to fibroblasts may be partially due to the mode of delivery (unassisted delivery versus transfection) and/or the non-proliferating state of the neuronal cells versus the highly proliferative fibroblasts. Consistent with the latter, the rate of H3K27me3 removal from the chromatin of non-dividing cells is slower than in proliferating cells (Agger et al., 2007). Taken together, these data show that disrupting the PRC2:SMN-AS1 interaction leads to SMN upregulation in disease-relevant and post-mitotic neuronal cells.

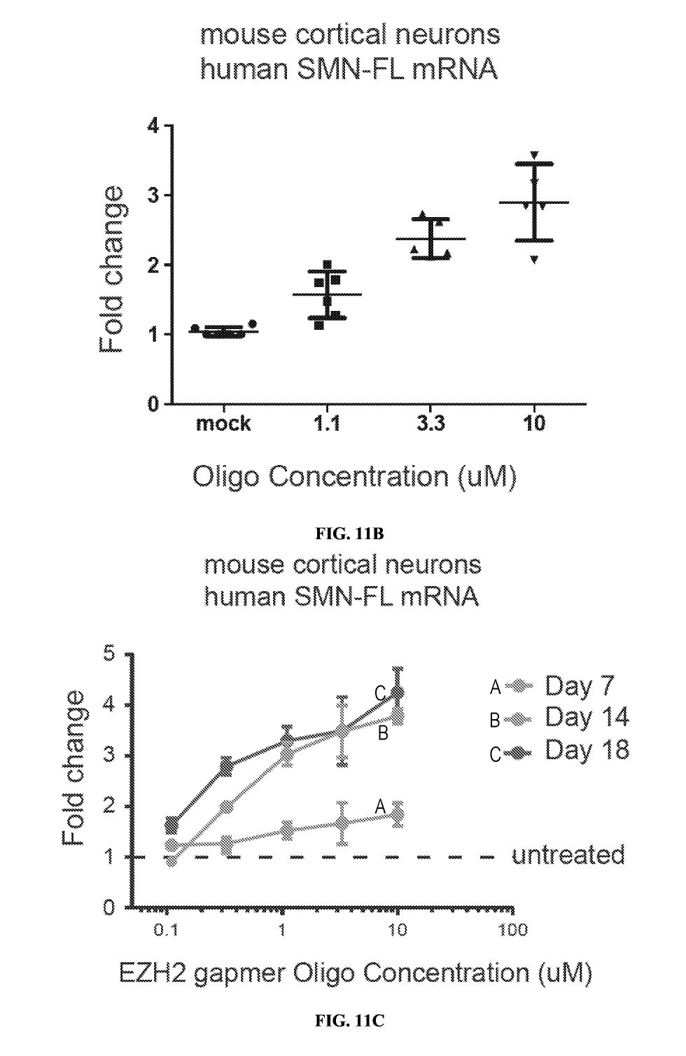

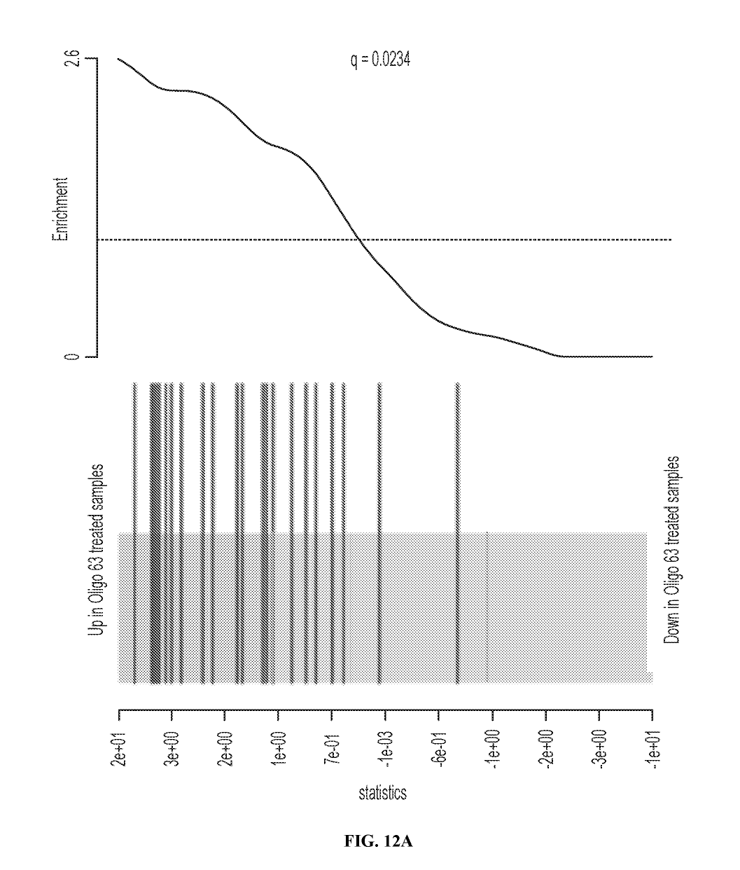

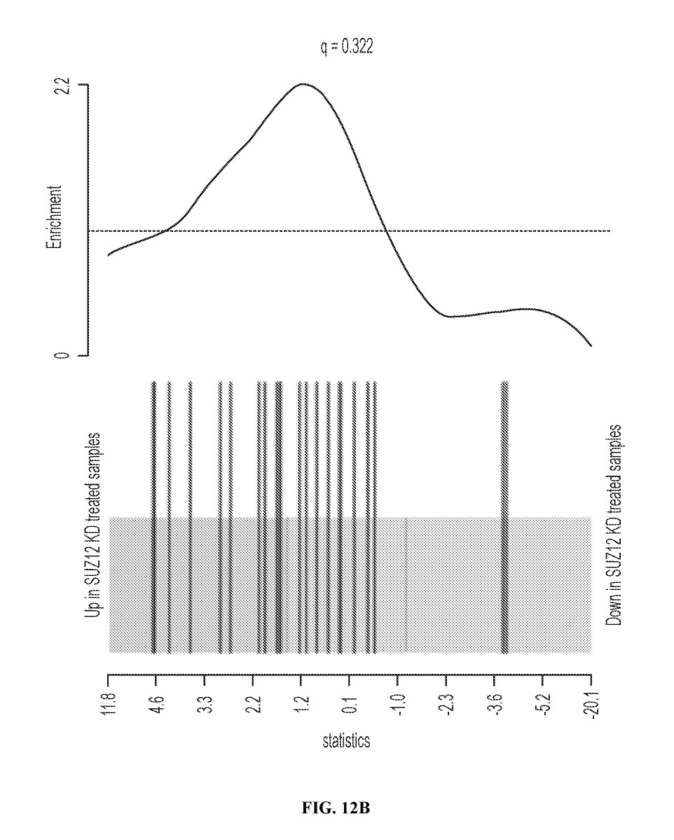

Primary cortical neuronal cells from E14 embryos of the 5025 SMA mice were also prepared and then treated with a chemical variant of Oligo 63 that targets the same SMN-AS1 sequence and has more favorable in vivo safety profile. Oligo 92 was added to culture medium at 1.1, 3.3, and 10 μM for 14 days without obvious toxicity or changes in cell morphology (FIG. 11A). A concentration-dependent increase in SMN-FL mRNA with a 3-fold increase at 10 μM following 14 days of treatment was observed (FIG. 11B). Consistent with the results obtained from patient fibroblasts (FIG. 4B), cortical neurons treated with an EZH2 gapmer ASO resulted in a concentration-dependent increase in SMN-FL mRNA levels (FIG. 11C). Several other unrelated ASOs were tested and changes in SMN-FL levels were not observed. The findings from ex vivo cortical neurons lend additional support to the transcriptional activation mechanism in terminally differentiated neuronal cells.

Combination of Transcriptional Upregulation and Splice Correcting Oligo Increases SMN-FL mRNA

Splice correcting modifiers are designed to facilitate the inclusion of exon 7 during splicing of SMN2 mRNA. Consequently, SMN-FL mRNA and functional SMN protein containing exon 7 would be produced. While steady-state total SMN mRNA levels would not increase with a splice correcting modifier, the shift to increase SMN-FL mRNA levels has been demonstrated to be beneficial to survival in mice (Hua et al., 2010; Palacino et al., 2015) and in humans (Chiriboga et al., 2016). Since the transcriptional activation approach upregulates SMN through a distinct mechanism from that of a splice corrector, it was thought that combining these two mechanisms would be more effective than either one of the two approaches alone. The 5025 cortical neurons were treated with either a splice correcting ASO (SCO), a transcriptional activating mixmer ASO (Oligo 92), or a combination of the two ASOs for 14 days to measure the levels of SMN-FL mRNA (FIG. 3A). While treatment with the SCO alone resulted in a 2-3-fold increase with the SCO, an additional 1.8-fold increase was observed in the presence of Oligo 92. This effect was also observed with increases in the human SMN protein levels by a human-specific ELISA (FIG. 3B). Whether the transcriptional activating mixmers affected mouse smn levels were previously tested and no changes were observed, as expected, because the transcriptional activating mixmer does not target any sequence within the mouse smn locus. While the SCO upregulated SMN-FL protein levels approximately 2.5-fold, the combination resulted in the increase of SMN levels to 4-fold. These data provide evidence that Oligo 92 increases SMN-FL mRNA and SMN protein levels by a mechanism that is independent and complementary to that of a SCO.

### Discussion

There is presently no approved disease-modifying therapeutic for SMA and treatments are focused on addressing symptoms ranging from respiratory complications to muscle atrophy. Currently, different approaches to treat SMA are being tested in clinical trials, most of which utilize splice correction mechanism to include exon 7 of SMN2 (reviewed by Cherry and Androphy, 2012). Distinct from the splice correction approach, a novel transcriptional upregulation method to selectively upregulate endogenous SMN mRNA and protein is reported. The overall changes in PRC2 and RNA Polymerase II occupancy, and histone modifications suggest that the increase in steady state levels of SMN2 arises at the transcriptional level. Indeed, when mouse primary cortical neurons were treated with the transcription activating ASO and a splice correcting oligo, an enhanced effect of SMN-FL mRNA and protein beyond that offered by a splice-correcting therapy alone was observed, which may potentially confer greater therapeutic benefit.

### Materials and Methods

Oligo Sequences.

The sequences of the oligos tested are shown in Table 1. All oligos in Table 1 are fully phosphorothioated with the exception of Oligo 69, which has the same base sequence as Oligo 92, but has a 50/50 mix of phosphorothioate and phosphodiester linkages.

TABLE 1
Oligo sequences

| Oligo | SEQ ID | Base | |
|---|---|---|---|
| Name | NO | Sequence | Full sequence with chemical modifications |
| Oligo 52 | 26 | AGAUGCAGT | lnaAs; omeGs; lnaAs; omeUs; lnaGs; omeCs; lnaAs; omeGs; |
| | | GCUCUT | lnaTs; omeGs; lnamCs; omeUs; lnamCs; omeUs; lnaT |
| Oligo 63 | 27 | CATAGUGGA | lnamCs; omeAs; lnaTs; omeAs; lnaGs; omeUs; lnaGs; omeGs; |
| | | ACAGAT | lnaAs; omeAs; lnamCs; omeAs; lnaGs; omeAs; lnaT |
| Splice | 28 | TCACTTTCA | moeTs; moemCs; moeAs; moemCs; moeTs; moeTs; moeTs; |
| corrector | | TAATGCTGG | moemCs; moeAs; moeTs; moeAs; moeAs; moeTs; moeGs; |
| | | | moemCs; moeTs; moeGs; moeG |
| Oligo 92 | 29 | CATAGTGGA | lnamCs; lnaAs; lnaTs; lnaAs; lnaGs; lnaTs; lnaGs; omeGs; |
| | | ACAGAT | lnaAs; lnaAs; lnamCs; omeAs; lnaGs; omeAs; lnaT |
| Oligo 69 | 29 | CATAGTGGA | lnamCs; lnaAs; lnaTs; lnaAs; lnaGo; lnaTo; lnaGo; omeGo; |
| | | ACAGAT | lnaAo; lnaAo; lnamCo; omeAs; lnaGs; omeAs; lnaT |

Key:
lna = Locked Nucleic Acid (LNA); lnamC = LNA 5′ methyl cytosine; ome = 2′-O-methyl; moe = 2′-O-(2-methoxyethyl); moemC = 2′-O-(2-methoxyethyl) 5′ methyl cytosine; s = phosphorothioate linkage; o = phosphodiester linkage.

RNA Sequencing.

RNA from GM09677 fibroblasts that were transfected with Oligo 63, SUZ12 gapmer ASO, and lipid controls. were sequenced (300 bp paired-end) on the NextSeq500 using Illumina TruSeq stranded total RNA-seq library preparation kits.

Northern Blots.

RNA preparation: Total RNA from human fetal brain and lung tissue was obtained from ClonTech and treated with RiboMinus (Life Technologies). 500 ng of rRNA-depleted RNA was fractionated on a 1% agarose gel in 1×MOPS buffer. RNA was capillary transferred to BrightStar Plus nylon membrane (Ambion) overnight in 20×SSC buffer, then crosslinked by UV exposure. For mouse Northern blots, RNA was isolated from 5025 WT brain tissue and WT brain tissue, and treated with RiboMinus as above. Approximately 750 ng RNA was loaded per lane.

Probe Preparation.

DNA templates containing a T7 promoter for in vitro synthesis of radiolabeled RNA probes were generated by PCR from a human fetal brain cDNA library or mouse brain cDNA library with primer pairs listed in the Table 2 or SMN-FL (Hua et al., 2010).

TABLE 2
Probes and Primers

| Probe or Primer Name | Sequence | SEQ ID NO |
|---|---|---|
| PRC2 binding region T7 | TAATACGACTCACTATAGTCCCCTAAACAAAGAC | 30 |
| Forward | GAGGTC | |
| PRC2 binding region Reverse | ATACTGTGTATTGGGATGGGGT | 31 |
| non binding region T7 | TAATACGACTCACTATAGAAAATCAGCCCCCTGA | 32 |
| Forward | GACCAA | |
| non binding region Reverse | TTTTCGAGATGGAGTCTTGCTCTG | 33 |
| RepA I-IV T7 Forward | TAATACGACTCACTATAGATTGTTTATATATTCTT | 34 |
| | GCCCATCGGGG | |
| RepA I-IV Reverse | CACAAAACCATATTTCCATCCACCAAGC | 35 |
| MBP T7 Forward | TAATACGACTCACTATAGATGAAAATAAAAACAG | 36 |
| | GTGCAC | |
| MBP Reverse | CAGATCTTTGTTATAAATCAGCGATAACG | 37 |
| RT-PCR primer or probe | | |
| name | Sequence | |
| SMN-AS1 F (set 1) | GCAGTGCTCTTGTAGTCCCA | 38 |
| SMN-AS1 R (set 1) | CCTCCTTATGGCATAGACACC | 39 |
| SMN-AS1 probe (set 1) | CTTCTGCCAGGAAAGAAGGCAACC | 40 |
| SMN2-FL forward primer | GCTGATGCTTTGGGAAGTATGTTA | 41 |
| SMN2-FL reverse primer | CACCTTCCTTCTTTTTGATTTTGTC | 42 |
| SMN2-FL probe | TACATGAGTGGCTATCATACT | 43 |
| Δ7 SMN2 forward primer | TGGACCACCAATAATTCCCC | 44 |
| Δ7 SMN2 reverse primer | ATGCCAGCATTTCCATATAATAGCC | 45 |
| Δ7 SMN2 probe | TCCAGATTCTCTTGATGATG | 46 |
| ChIP Primer name | Sequence | |
| Exon 2B Forward | CATTTGTGAAACTTCGGGTAAACCA | 47 |
| Exon 2B Reverse | GTAAGGAAGCTGCAGTATTCTTCTTTTG | 48 |
| Exon 2B Probe | CACACCTAAAAGAAAACC | 49 |
| Exon 3/4 Forward | TTTACCCAGCTACCATTGCTTCAA | 50 |
| Exon 3/4 Reverse | CGGACAGATTTTGCTCCTCTCTATT | 51 |
| Exon 3/4 Probe | ACCTGTGTTGTGGTTTAC | 52 |
| Exon 5 Forward | CCTTCTGGACCACCAGTAAGTAAAAA | 53 |
| Exon 5 Reverse | GGGATGTTCTACAATGACATTTTACAATCC | 54 |
| Exon 5 Probe | TTGCTTTCACATACAATTTG | 55 |
| Exon 6 Forward | ATCACTCAGCATCTTTTCCTGACAA | 56 |
| Exon 6 Reverse | GCCTCAGACAGTTGTATTTTTTTATTTTTATTTTTT | 57 |
| | AGTAATATA | |
| Exon 6 Probe | ATGTGACTTTGTTTTGTAAATTTA | 58 |
| Exon 7 Forward | AAAATGTCTTGTGAAACAAAATGCTTTTTA | 59 |
| Exon 7 Reverse | CCTTCTTTTTGATTTTGTCTGAAACCTGTA | 60 |
| Exon 7 Probe | AAAATAAAGGAAGTTAAAAAAAATAG | 61 |
| Exon 8 Forward | CGGTGGTGAGGCAGTTGA | 62 |
| Exon 8 Reverse | CCCTTCTCACAGCTCATAAAATTACCAATAAT | 63 |
| Exon 8 Probe | AATCCACATTCAAATTTTC | 64 |
| Down Forward | TCCCATTTTGTAGGTTGCCTGTT | 65 |
| Down Reverse | ACTAAAGAGCTTCTGCACAGCAAA | 66 |
| Down Probe | CACTCTGATGGTAGTTTCT | 67 |
| Down 2 Forward | CAGCCTCCTGAGTAGCTAGGATTA | 68 |
| Down 2 Reverse | GGTGAAACCCCGTCTCTACTAAAAAA | 69 |
| Down2 Probe | CAGGCACACGCCACCAT | 70 |
| HOXC13prom Forward | GAGACTTCAGCAGTCACAGTGAT | 71 |
| HOXC13prom Reverse | GGAGGAGAGCGCTGTAACT | 72 |
| HOXC13prom Probe | TCCGGTGCACATCCTA | 73 |

Cell Culture for RNA-FISH.

GM09677 Human Eye Lens Fibroblast (Coriell) adherent cells were grown in Eagle's Minimum Essential Medium (EMEM) (ATCC) in a humidified 37° C. incubator at 5% CO₂. F-12K and EMEM media were supplemented with 10% FBS (Fisher Product number SH30071.03), 5 mL of Pen/Strep (Life technologies). F-12 was further supplemented with Normocin (InvivoGen). Cells were grown on 12 mm microscope circular cover glass No. 1 (Fisher #12-545-80) in 24 well flat bottom cell culture plates (E&K). Stellaris® RNA FISH

Probe sets were designed against genomic regions listed in Table 2. They were labeled with Quasar 570® (SMN1/2 exons), Quasar 670 (SMN1/2 introns), and Cal Fluor® Red 610 (SMN1/2-AS1). Stellaris RNA fluorescence in situ hybridization (FISH) was performed as described in the Alternative Protocol for Adherent Cells (UI-207267 Rev. 1.0) with the following modifications: 12 mm diameter coverslips were used. 25 μL hybridization solution was used with a final concentration of each probe set of 250 nM. The wash buffer volumes were halved. The FITC, Cy3, Cy3.5, and Cy5.5 channels were used to capture the signals from each probe set and the FITC channel was used to identify cellular autofluorescence. The filter sets from Chroma were: 49001-ET-FITC, SP102v1-Cy3, SP103v2-Cy3.5, and 41023-Cy5.5. The exposure times were 1 sec for FITC, Quasar 570, and Cal Fluor Red 610, and 2 sec for Quasar 670.

Oligonucleotide Transfection for FISH.

SMA fibroblasts were transfected at 70% confluence by using oligonucleotides complexed with Invitrogen Lipofectamine 3000 (Pub Part #100022234, Pub # MAN0009872, Rev. B.0), and fixed after two days. 2 ng DNA and 4 μL P3000 reagent was used per 50 μL of DNA master mix was. 0.375 μL Lipofectamine 3000 reagent was used per 25 μL of Opti-MEM.

RT-qPCR.

Total RNA from 20 human tissues (Clontech) were used for cDNA synthesis using High Capacity cDNA Reverse Transcription Kit (Applied Biosystems). RT-qPCR SMN-AS1 levels data were normalized to levels from adrenal gland. GM09677 fibroblasts were plated a 24-well tissue culture plate at 4×10⁴ cells/well in MEM containing 10% FBS and 1× non-essential amino acids. Fibroblasts were treated with ASOs the following day. After 2 days cells were lysed and mRNA was purified using E-Z 96 Total RNA Kit (Omega Bio-Tek). SMA iPS-derived motor neurons were lysed with TRIzol for RNA isolation according to the manufacturer's protocol. RNA from mouse cortical neurons was extracted using the RNeasy kit (Qiagen) according to the manufacturers protocol. All cDNAs were synthesized using High Capacity cDNA Reverse Transcription Kit (Applied Biosystems). SMN FL, SMN 47, and SMN Exon 1-2, and GUSB mRNA expression was quantified by predesigned TaqMan real-time PCR assays. A list of custom-designed real-time PCR assays is listed in Table 2.

Oligonucleotide Transfection for ChIP.

SMA fibroblasts were transfected at 70% confluence by using oligonucleotides complexed with Lipofectamine 2000 (Invitrogen) following the protocol suggested by the manufacturer in the 96-well and 24-well format. For ChIP, cells were transfected in 15 cm plate and were transfected at 30 nM with Lipofectamine 2000 at a final volume of 20 mL. Cells were harvested 3 days post transfection.

RNA Immunoprecipitation.

RIP was performed using the Magna RIP RNA-Binding Protein Immunoprecipitation Kit (EMD Millipore) using a ChIP-grade anti-SUZ12 (Abcam), and anti-EZH2 (Abcam). RNA was extracted with Trizol (Life Technologies) and transcribed to cDNA using the High Capacity cDNA Reverse Transcription Kit (Applied Biosystems). qPCR was performed on a StepOnePlus Real Time PCR System (Applied Biosystems) using Taqman Fast Advanced Mastermix (Applied Biosystems).

Electrophoretic Mobility Shift Assay.

DNA templates for EMSA probes containing T7 promoter sequences were generated by PCR using Phusion High Fidelity DNA Polymerase (NEB) and the specific primer sequences are listed in the Table 2. EMSAs were performed as described previously (Cifuentes-Rojas et al., 2014). Briefly, RNA probes were transcribed using the AmpliScribe T7 Flash Transcription Kit (Epicentre) and PAGE purified from 6% TBE urea gel. RNA probes were then dephosphorylated by calf intestinal alkaline phosphatase (NEB), purified by phenol-chloroform extraction, 5′ end-labeled with T4 Polynucleotide Kinase (NEB) and [γ-³²P]ATP (Perkin-Elmer), and purified with Illustra MicroSpin G-50 columns (GE Life Sciences). RNA probes were folded in 10 mM Tris pH 8.0, 1 mM EDTA, 300 mM NaCl by heating to 95° C., followed by incubations at 37° C. and at room temperature for 10 min each. MgCl2 and Hepes pH 7.5 were then added to 10 mM each and probes were put on ice. 1 μl of 2,000 cpm/ml (2 nM final concentration) folded RNA was mixed with PRC2 (EZH2/SUZ12/EED; BPS Bioscience) at the indicated concentration and 50 ng/ml yeast tRNA (Ambion) in 20 μl final concentration of binding buffer (50 mM Tris-HCl pH 8.0, 100 mM NaCl, 5 mM MgCl2, 10 mg/ml BSA, 0.05% NP40, 1 mM DTT, 20 U RNaseOUT [Invitrogen], and 5% glycerol). Binding reactions were incubated for 20 min at 30° C. and applied on a 0.4% hyper-strength agarose (Sigma) gel in THEM buffer (66 mM HEPES, 34 mM Tris, 0.1 mM disodium EDTA, and 10 mM MgCl₂). Gels were run for 1 hr at 130 V with buffer recirculation at 4° C., dried and exposed to a phosphorimager screen. Screens were scanned in a Storm 860 phosphorimager (Molecular Dynamics), data were quantified by Quantity One and normalized as described (Wong and Lohman, 1993). K_{D}s were calculated with Graphpad Prism by fitting the data to a one-site specific binding model.

Western Blot.

Five days post-transfection cells were lysed using the extraction buffer from the SMN ELISA kit (Enzo) with Protease inhibitor cocktail tablets (Roche). The total protein content was determined with the total BCA assay (Promega). Samples and Hi Mark prestained ladder (Invitrogen) were then run on the Bis-tris gel and the protein was transferred to nitrocellulose membrane. Non-specific binding was blocked using blocking buffer from Licor overnight at 4° C. SMN antibody (BD Catalog #610646) and Alpha tubulin antibody (abcam catalog # ab125267) and secondary anti-mouse and anti-rabbit-800 (Licor) were used and the blot was read using a LICOR-odyssey. Band intensities for SMN-FL protein and α-tubulin were quantified using Image Studio software.

ELISA Protocol.

GM09677 fibroblasts were plated a 24-well tissue culture plate at 4×10⁴ cells/well in MEM containing 10% FBS and 1× non-essential amino acids. Fibroblasts were treated with oligonucleotides the following day. After 5 days, cells were lysed and protein was quantified with the SMN ELISA Kit (Enzo Life Sciences, Inc.) and normalized to total protein content as determined by Micro BCA Protein Assay Kit (Thermo Scientific). For the human-specific ELISA used with the cortical neurons, a similar protocol was used. Briefly, cells were washed in cold PBS and lysed in RIPA buffer supplemented with protease inhibitor cOmplete Tablets, mini EDTA-free EASYpack (Roche). Lysates were quantified by BCA and approximately 20-30 μg were used. A mouse monoclonal anti-SMN antibody was captured on high binding plates (Pierce) at 1 μg/mL; after blocking with BSA in PBS-0.05% Tween-20, lysates were incubated for 2 hours at RT; a rabbit polyclonal human SMN-specific antibody at 1 μg/mL was used for detection, followed by HRP-goat anti-rabbit (Invitrogen). The signal was measured with SuperSignal ELISA PICO chemiluminescent substrate (Thermo). Total GAPDH in the lysates was also quantified by ELISA (R&D Systems); SMN protein concentration was normalized to total GAPDH content.

Cortical Neuron Isolation.

Brains were isolated from E14 SMNA7 embryos and the cortex was dissected with the MACS neuronal tissue dissociation kit (Miltenyi Biotec). The collected cortical neurons were plated at 0.5×10⁶ cells per well in Neurobasal media (ThermoFisher), B-27 supplement (Thermofisher), and GlutaMax (ThermoFisher) in a 24-well plate coated with poly-D-lysine (Fisher). Cells were incubated at 37° C., 5% CO₂ for 4 days, allowing the cells to mature and networks to form before unassisted delivery of Oligo 63. After 14 days, the cells were harvested for RNA isolation.

iPS Cell Culturing and Motor Neuron Differentiation Protocol.

SMA patient and control subject dermal fibroblasts or lymphoblastoid cell lines (LCLs) were obtained from the Coriell Institute for Medical Research. The iPSCs were grown to near confluence under normal maintenance conditions before the start of the differentiation as per protocols described previously (PMID: 25298370). Briefly, IPSCs were gently lifted by Accutase treatment for 5 min at 37° C. 1.5-2.5×10⁴ cells were subsequently placed in each well of a 384 well plate in defined neural differentiation medium with dual-SMAD inhibition (PMID:19252484). After 2 days, neural aggregates were transferred to low adherence flasks. Subsequently, neural aggregates were plated onto laminin-coated 6-well plates to induce rosette formation in media supplemented with 0.1 μM retinoic acid and 1 μM puromorphine along with 20 ng/ml BDNF, 200 ng/ml ascorbic acid, 20 ng/ml GDNF and 1 mM dbcAMP. Neural rosettes were isolated and the purified rosettes were subsequently supplemented with 100 ng/mL of EGF and FGF. These neural aggregates, termed iPSC-derived motor neuron precursor spheres (iMPS), were expanded over a 5 week period. For terminal differentiation, iMPS were disassociated with accutase and then plated onto laminin-coated plates over a 21 day period prior to harvest using the MN maturation media consisting of Neurobasal supplemented with 1% N₂, ascorbic acid (200 ng/ml), dibutyryl cyclic adenosine monophosphate (1 μM), BDNF (10 ng/ml), and GDNF (10 ng/ml). Oligo 63 treatments were carried out during this terminal differentiation period. Antibodies used for immunocytochemistry were as follows: SSEA4 and SOX2 (Millipore); TRA-1-60, TRA-1-81, OCT4, NANOG (Stemgent); TuJ1 (β3-tubulin) and Map2 a/b (Sigma); ISLET1 (R&D Systems); and SMI32 (Covance).

Chromatin Immunoprecipitation.

Cells were crosslinked with 1% formaldehyde for 10 minutes at room temperature and then quenched with glycine. Chromatin was prepared and sonicated (Covaris S200) to a size range of 300-500 bp. Antibodies for H3, H3K27me3, H3K36me3, EZH2, and RNA Polymerase II Serine 2 (Abcam) and H3K4me3 (Millipore) were coupled to Protein G magnetic beads (NEB), washed, and then resuspended in IP blocking buffer. Chromatin lysates were added to the beads and immunoprecipitated overnight at 4° C. Antibodies against H3, H3K36me3, RNA Polymerase II phosphoserine 2, H3K27me3, and EZH2 were obtained from Abcam and the H3K4m3 antibody was obtained from Millipore. 10 ug of antibody was used per IP. IPs were washed, RNase A-treated (Roche), Proteinase K-treated (Roche), and then the crosslinks were reversed by incubation overnight at 65° C. DNA was purified, precipitated, and resuspended in nuclease-free water. Custom Taqman probe sets were used to determine DNA enrichment. Probes were designed using the custom design tool on the Life Technologies website. Primer sequences are listed in Table 2.

Bioinformatics Methods.

Mock, Oligo 63, and the SUZ12-KD gapmer treated GM09677 SMA fibroblasts were sequenced (151 bp paired-end) on an Illumina NextSeq 500 machine using the Illumina TruSeq polyA stranded RNAseq library preparation kits. For each of the treatments, there were two time points with each time point having two replicates for a total number of 4 samples per condition. FastQC (Andrews S. (2010). FastQC: a quality control tool for high throughput sequence data. Available online at: bioinformatics.babraham.ac.uk/projects/fastqc) was used to examine fastq quality metrics. Adapter and low quality sequences were trimmed from the reads using Trimmomatic (version 0.35) [PubMed ID (PMID): 24695404] with the following modules and settings: Crop to paired end length of 150 bp; IlluminaClip allowing for 2 seed mismatches, paired end seed score of 30, single end seed score of 10, minimum adapter length of 2, and while keeping both reads; SlidingWindow with a window size of 10 bp and sliding window minimum average phred score of 15; and finally reads were discarded if their length went below 36 base pairs. Next, rRNA reads were removed after aligning against rRNA sequences with bowtie2 v. 2.1.0 [PMID: 22388286].

The rRNA-depleted RNAseq fastq files were aligned with the STAR aligner (version 2.5.1a) [PMID: 23104886] to a modified version of hg38 Homo sapiens reference genome with a chromosomal segment duplication containing SMN2 (chr5:69,924,952-70,129,737) masked in order to align all SMN mapping reads to SMN1 and avoid multimapping. HTseq-count version 0.6.1 [PMID: 25260700] counted reads that overlapped gene features in the Gencode v24 gene annotation [PMID: 22955987 and PMID: 16925838].

Read counts were imported into R version 3.2.3 (R Core Team (2015). R: A language and environment for statistical computing. R Foundation for Statistical Computing, Vienna, Austria. R-project.org/) and were analyzed using Bioconductor [PMID: 25633503]. Lowly expressed genes across the samples were filtered using a mixture model from the SCAN.UPC R package version 2.12.1 [PMID: 24128763]. The remaining feature counts were scaled with TMM normalization [PMID: 20196867] and voom transformed [PMID: 24485249]. Limma version 3.26.7 [PMID: 25605792] was used to fit a linear model blocking time and looking at each oligo treatment vs. mock to identify significant changes in differential expression at an FDR (Benjamini Y., Hochberg Y. Controlling the false discovery rate: a practical and powerful approach to multiple testing. J Roy Statist Soc Ser B (Methodological) 1995; 57:289-300) corrected p value<0.05 with a FC>1.5. Scatter plots were generated using the ggplot2 R package 9H. Wickham. ggplot2: elegant graphics for data analysis. Springer New York, 2009.).

Pathway gene sets were obtained from the canonical pathway (C2) collection in the Molecular Signatures Database (MSigDB v5.0) [PMID: 16199517]. Significant pathways were identified using the competitive gene set testing method Camera with inter gene correlation set to 0.01 and with the same design matrix that was used in the differential expression analysis [PMID: 22638577]. A pathway was considered significant if it met a q value threshold <0.10. Barcode plots of the specific pathways were created using the barcodeplot function. Lastly, overrepresentation of differentially expressed genes or pathways between the different oligonucleotide treatments was evaluated with the hypergeometric test.

### REFERENCES

Agger, K., Cloos, P.A., Christensen, J., Pasini, D., Rose, S., Rappsilber, J., Issaeva, I., Canaani, E., Salcini, A. E., and Helin, K. (2007). UTX and JMJD3 are histone H3K27 demethylases involved in HOX gene regulation and development. Nature 449, 731-734.
Basu, A., Dasari, V., Mishra, R. K., and Khosla, S. (2014). The CpG island encompassing the promoter and first exon of human DNMT3L gene is a PcG/TrX response element (PRE). PLoS One 9, e93561.
Battaglia, G., Princivalle, A., Forti, F., Lizier, C., and Zeviani, M. (1997). Expression of the SMN gene, the spinal muscular atrophy determining gene, in the mammalian central nervous system. Hum Mol Genet 6, 1961-1971.
Boda, B., Mas, C., Giudicelli, C., Nepote, V., Guimiot, F., Levacher, B., Zvara, A., Santha, M., LeGall, I., and Simonneau, M. (2004). Survival motor neuron SMN1 and SMN2 gene promoters: identical sequences and differential expression in neurons and non-neuronal cells. Eur J Hum Genet 12, 729-737.
Burghes, A. H., and Beattie, C. E. (2009). Spinal muscular atrophy: why do low levels of survival motor neuron protein make motor neurons sick? Nat Rev Neurosci 10, 597-609.
Cherry, J. J., and Androphy, E. J. (2012). Therapeutic strategies for the treatment of spinal muscular atrophy. Future Med Chem 4, 1733-1750.
Chiriboga, C. A., Swoboda, K. J., Darras, B. T., Iannaccone, S. T., Montes, J., De Vivo, D. C., Norris, D. A., Bennett, C. F., and Bishop, K. M. (2016). Results from a phase 1 study of nusinersen (ISIS-SMNRx) in children with spinal muscular atrophy. Neurology 86, 890-897.
Cifuentes-Rojas, C., Hernandez, A. J., Sarma, K., and Lee, J. T. (2014). Regulatory interactions between RNA and polycomb repressive complex 2. Mol Cell 55, 171-185.
Davidovich, C., and Cech, T. R. (2015). The recruitment of chromatin modifiers by long noncoding RNAs: lessons from PRC2. Rna 21, 2007-2022.
Davidovich, C., Wang, X., Cifuentes-Rojas, C., Goodrich, K. J., Gooding, A. R., Lee, J. T., and Cech, T. R. (2015). Toward a consensus on the binding specificity and promiscuity of PRC2 for RNA. Mol Cell 57, 552-558.
Di Croce, L., and Helin, K. (2013). Transcriptional regulation by Polycomb group proteins. Nat Struct Mol Biol 20, 1147-1155.
Feldkotter, M., Schwarzer, V., Wirth, R., Wienker, T. F., and Wirth, B. (2002). Quantitative analyses of SMN1 and SMN2 based on real-time lightCycler PCR: fast and highly reliable carrier testing and prediction of severity of spinal muscular atrophy. Am J Hum Genet 70, 358-368.
Hua, Y., Sahashi, K., Hung, G., Rigo, F., Passini, M. A., Bennett, C. F., and Krainer, A. R. (2010). Antisense correction of SMN2 splicing in the CNS rescues necrosis in a type III SMA mouse model. Genes Dev 24, 1634-1644.
Kadoch, C., Copeland, R. A., and Keilhack, H. (2016). PRC2 and SWI/SNF Chromatin Remodeling Complexes in Health and Disease. Biochemistry 55, 1600-1614.
Kauppinen, S., Vester, B., and Wengel, J. (2005). Locked nucleic acid (LNA): High affinity targeting of RNA for diagnostics and therapeutics. Drug Discov Today Technol 2, 287-290.
Kotake, Y., Nakagawa, T., Kitagawa, K., Suzuki, S., Liu, N., Kitagawa, M., and Xiong, Y. (2011). Long non-coding RNA ANRIL is required for the PRC2 recruitment to and silencing of p15(INK4B) tumor suppressor gene. Oncogene 30, 1956-1962.
Le, T. T., Pham, L. T., Butchbach, M. E., Zhang, H. L., Monani, U. R., Coovert, D. D., Gavrilina, T. O., Xing, L., Bassell, G. J., and Burghes, A. H. (2005). SMNDelta7, the major product of the centromeric survival motor neuron (SMN2) gene, extends survival in mice with spinal muscular atrophy and associates with full-length SMN. Hum Mol Genet 14, 845-857.
Lefebvre, S., Burlet, P., Liu, Q., Bertrandy, S., Clermont, O., Munnich, A., Dreyfuss, G., and Melki, J. (1997). Correlation between severity and SMN protein level in spinal muscular atrophy. Nat Genet 16, 265-269.
Luco, R. F., Pan, Q., Tominaga, K., Blencowe, B. J., Pereira-Smith, O. M., and Misteli, T. (2010). Regulation of alternative splicing by histone modifications. Science 327, 996-1000.
Margueron, R., Li, G., Sarma, K., Blais, A., Zavadil, J., Woodcock, C. L., Dynlacht, B. D., and Reinberg, D. (2008). Ezh1 and Ezh2 maintain repressive chromatin through different mechanisms. Mol Cell 32, 503-518.
Monani, U. R. (2005). Spinal muscular atrophy: a deficiency in a ubiquitous protein; a motor neuron-specific disease. Neuron 48, 885-896.
Palacino, J., Swalley, S. E., Song, C., Cheung, A. K., Shu, L., Zhang, X., Van Hoosear, M., Shin, Y., Chin, D. N., Keller, C. G., et al. (2015). SMN2 splice modulators enhance U1-pre-mRNA association and rescue SMA mice. Nat Chem Biol 11, 511-517.
Rossoll, W., Kroning, A. K., Ohndorf, U. M., Steegborn, C., Jablonka, S., and Sendtner, M. (2002). Specific interaction of Smn, the spinal muscular atrophy determining gene product, with hnRNP-R and gry-rbp/hnRNP-Q: a role for Smn in RNA processing in motor axons? Hum Mol Genet 11, 93-105.
Simon, J. A., and Kingston, R. E. (2013). Occupying chromatin: Polycomb mechanisms for getting to genomic targets, stopping transcriptional traffic, and staying put. Mol Cell 49, 808-824.
Sing, A., Pannell, D., Karaiskakis, A., Sturgeon, K., Djabali, M., Ellis, J., Lipshitz, H. D., and Cordes, S. P. (2009). A vertebrate Polycomb response element governs segmentation of the posterior hindbrain. Cell 138, 885-897.
Vitte, J., Fassier, C., Tiziano, F. D., Dalard, C., Soave, S., Roblot, N., Brahe, C., Saugier-Veber, P., Bonnefont, J. P., and Melki, J. (2007). Refined characterization of the expression and stability of the SMN gene products. Am J Pathol 171, 1269-1280.
Vizan, P., Beringer, M., Ballare, C., and Di Croce, L. (2015). Role of PRC2-associated factors in stem cells and disease. Febs J 282, 1723-1735.
Woo, C. J., Kharchenko, P. V., Daheron, L., Park, P. J., and Kingston, R. E. (2010). A region of the human HOXD cluster that confers polycomb-group responsiveness. Cell 140, 99-110.
Woo, C. J., Kharchenko, P. V., Daheron, L., Park, P. J., and Kingston, R. E. (2013). Variable requirements for DNA-binding proteins at polycomb-dependent repressive regions in human HOX clusters. Mol Cell Biol 33, 3274-3285.
Zhang, E. B., Yin, D. D., Sun, M., Kong, R., Liu, X. H., You, L. H., Han, L., Xia, R., Wang, K. M., Yang, J. S., et al. (2014). P53-regulated long non-coding RNA TUG1 affects cell proliferation in human non-small cell lung cancer, partly through epigenetically regulating HOXB7 expression. Cell Death Dis 5, e1243.
Zhao, J., Ohsumi, T. K., Kung, J. T., Ogawa, Y., Grau, D. J., Sarma, K., Song, J. J., Kingston, R. E., Borowsky, M., and Lee, J. T. (2010). Genome-wide identification of polycomb-associated RNAs by RIP-seq. Mol Cell 40, 939-953.

The foregoing written specification is considered to be sufficient to enable one skilled in the art to practice the disclosure. The present disclosure is not to be limited in scope by examples provided, since the examples are intended as a single illustration of one aspect of the disclosure and other functionally equivalent embodiments are within the scope of the disclosure. Various modifications of the disclosure in addition to those shown and described herein will become apparent to those skilled in the art from the foregoing description and fall within the scope of the appended claims. The advantages and objects of the disclosure are not necessarily encompassed by each embodiment of the disclosure.

What is claimed is:

1. A compound for increasing expression of SMN protein in a human cell, the compound comprising:
a first oligonucleotide comprising at least 8 contiguous nucleotides complementary with the sequence set forth as: ATCTGTTCCACTATG (SEQ ID NO: 1); and
a second oligonucleotide that is complementary with a splice control sequence of SMN2 pre-messenger RNA and that promotes inclusion of exon 7 of the SMN2 pre-messenger RNA, wherein the first and second oligonucleotides are covalently linked.

2. The compound of claim 1, wherein the first and second oligonucleotides are covalently linked via an oligonucleotide linker.

3. The compound of claim 1 or 2, wherein the oligonucleotide linker comprises a sequence set forth as Wₙ, wherein W is a nucleotide selected from A, T, and U, and n is a integer selected from 2, 3 and 4, representing the number of instances of W.

4. The compound of claim 3, wherein each instance of W is A.

5. The compound of claim 4, wherein n is 2 or 3.

6. The compound of claim 4 or 5, wherein the oligonucleotide linker comprises phosphodiester bonds between each instance of W.

7. The compound of any one of claims 1 to 6, wherein the first oligonucleotide has a length in a range of 8 to 14 nucleotides.

8. The compound of any one of claims 1 to 7, wherein the first oligonucleotide has a length in a range of 8 to 10 nucleotides.

9. The compound of any one of claims 1 to 8, wherein the first oligonucleotide comprises at least 8 contiguous nucleotides of the sequence set forth as: AGUGGAACA.

10. The compound of any one of claims 1 to 9, wherein the second oligonucleotide comprises a region of complementarity complementary with at least 8 contiguous nucleotides of the sequence set forth as: GUAAGUCUGCCAGCAUUAUGAAAG (SEQ ID NO: 2).

11. The compound of any one of claims 1 to 10, wherein the region of complementarity is complementary with at least 8 contiguous nucleotides of the sequence set forth as: CUGCCAGCAUUAUGAAAG (SEQ ID NO: 3).

12. The compound of any one of claims 1 to 11, wherein the region of complementarity is complementary with at least 8 contiguous nucleotides of the sequence set forth as: CCAGCAUUAUGAAAG (SEQ ID NO: 4).

13. The compound of any one of claims 1 to 12, wherein the second oligonucleotide has a sequence set forth as TCACTTTCATAATGC (SEQ ID NO: 17).

14. The compound of any one of claims 1 to 12, wherein the second oligonucleotide has a sequence set forth as ACTTTCATAATGCTGG (SEQ ID NO: 20).

15. The compound of any one of claims 1 to 11, wherein the region of complementarity is complementary with the sequence set forth as: CUGCCAGC.

16. The compound of any one of claims 1 to 13, wherein the first oligonucleotide has a sequence set forth as CATAGTGGAACAGAT (SEQ ID NO: 14) and the second oligonucleotide has a sequence set forth as GCUGGCAG or GCTGGCAG, wherein the first oligonucleotide and the second oligonucleotide linker are covalently linked by an oligonucleotide linker.

17. The compound of claim 16, wherein the oligonucleotide linker has a sequence of AA or AAA.

18. The compound of any one of claims 1 to 17, wherein each nucleotide of the first oligonucleotide is a 2′-modified nucleotide.

19. The compound of any one of claims 1 to 18, wherein each nucleotide of the second oligonucleotide is a 2′-modified nucleotide.

20. The compound of claim 18 or 19, wherein at least one 2′-modified nucleotide is a bridged nucleotide comprising a 2′-4′ methylene bridge.

21. The compound of any one of claims 1 to 20, wherein at least 60% of the nucleotides of the first oligonucleotide are bridged nucleotides.

22. The compound of any one of claims 1 to 21, wherein at least 60% of the nucleotides of the second oligonucleotide are bridged nucleotides.

23. The compound of any one of claims 20 to 22, wherein each bridged nucleotide comprises a 2′-4′ methylene bridge.

24. The compound of any one of claims 1 to 23, wherein the first oligonucleotide comprises at least one phosphorothioate internucleotide linkage.

25. The compound of any one of claims 1 to 24, wherein the second oligonucleotide comprises at least one phosphorothioate internucleotide linkage.

26. A composition for increasing expression of SMN protein, the composition comprising:
i) a first oligonucleotide having a nucleotide sequence consisting of 8 to 14 contiguous nucleotides complementary with the nucleotide sequence set forth as: ATCTGTTCCACTATG (SEQ ID NO: 1); and
ii) an SMN splice correcting agent that promotes inclusion of exon 7 of the SMN2 pre-messenger RNA.

27. The composition of claim 26, wherein the SMN splice correcting agent is a small molecule or an oligonucleotide.

28. The composition of claim 27, wherein the SMN splice correcting agent is a second oligonucleotide that is complementary with a splice control sequence of SMN2 pre-messenger RNA and that promotes inclusion of exon 7 of the SMN2 pre-messenger RNA.

29. The composition of claim 26, wherein the first oligonucleotide has a length in a range of 8 to 10 nucleotides.

30. The composition of any one of claims 26 to 29, wherein the first oligonucleotide comprises at least 8 contiguous nucleotides of the sequence set forth as: AGUGGAACA.

31. The composition of any one of claims 28 to 30, wherein the second oligonucleotide comprises a region of complementarity complementary with at least 8 contiguous nucleotides of the sequence set forth as: GUAAGUCUGCCAGCAUUAUGAAAG (SEQ ID NO: 2).

32. The composition of any one of claims 28 to 31, wherein the region of complementarity is complementary with at least 8 contiguous nucleotides of the sequence set forth as: CUGCCAGCAUUAUGAAAG (SEQ ID NO: 3).

33. The composition of any one of claims 28 to 32, wherein the region of complementarity is complementary with at least 8 contiguous nucleotides of the sequence set forth as: CCAGCAUUAUGAAAG (SEQ ID NO: 4).

34. The composition of any one of claims 28 to 33, wherein the second oligonucleotide has a sequence set forth as TCACTTTCATAATGC (SEQ ID NO: 17).

35. The composition of any one of claims 28 to 33, wherein the second oligonucleotide has a sequence set forth as ACTTTCATAATGCTGG (SEQ ID NO: 20).

36. The composition of any one of claims 28 to 33, wherein the region of complementarity of the second oligonucleotide is complementary with the sequence set forth as: CUGCCAGC.

37. The composition of any one of claims 26 to 36, wherein each nucleotide of the first oligonucleotide is a 2′-modified nucleotide.

38. The composition of any one of claims 26 to 37, wherein each nucleotide of the second oligonucleotide is a 2′-modified nucleotide.

39. The composition of claim 37 or 38, wherein at least one 2′-modified nucleotide is a bridged nucleotide comprising a 2′-4′ methylene bridge.

40. The composition of any one of claims 26 to 39, wherein at least 60% of the nucleotides of the first oligonucleotide are bridged nucleotides.

41. The composition of any one of claims 28 to 40, wherein at least 60% of the nucleotides of the second oligonucleotide are bridged nucleotides.

42. The composition of claim 40 or 41, wherein each bridged nucleotide comprises a 2′-4′ methylene bridge.

43. The composition of any one of claims 26 to 42, wherein the first oligonucleotide comprises at least one phosphorothioate internucleotide linkage.

44. The composition of any one of claims 28 to 43, wherein the second oligonucleotide comprises at least one phosphorothioate internucleotide linkage.

45. A method of increasing expression of SMN protein in a cell, the method comprising delivering to the cell a compound or composition of any one of claims 1 to 44 in an amount effective for increasing expression of SMN protein in the cell.

46. A method of treating expression of SMN protein in a cell, the method comprising delivering to the cell an oligonucleotide of any one of claims 1 to 44 in an amount effective for increasing expression of SMN protein in the cell.

47. A method of treating spinal muscular atrophy (SMA) in a subject, the method comprising administering to the subject a composition comprising:
i) an oligonucleotide complementary with a PRC2-associated region of SMN; and
ii) an SMN splice correcting agent.

48. The method of claim 47, wherein the oligonucleotide has a nucleotide sequence consisting of 8 to 14 contiguous nucleotides complementary with the PRC2-associated region SMN.

49. The method of claim 47, wherein the oligonucleotide has a nucleotide sequence consisting of 8 to 14 contiguous nucleotides complementary with the nucleotide sequence set forth as: ATCTGTTCCACTATG (SEQ ID NO: 1);

50. The method of claim 48, wherein the SMN splice correcting agent promotes inclusion of exon 7 of the SMN2 pre-messenger RNA.

51. A method of treating spinal muscular atrophy (SMA) in a subject, the method comprising administering to the subject a composition comprising:
i) a first oligonucleotide having a nucleotide sequence consisting of 8 to 14 contiguous nucleotides complementary with the nucleotide sequence set forth as: ATCTGTTCCACTATG (SEQ ID NO: 1); and
ii) an SMN splice correcting agent that promotes inclusion of exon 7 of the SMN2 pre-messenger RNA.

52. The method of claim 51, wherein the SMN splice correcting agent is a small molecule or an oligonucleotide.

53. The method of claim 51, wherein the SMN splice correcting agent is a second oligonucleotide that is complementary with a splice control sequence of SMN2 pre-messenger RNA and that promotes inclusion of exon 7 of the SMN2 pre-messenger RNA.

54. The method of any one of claims 51 to 53, wherein the first oligonucleotide has a length in a range of 8 to 10 nucleotides.

55. The method of any one of claims 51 to 54, wherein the first oligonucleotide and the SMN splice correcting agent is linked via a linker.

56. The method of claim 55, wherein the linker is an oligonucleotide linker.

57. The method of claim 56, wherein the oligonucleotide linker comprises a sequence set forth as Wₙ, wherein W is a nucleotide selected from A, T, and U, and n is a integer selected from 2, 3 and 4, representing the number of instances of W.

58. The method of claim 57, wherein each instance of W is A.

59. The method of claim 57 or 58, wherein n is 2 or 3.

60. The method of any one of claims 51 to 59, wherein the first oligonucleotide and the SMN splice correcting agent are separated.


SEQUENCE LISTING (73 sequences)

SEQ ID NO: 1 - DNA, 15 bp, Artificial Sequence
  Synthetic Polynucleotide
atctgttccactatg

SEQ ID NO: 2 - RNA, 24 nt, Artificial Sequence
  Synthetic Polynucleotide
guaagucugccagcauuaugaaag

SEQ ID NO: 3 - RNA, 18 nt, Artificial Sequence
  Synthetic Polynucleotide
cugccagcauuaugaaag

SEQ ID NO: 4 - RNA, 15 nt, Artificial Sequence
  Synthetic Polynucleotide
ccagcauuaugaaag

SEQ ID NO: 5 - RNA, 14 nt, Artificial Sequence
  Synthetic Polynucleotide
cagcauuaugaaag

SEQ ID NO: 6 - DNA, 15 bp, Artificial Sequence
  Synthetic Polynucleotide
  misc_feature (1)..(7): LNAs
  misc_feature (8)..(8): Modified with 2′O methyl
  misc_feature (9)..(11): LNAs
  misc_feature (12)..(12): Modified with 2′O methyl
  misc_feature (13)..(13): LNA
  misc_feature (14)..(14): Modified with 2′O methyl
  misc_feature (15)..(15): LNA
catagtggaacagat

SEQ ID NO: 7 - DNA, 18 bp, Artificial Sequence
  Synthetic Polynucleotide
tcactttcataatgctgg

SEQ ID NO: 8 - protein, 4 aa, Artificial Sequence
  Synthetic Poly
  misc_feature (1)..(18): Modified by 2′-O-methoxyethyl
ALAL

SEQ ID NO: 9 - protein, 9 aa, Artificial Sequence
  Synthetic Poly
APISFFELG

SEQ ID NO: 10 - protein, 11 aa, Artificial Sequence
  Synthetic Poly
  MOD_RES (1)..(1): Modified with maleimidocaproyl
GRWHTVGLRWE

SEQ ID NO: 11 - protein, 6 aa, Artificial Sequence
  Synthetic Poly
RRALAL

SEQ ID NO: 12 - protein, 11 aa, Artificial Sequence
  Synthetic Poly
GRWPPMGLPWE

SEQ ID NO: 13 - protein, 11 aa, Artificial Sequence
  Synthetic Poly
GRWHPMGAPWE

SEQ ID NO: 14 - DNA, 15 bp, Artificial Sequence
  Synthetic Polynucleotide
catagtggaacagat

SEQ ID NO: 15 - DNA, 15 bp, Artificial Sequence
  Synthetic Polynucleotide
  misc_feature (1)..(7): LNAs
  misc_feature (8)..(8): Modified with 2′O methyl
  misc_feature (9)..(11): LNAs
  misc_feature (12)..(12): Modified with 2′O methyl
  misc_feature (13)..(13): LNA
  misc_feature (14)..(14): Modified with 2′O methyl
  misc_feature (15)..(15): LNA
catagtggaacagat

SEQ ID NO: 16 - DNA, 15 bp, Artificial Sequence
  Synthetic Polynucleotide
  misc_feature (1)..(1): 5-methylcytosine
  misc_feature (2)..(7): LNAs
  misc_feature (8)..(8): Modified with 2′O methyl
  misc_feature (9)..(10): LNAs
  misc_feature (11)..(11): 5-methylcytosine
  misc_feature (12)..(12): Modified with 2′O methyl
  misc_feature (13)..(13): LNA
  misc_feature (14)..(14): Modified with 2′O methyl
  misc_feature (15)..(15): LNA
catagtggaacagat

SEQ ID NO: 17 - DNA, 15 bp, Artificial Sequence
  Synthetic Polynucleotide
tcactttcataatgc

SEQ ID NO: 18 - DNA, 15 bp, Artificial Sequence
  Synthetic Polynucleotide
  misc_feature (1)..(7): LNAs
  misc_feature (8)..(8): Modified with 2′O methyl
  misc_feature (9)..(11): LNAs
  misc_feature (12)..(12): Modified with 2′O methyl
  misc_feature (13)..(13): LNAs
  misc_feature (14)..(14): Modified with 2′O methyl
  misc_feature (15)..(15): LNA
tcactttcataatgc

SEQ ID NO: 19 - DNA, 15 bp, Artificial Sequence
  Synthetic Polynucleotide
  misc_feature (1)..(1): LNA
  misc_feature (2)..(2): 5-methylcytosine
  misc_feature (3)..(3): LNA
  misc_feature (4)..(4): 5-methylcytosine
  misc_feature (5)..(7): LNAs
  misc_feature (8)..(8): Modified with 2′O methyl
  misc_feature (9)..(11): LNAs
  misc_feature (12)..(12): Modified with 2′O methyl
  misc_feature (13)..(13): LNA
  misc_feature (14)..(14): Modified with 2′O methyl
  misc_feature (15)..(15): LNA
tcactttcataatgc

SEQ ID NO: 20 - DNA, 16 bp, Artificial Sequence
  Synthetic Polynucleotide
actttcataatgctgg

SEQ ID NO: 21 - DNA, 16 bp, Artificial Sequence
  Synthetic Polynucleotide
  misc_feature (1)..(8): LNAs
  misc_feature (9)..(9): Modified with 2′O methyl
  misc_feature (10)..(12): LNAs
  misc_feature (13)..(13): Modified with 2′O methyl
  misc_feature (14)..(14): LNA
  misc_feature (15)..(15): Modified with 2′O methyl
  misc_feature (16)..(16): LNA
actttcataatgctgg

SEQ ID NO: 22 - DNA, 16 bp, Artificial Sequence
  Synthetic Polynucleotide
  misc_feature (1)..(1): LNA
  misc_feature (2)..(2): 5-methylcytosine
  misc_feature (3)..(3): LNA
  misc_feature (4)..(4): 5-methylcytosine
  misc_feature (5)..(8): LNAs
  misc_feature (9)..(9): Modified with 2′O methyl
  misc_feature (10)..(12): LNAs
  misc_feature (13)..(13): Modified with 2′O methyl
  misc_feature (14)..(14): LNA
  misc_feature (15)..(15): Modified with 2′O methyl
  misc_feature (16)..(16): LNA
actttcataatgctgg

SEQ ID NO: 23 - DNA, 25 bp, Artificial Sequence
  Synthetic Polynucleotide
  misc_feature (1)..(1): 5-methylcytosine
  misc_feature (2)..(7): LNAs
  misc_feature (8)..(8): Modified with 2′O methyl
  misc_feature (9)..(10): LNAs
  misc_feature (11)..(11): 5-methylcytosine
  misc_feature (12)..(12): Modified with 2′O methyl
  misc_feature (13)..(13): LNA
  misc_feature (14)..(14): Modified with 2′O methyl
  misc_feature (15)..(15): LNA
  misc_feature (16)..(16): a linker between a single stranded oligonucleotide complementary to a PCR2-associated region and a splice correcting oligonucleotide
  misc_feature (16)..(18): a phosphodiester bond between two nucleotides in a linker or a phosphodiester bond linking one end of a linker to a single stranded oligonucleotide complementary to a PRC2- associated region or a splice correcting oligonucleotide
  misc_feature (17)..(17): a linker between a single stranded oligonucleotide complementary to a PCR2-associated region and a splice correcting oligonucleotide
  misc_feature (18)..(25): Modified with 2′O methyl
catagtggaacagataagcuggcag

SEQ ID NO: 24 - DNA, 26 bp, Artificial Sequence
  Synthetic Polynucleotide
  misc_feature (1)..(1): 5-methylcytosine
  misc_feature (2)..(7): LNAs
  misc_feature (8)..(8): Modified with 2′O methyl
  misc_feature (9)..(10): LNAs
  misc_feature (11)..(11): 5-methylcytosine
  misc_feature (12)..(12): Modified with 2′O methyl
  misc_feature (13)..(13): LNA
  misc_feature (14)..(14): Modified with 2′O methyl
  misc_feature (15)..(15): LNA
  misc_feature (16)..(16): a linker between a single stranded oligonucleotide complementary to a PCR2-associated region and a splice correcting oligonucleotide
  misc_feature (16)..(19): a phosphodiester bond between two nucleotides in a linker or a phosphodiester bond linking one end of a linker to a single stranded oligonucleotide complementary to a PRC2- associated region or a splice correcting oligonucleotide
  misc_feature (17)..(17): a linker between a single stranded oligonucleotide complementary to a PCR2-associated region and a splice correcting oligonucleotide
  misc_feature (18)..(26): Modified with 2′O methyl
  misc_feature (18)..(18): a linker between a single stranded oligonucleotide complementary to a PCR2-associated region and a splice correcting oligonucleotide
catagtggaacagataaagcuggcag

SEQ ID NO: 25 - DNA, 26 bp, Artificial Sequence
  Synthetic Polynucleotide
  misc_feature (1)..(1): 5-methylcytosine
  misc_feature (2)..(7): LNAs
  misc_feature (8)..(8): Modified with 2′O methyl
  misc_feature (9)..(10): LNAs
  misc_feature (11)..(11): 5-methylcytosine
  misc_feature (12)..(12): Modified with 2′O methyl
  misc_feature (13)..(13): LNA
  misc_feature (14)..(14): Modified with 2′O methyl
  misc_feature (15)..(15): LNA
  misc_feature (16)..(16): a linker between a single stranded oligonucleotide complementary to a PCR2-associated region and a splice correcting oligonucleotide
  misc_feature (16)..(19): a phosphodiester bond between two nucleotides in a linker or a phosphodiester bond linking one end of a linker to a single stranded oligonucleotide complementary to a PRC2- associated region or a splice correcting oligonucleotide
  misc_feature (17)..(17): a linker between a single stranded oligonucleotide complementary to a PCR2-associated region and a splice correcting oligonucleotide
  misc_feature (18)..(18): a linker between a single stranded oligonucleotide complementary to a PCR2-associated region and a splice correcting oligonucleotide
  misc_feature (20)..(26): LNAs
catagtggaacagataaagctggcag

SEQ ID NO: 26 - DNA, 15 bp, Artificial sequence
  Synthetic Polynucleotide
agaugcagtgcucut

SEQ ID NO: 27 - DNA, 15 bp, Artificial sequence
  Synthetic Polynucleotide
cataguggaacagat

SEQ ID NO: 28 - DNA, 18 bp, Artificial sequence
  Synthetic Polynucleotide
tcactttcataatgctgg

SEQ ID NO: 29 - DNA, 15 bp, Artificial sequence
  Synthetic Polynucleotide
catagtggaacagat

SEQ ID NO: 30 - DNA, 40 bp, Artificial Sequence
  Synthetic Polynucleotide
taatacgactcactatagtcccctaaacaaagacgaggtc

SEQ ID NO: 31 - DNA, 22 bp, Artificial sequence
  Synthetic Polynucleotide
atactgtgtattgggatggggt

SEQ ID NO: 32 - DNA, 40 bp, Artificial sequence
  Synthetic Polynucleotide
taatacgactcactatagaaaatcagccccctgagaccaa

SEQ ID NO: 33 - skipped

SEQ ID NO: 34 - DNA, 46 bp, Artificial sequence
  Synthetic Polynucleotide
taatacgactcactatagattgtttatatattcttgcccatcgggg

SEQ ID NO: 35 - DNA, 28 bp, Artificial sequence
  Synthetic Polynucleotide
cacaaaaccatatttccatccaccaagc

SEQ ID NO: 36 - DNA, 40 bp, Artificial sequence
  Synthetic Polynucleotide
taatacgactcactatagatgaaaataaaaacaggtgcac

SEQ ID NO: 37 - DNA, 29 bp, Artificial sequence
  Synthetic Polynucleotide
cagatctttgttataaatcagcgataacg

SEQ ID NO: 38 - DNA, 20 bp, Artificial sequence
  Synthetic Polynucleotide
gcagtgctcttgtagtccca

SEQ ID NO: 39 - DNA, 21 bp, Artificial sequence
  Synthetic Polynucleotide
cctccttatggcatagacacc

SEQ ID NO: 40 - DNA, 24 bp, Artificial sequence
  Synthetic Polynucleotide
cttctgccaggaaagaaggcaacc

SEQ ID NO: 41 - DNA, 24 bp, Artificial sequence
  Synthetic Polynucleotide
gctgatgctttgggaagtatgtta

SEQ ID NO: 42 - DNA, 25 bp, Artificial sequence
  Synthetic Polynucleotide
caccttccttctttttgattttgtc

SEQ ID NO: 43 - DNA, 21 bp, Artificial sequence
  Synthetic Polynucleotide
tacatgagtggctatcatact

SEQ ID NO: 44 - DNA, 20 bp, Artificial sequence
  Synthetic Polynucleotide
tggaccaccaataattcccc

SEQ ID NO: 45 - DNA, 25 bp, Artificial sequence
  Synthetic Polynucleotide
atgccagcatttccatataatagcc

SEQ ID NO: 46 - DNA, 20 bp, Artificial sequence
  Synthetic Polynucleotide
tccagattctcttgatgatg

SEQ ID NO: 47 - DNA, 25 bp, Artificial sequence
  Synthetic Polynucleotide
catttgtgaaacttcgggtaaacca

SEQ ID NO: 48 - DNA, 28 bp, Artificial sequence
  Synthetic Polynucleotide
gtaaggaagctgcagtattcttcttttg

SEQ ID NO: 49 - DNA, 18 bp, Artificial sequence
  Synthetic Polynucleotide
cacacctaaaagaaaacc

SEQ ID NO: 50 - DNA, 24 bp, Artificial sequence
  Synthetic Polynucleotide
tttacccagctaccattgcttcaa

SEQ ID NO: 51 - DNA, 25 bp, Artificial sequence
  Synthetic Polynucleotide
cggacagattttgctcctctctatt

SEQ ID NO: 52 - DNA, 18 bp, Artificial sequence
  Synthetic Polynucleotide
acctgtgttgtggtttac

SEQ ID NO: 53 - DNA, 26 bp, Artificial sequence
  Synthetic Polynucleotide
ccttctggaccaccagtaagtaaaaa

SEQ ID NO: 54 - DNA, 30 bp, Artificial sequence
  Synthetic Polynucleotide
gggatgttctacaatgacattttacaatcc

SEQ ID NO: 55 - DNA, 20 bp, Artificial sequence
  Synthetic Polynucleotide
ttgctttcacatacaatttg

SEQ ID NO: 56 - DNA, 25 bp, Artificial sequence
  Synthetic Polynucleotide
atcactcagcatcttttcctgacaa

SEQ ID NO: 57 - DNA, 45 bp, Artificial sequence
  Synthetic Polynucleotide
gcctcagacagttgtatttttttatttttattttttagtaatata

SEQ ID NO: 58 - DNA, 24 bp, Artificial sequence
  Synthetic Polynucleotide
atgtgactttgttttgtaaattta

SEQ ID NO: 59 - DNA, 30 bp, Artificial sequence
  Synthetic Polynucleotide
aaaatgtcttgtgaaacaaaatgcttttta

SEQ ID NO: 60 - DNA, 30 bp, Artificial sequence
  Synthetic Polynucleotide
ccttctttttgattttgtctgaaacctgta

SEQ ID NO: 61 - DNA, 26 bp, Artificial sequence
  Synthetic Polynucleotide
aaaataaaggaagttaaaaaaaatag

SEQ ID NO: 62 - DNA, 18 bp, Artificial sequence
  Synthetic Polynucleotide
cggtggtgaggcagttga

SEQ ID NO: 63 - DNA, 32 bp, Artificial sequence
  Synthetic Polynucleotide
cccttctcacagctcataaaattaccaataat

SEQ ID NO: 64 - DNA, 19 bp, Artificial sequence
  Synthetic Polynucleotide
aatccacattcaaattttc

SEQ ID NO: 65 - DNA, 23 bp, Artificial sequence
  Synthetic Polynucleotide
tcccattttgtaggttgcctgtt

SEQ ID NO: 66 - DNA, 24 bp, Artificial sequence
  Synthetic Polynucleotide
actaaagagcttctgcacagcaaa

SEQ ID NO: 67 - DNA, 19 bp, Artificial sequence
  Synthetic Polynucleotide
cactctgatggtagtttct

SEQ ID NO: 68 - DNA, 24 bp, Artificial sequence
  Synthetic Polynucleotide
cagcctcctgagtagctaggatta

SEQ ID NO: 69 - DNA, 26 bp, Artificial sequence
  Synthetic Polynucleotide
ggtgaaaccccgtctctactaaaaaa

SEQ ID NO: 70 - DNA, 17 bp, Artificial sequence
  Synthetic Polynucleotide
caggcacacgccaccat

SEQ ID NO: 71 - DNA, 23 bp, Artificial sequence
  Synthetic Polynucleotide
gagacttcagcagtcacagtgat

SEQ ID NO: 72 - DNA, 19 bp, Artificial sequence
  Synthetic Polynucleotide
ggaggagagcgctgtaact

SEQ ID NO: 73 - DNA, 16 bp, Artificial sequence
  Synthetic Polynucleotide
tccggtgcacatccta